(19) United States
(12) Patent Application Publication
(10) Pub. No.: US 2024/0376229 A1
(43) Pub. Date: Nov. 14, 2024

(54) INTERFERON RECEPTOR ANTAGONISTS AND USES THEREOF

(71) Applicant: Regeneron Pharmaceuticals, Inc., Tarrytown, NY (US)

(72) Inventors: Vidur GARG, White Plains, NY (US); Nicolin BLOCH, Elmsford, NY (US); Erica ULLMAN, Yorktown Heights, NY (US); Eva-Maria WEICK, New York, NY (US); Jiaxi WU, Pleasantville, NY (US); Tong ZHANG, New Rochelle, NY (US); Eric SMITH, New York, NY (US); Aynur HERMANN, New York, NY (US); Chia-Yang LIN, Scarsdale, NY (US)

(73) Assignee: Regeneron Pharmaceuticals, Inc., Tarrytown, NY (US)

(21) Appl. No.: 18/661,554

(22) Filed: May 10, 2024

Related U.S. Application Data
(60) Provisional application No. 63/597,502, filed on Nov. 9, 2023.
(60) Provisional application No. 63/501,840, filed on May 12, 2023.

(51) Int. Cl. C07K 16/46; A61K 35/76; C07K 14/555; C07K 14/715; C07K 16/28

(52) U.S. Cl. CPC C07K 16/46; A61K 35/76; C07K 14/555; C07K 14/7156; C07K 16/2827; C07K 2317/76; C07K 2319/30

(57) ABSTRACT

The present disclosure provides interferon (IFN) receptor antagonists. IFN receptor antagonists disclosed herein comprise an anchoring moiety, an IFN masking moiety, an IFN moiety, and a separator moiety, e.g., a targeting moiety that recognizes an antigen associated with cells expressing Type 1 interferon receptors and anchors the IFN receptor antagonist to such cells. The disclosure further provides pharmaceutical compositions comprising the IFN receptor antagonists, and methods of use of the IFN receptor antagonists in IFN signaling inhibition, including treatment methods. Also disclosed are nucleic acids encoding the IFN receptor antagonists, recombinant cells that express the IFN receptor antagonists, and methods of producing the IFN receptor antagonists.

### 1. CROSS-REFERENCE TO RELATED APPLICATIONS

This application claims the priority benefit of U.S. provisional application No. 63/501,840, filed May 12, 2023, and U.S. provisional application No. 63/597,502, filed Nov. 9, 2023, the contents of each of which are incorporated herein in their entireties by reference thereto.

### 2. SEQUENCE LISTING

The instant application contains a Sequence Listing which has been submitted electronically in XML format and is hereby incorporated by reference in its entirety. Said Sequence Listing, created on May 6, 2024 is named RGN-035US_SL.xml and is 146,865 bytes in size.

### 3. BACKGROUND

Type I interferons (IFNs) are a family of cytokines that serve as immunomodulators for innate and adaptive immune responses. Most cell types constitutively produce low levels of type I IFNs; however, infections and other triggers can stimulate the production of these molecules, which then bind to and signal through IFNAR1/IFNAR2 receptor complexes.

Several lines of evidence strongly implicate elevation of type I IFNs and increases in IFN-signaling in the pathogenesis of various autoimmune inflammatory diseases, such as systemic lupus erythematosus (SLE), rheumatoid arthritis (RA), systemic sclerosis (SSc), and Sjogren's syndrome (SS). Inhibitors of IFN signaling are currently being tested in a number of clinical trials (Chasset et al., 2021, Front. Pharmacol. 12:633821. doi: 10.3389/fphar.2021.633821).

Furthermore, type I IFNs are also upregulated as a response to oncolytic virus (OV) therapy. OVs are biotherapeutics that are effective against solid tumors. Unfortunately, upregulation of type I IFNs potentiates autoimmune side effects in patients, increases the resistance of tumor cells against oncolytic viruses, and reduces the therapeutic efficacy of this approach (EI-Sayes et al., 2022, Oncolytics 25:16-30). Preclinical studies showed that combination of OV therapy with inhibitors of IFN signaling was associated with improved treatment outcomes (EI-Sayes et al., 2022, Oncolytics 25:16-30; Ebrahimi et al., 2017, J Cell Biochem. 118(8)1994-9; Selman et al., 2018, Sci Transl Med. 10(425): eaao1613).

Nonetheless, the harm associated with treatments that indiscriminately inhibit type I IFN signaling can outweigh its benefits, given that IFNAR1/IFNAR2 receptor complexes are expressed throughout the body and systemic suppression of type I IFN signaling can weaken the body's immune responses, such as the ability to fight infections.

Thus, there is need in the art for novel therapies that specifically target and regulate improper or prolonged type I IFN signaling in specific cells.

### 4. SUMMARY

The present disclosure relates to novel molecular constructs, referred to herein as “IFN receptor antagonists,” which comprise an IFN moiety masked by an IFNAR1 moiety and are capable of robust IFN signal inhibition. Surprisingly, although comprising an IFN molecule, the IFN receptor antagonists described herein do not activate IFN signaling in a cell, but function exclusively as IFN signal blockers, competing for binding with exogenous IFN and preventing or significantly reducing IFN signaling in a cell via a Type I interferon receptor.

The IFN receptor antagonists generally comprise, in addition to an IFN moiety and an IFNAR1 moiety, an anchoring moiety (e.g., a targeting moiety), a separator moiety (e.g., an Fc domain), and, optionally, a linker connecting the IFNAR1 moiety to the IFN moiety. An IFN receptor antagonist may further comprise one or more linkers connecting one or more components (e.g., connecting a separator moiety to an IFNAR1 or IFN moiety). The anchoring moiety can bind to a target molecule present on the surface of a cell expressing a Type I interferon receptor. For example, an anchoring moiety may be a targeting moiety comprising an antigen-binding domain that can bind to such a target molecule. The separator moiety enables simultaneous binding of the anchoring moiety to the target molecule and the IFN moiety to the Type 1 interferon receptor on the cell. For example, the separator moiety may be an Fc domain.

Exemplary IFN moieties that can be used in the IFN receptor antagonists of the disclosure are described in Section 6.3.

Exemplary masking moieties that can be used in the IFN receptor antagonists of the disclosure are described in Section 6.4.

Linkers that can be used in the IFN receptor antagonists of the disclosure are described in Section 6.5.

Anchoring moieties that can be used in the IFN receptor antagonists of the disclosure are described in Section 6.6. Targeting moieties are described in Section 6.6.1, while targeting moiety formats are disclosed in Section 6.6.1.1. Separator moieties that can be incorporated into the IFN receptor antagonists of the disclosure are described in Section 6.7. Fc domains are described in Section 6.7.1.

Exemplary IFN receptor antagonists of the disclosure are described in Section 6.2 and numbered embodiments 1 to 90.

The disclosure further provides nucleic acids encoding the IFN receptor antagonists of the disclosure. The nucleic acids encoding the IFN receptor antagonists can be a single nucleic acid (e.g., a vector encoding all polypeptide chains of an IFN receptor antagonist) or a plurality of nucleic acids (e.g., two or more vectors encoding the different polypeptide chains of an IFN receptor antagonist). The disclosure further provides host cells and cell lines engineered to express the nucleic acids and IFN receptor antagonists of the disclosure. The disclosure further provides methods of producing an IFN receptor antagonist of the disclosure. Exemplary nucleic acids, host cells, and cell lines, and methods of producing an IFN receptor antagonist are described in Section 6.8 and numbered embodiments 91 to 93.

The disclosure further provides pharmaceutical compositions comprising the IFN receptor antagonists of the disclosure. Exemplary pharmaceutical compositions are described in Section 6.9 and numbered embodiments 94 and 95.

Further provided herein are methods of using the IFN receptor antagonists and the pharmaceutical compositions of the disclosure, e.g., for treating cancer or autoimmune conditions. Exemplary methods are described in Section 6.10 and numbered embodiments 96 to 112.

### 5. BRIEF DESCRIPTION OF THE FIGURES

FIGS. 1A-1F are cartoons representing targeted IFN receptor antagonists. FIG. 1A shows the components used to generate the masked IFN receptor antagonist (TM=targeting moiety; IFN=interferon; IFNAR1=Interferon alpha/beta receptor 1). FIGS. 1B-1F show the structures of the targeted IFN receptor antagonist constructs. As used in FIGS. 1A-1F, “IFN” refers generally to any IFN moiety (e.g., IFNα2b) and “IFNAR1” refers generally to any IFNAR1 moiety. Although targeting moieties in FIGS. 1A-1F are shown as Fabs, other targeting moieties (e.g., scFvs) can be used instead of Fabs.

FIGS. 2A-2B show the staining and bioassay activity profiles of non-targeted constructs comprising IFNAR1 (R1) masking moieties. FIG. 2A is a graph that shows the staining profiles of bivalent masked constructs. FIG. 2B is a graph that shows the in vitro activity of bivalent masked constructs.

FIGS. 3A-3I show the in vitro activity of exemplary targeted IFN receptor antagonists in the presence or absence of a constant amount of IFNα2b. In vitro activity of PDL1-targeted and isotype control (FIGS. 3A, 3D, and 3G), masked monovalent (FIGS. 3B, 3E, and 3H), and masked bivalent (FIGS. 3C, 3F, and 3I) constructs were assessed in PDL1 overexpressing (OE) KG1a/ISRE-Luc cells (FIGS. 3A-3C and 3G-3I) or PDL1 knockout (KO) KG1a/ISRE-Luc cells (FIGS. 3D-3F), titrated in the absence (FIGS. 3A-3C) or presence of 200 pM IFNα2b (FIGS. 3D-3I).

FIGS. 4A-4L show the in vitro activity of exemplary targeted IFN receptor antagonists and anti-IFNAR1 or anti-IFNAR2 antibodies in presence of a constant amount of IFNα2b or IFNβ. In vitro activity of PDL1-targeted and isotype control, masked monovalent, masked bivalent constructs and anti-IFNAR1 or anti-IFNAR2 antibodies were assessed in PDL1 KO (FIGS. 4A-4C and 4G-4I) or PDL1 OE (FIGS. 4D-4F and 4J-L) KG1a/ISRE-Luc cells, titrated in presence of 45 pM IFNα2b (FIGS. 4A-4F) or 95 pM IFNβ (FIGS. 4G-4L).

FIGS. 5A-5D show the in vitro cytoprotective activity of exemplary of unmasked control (FIG. 5A), targeted and isotype monovalent (FIG. 5B), and targeted and isotype bivalent (FIG. 5C) constructs, and anti-IFNAR1 and anti-IFNAR2 antibodies (FIG. 5D) in PDL1 OE KG1a/ISRE-Luc cells.

FIGS. 6A-6D show the in vitro cytoprotective activity of exemplary of unmasked control (FIG. 6A), targeted and isotype monovalent (FIG. 6B), and targeted and isotype bivalent (FIG. 6C) constructs, and anti-IFNAR1 and anti-IFNAR2 antibodies (FIG. 6D) in PDL1 KO KG1a/ISRE-Luc cells.

FIGS. 7A-7D show the in vitro cytoprotective activity of exemplary of unmasked control (FIG. 7A), targeted and isotype monovalent (FIG. 7B), and targeted and isotype bivalent (FIG. 7C) constructs, and anti-IFNAR1 and anti-IFNAR2 antibodies (FIG. 7D) in PDL1 OE KG1a/ISRE-Luc cells.

FIGS. 8A-8D show the in vitro cytoprotective activity of exemplary of unmasked control (FIG. 8A), targeted and isotype monovalent (FIG. 8B), and targeted and isotype bivalent (FIG. 8C) constructs, and anti-IFNAR1 and anti-IFNAR2 antibodies (FIG. 8D) in PDL1 KO KG1a/ISRE-Luc cells.

FIGS. 9A-9L show the in vitro activity of exemplary targeted IFN receptor antagonists in the presence or absence of a constant amount of IFNα2b (FIGS. 9A-9F) or IFNβ (FIGS. 9G-9L). In vitro activity of PDL1-targeted and isotype control (FIGS. 9A, 9D, 9G and 9J), masked monovalent (FIGS. 9B, 9E, 9H and 9K), and masked bivalent (FIGS. 9C, 9F, 9I and 9L) constructs were assessed in PDL1 knockout (KO) KG1a/ISRE-Luc cells (FIGS. 9A-9C and 9G-9I) or PDL1 overexpressing (OE) KG1a/ISRE-Luc cells (FIGS. 9D-9F and 9J-9L).

FIGS. 10A-10B show the effect of exemplary targeted and isotype IFN receptor antagonist constructs on IFNα2b-mediated IP10 release by monocyte-derived DCs (MoDCs) (FIG. 10A) and PDL1 expression in MoDCs (FIG. 10B).

FIGS. 11A-11F show the in vitro activity of exemplary IFN receptor antagonist constructs in the presence or absence of a constant amount of hIFNα2b or hIFNβ. In vitro activity of PDL1-targeted masked bivalent constructs and control constructs was assessed in PDL1 knockout (KO) KG1a/ISRE-Luc cells (FIGS. 11A-11C) and PDL1 overexpressing (OE) KG1a/ISRE-Luc cells (FIGS. 11D-11F).

FIGS. 12A-12F show the in vitro activity of exemplary IFN receptor antagonist constructs comprising universal type I interferon (uIFN) moieties in the presence or absence of a constant amount of hIFNα2b or hIFNβ. In vitro activity of isotype or PDL1-targeted masked bivalent constructs was assessed in PDL1 knockout (KO) KG1a/ISRE-Luc cells (FIGS. 12A, 12C, and 12E) and PDL1 overexpressing (OE) KG1a/ISRE-Luc cells (FIGS. 12B, 12D, and 12F).

FIGS. 13A-13F show the in vitro activity of exemplary IFN receptor antagonist constructs comprising universal type I interferon (uIFN) moieties in the presence or absence of a constant amount of hIFNα2b or hIFNβ. In vitro activity of isotype or EGFR-targeted masked bivalent constructs was assessed in KG1a/ISRE-Luc cells that do not express EGFR (“No EGFR”; FIGS. 13A, 13C, and 13E) and EGFR overexpressing (OE) KG1a/ISRE-Luc cells (FIGS. 13B, 13D, and 13F).

### 6. DETAILED DESCRIPTION

### 6.1. Definitions

As used herein, the following terms are intended to have the following meanings:

ABD chain, targeting moiety chain: Targeting moieties and antigen binding sites (ABD's) within them can exist as one (e.g., in the case of an scFv or scFab) polypeptide chain or form through the association of more than one polypeptide chains (e.g., in the case of a Fab or an Fv). As used herein, the terms “ABD chain” and “targeting moiety chain” refer to all or a portion of an ABD or targeting moiety that exists on a single polypeptide chain. The use of the term “ABD chain” or “targeting moiety chain” is intended for convenience and descriptive purposes only and does not connote a particular configuration or method of production. Further, the reference to an ABD or targeting moiety when describing an IFN receptor agonist encompasses an ABD chain or targeting moiety chain unless the context dictates otherwise. Thus, when describing an IFN receptor antagonist in which an Fc domain is operably linked to a targeting moiety, the Fc domain may be covalently linked directly or indirectly (e.g., via a linker) through a peptide bond to, e.g., (1) a first ABD or targeting moiety chain of a Fab or Fv (with the other components of the Fab or Fv on a second, associated ABD or targeting moiety chain) or (2) an ABD or targeting moiety chain containing an scFv or scFab.

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Anchoring Moiety: The term “anchoring moiety” as used herein refers to any molecule or portion thereof that can bind to a cell. Anchoring moieties contemplated herein include, but are not limited to, cell surface protein binding molecules (e.g. ligands and other protein binding partners, etc., such as those described in Section 6.6) and targeting moieties (e.g., antibodies and antigen-binding fragments, such as those described in Section 6.6.1). As used herein an anchoring moiety “for” a particular cell means that the anchoring moiety is capable of binding to the particular cell. The binding need not be selective or specific.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antagonistic: The terms “antagonistic” or “antagonist” as used herein with reference to an IFN receptor antagonist refer to the ability to reduce signaling, activation, or activity of a Type I interferon receptor in the presence of IFNα2b. In some embodiments, an IFN receptor antagonist is a molecule which reduces interferon signaling by at least 10% as measured by an activity assay as described in Section 9.1.4 in the presence of IFNα2b.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding domain: The term “antigen-binding domain” or “ABD” as used herein refers to a portion of an antibody or antibody fragment (e.g., a targeting moiety) that has the ability to bind to an antigen non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136).

Associated: The term “associated” in the context of an IFN receptor antagonist refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional IFN receptor antagonist. Examples of associations that might be present in an IFN receptor antagonist of the disclosure include (but are not limited to) associations between Fc domains to form an Fc region (homodimeric or heterodimeric as described in Section 6.7), associations between VH and VL regions in a Fab or Fv, and associations between CH1 and CL in a Fab.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, esophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), AI-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Constant domain: The terms “constant domain” refers to a CH1, CH2, CH3 or CL domain of an immunoglobulin.

The term “CH1 domain” refers to the heavy chain constant region linking the variable domain to the hinge in a heavy chain constant domain. In some embodiments, the term “CH1 domain” refers to the region of an immunoglobulin molecule spanning amino acids 118 to 215 (EU numbering). The term “CH1 domain” encompasses wildtype CH1 domains as well as variants thereof (e.g., non-naturally-occurring CH1 domains or modified CH1 domains). For example, the term “CH1 domain” includes wildtype IgG1, IgG2, IgG3 and IgG4 CH1 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH1 domains include CH1 domains with mutations that modify a biological activity of an antibody, such as ADCC, CDC or half-life.

The term “CH2 domain” refers to the heavy chain constant region linking the hinge to the CH3 domain in a heavy chain constant domain. In some embodiments, the term “CH2 domain” refers to the region of an immunoglobulin molecule spanning amino acids 238 to 340 (EU numbering). The term “CH2 domain” encompasses wildtype CH2 domains as well as variants thereof (e.g., non-naturally-occurring CH2 domains or modified CH2 domains). For example, the term “CH2 domain” includes wildtype IgG1, IgG2, IgG3 and IgG4 CH2 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH2 domains include CH2 domains with mutations that modify a biological activity of an antibody, such as ADCC, CDC, purification, dimerization and half-life.

The term “CH3 domain” refers to the heavy chain constant region that is C-terminal to the CH2 domain in a heavy chain constant domain. In some embodiments, the term “CH3 domain” refers to the region of an immunoglobulin molecule spanning amino acids 341 to 447 (EU numbering). The term “CH3 domain” encompasses wildtype CH3 domains as well as variants thereof (e.g., non-naturally-occurring CH3 domains or modified CH3 domains). For example, the term “CH3 domain” includes wildtype IgG1, IgG2, IgG3 and IgG4 CH3 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH3 domains include CH3 domains with mutations that modify a biological activity of an antibody, such as ADCC, CDC, purification, dimerization and half-life.

The term “CL domain” refers to the constant region of an immunoglobulin light chain. The term “CL domain” encompasses wildtype CL domains (e.g., kappa or lambda light chain constant regions) as well as variants thereof (e.g., non-naturally-occurring CL domains or modified CL domains). For example, the term “CL domain” includes wildtype kappa and lambda constant domains and variants thereof having 1, 2, 3, 4, 5, 1-3, 1-5, 3-5 and/or at most 5, 4, 3, 2, or 1 mutations, e.g., substitutions, deletions and/or additions.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case, it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. In some embodiments an Fc domain comprises a CH2 domain followed by a CH3 domain, with or without a hinge region N-terminal to the CH2 domain. The term “Fc region” refers to the region of formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Half Antibody: The term “half antibody” refers to a molecule that comprises at least one Fc domain and can associate with another molecule comprising an Fc domain through, e.g., a disulfide bridge or molecular interactions. A half antibody can be composed of one polypeptide chain or more than one polypeptide chains (e.g., the two polypeptide chains of a Fab). An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABD. Yet another example of a half antibody is a polypeptide comprising an scFv domain, a CH2 domain and a CH3 domain.

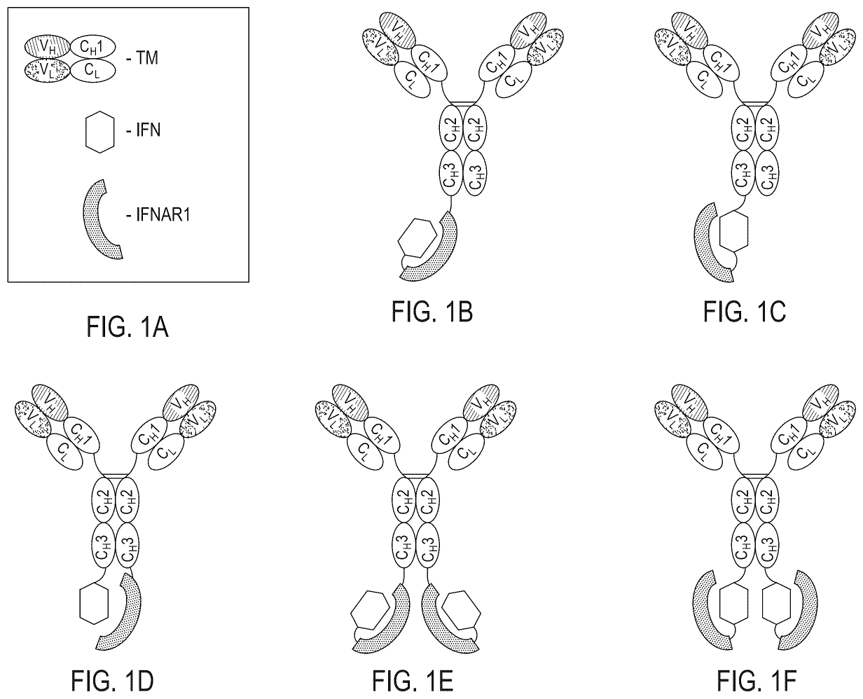

The IFN receptor antagonists of the disclosure typically comprise two half antibodies, each comprising an IFN moiety masked by one or two receptor moieties, e.g., IFN masking moieties. The one or two masking moieties can be in the same half antibody as the IFN moiety or can be in the other half antibody from the IFN moiety, as exemplified in the embodiments illustrated in FIGS. 1B-1G and set forth in Table 2. Exemplary positions of linkers (e.g., non-cleavable linkers) in the half antibodies are shown in Table 1. One or both half antibodies in the IFN receptor antagonists may further comprise a targeting moiety, such as an scFv or Fab. Exemplary IFN receptor antagonists comprising targeting moieties are illustrated in FIGS. 1B-1G and set forth in Table 2.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host Cell or Recombinant Host Cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing an IFN receptor antagonist of the disclosure, a host cell is preferably a cell line of mammalian origin or mammalian-like characteristics, such as monkey kidney cells (COS, e.g., COS-1, COS-7), HEK293), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., derivatives that grow at higher density than the original cell lines and/or glycan profile modified derivatives and and/or site-specific integration site derivatives.

Interferon: The term “interferon” as used herein refers to a full-length interferon or to a modified interferon, for example a truncated and/or mutant interferon. In some embodiments, the modified interferon is attenuated as compared to the corresponding wildtype interferon (e.g., retains less than 50%, less than 40%, less than 30%, less than 20%, or less than 10%, less than 1%, less than 0.1% or less than 0.05% activity in an in vitro luciferase reporter assay as described in Section 9.1.5). In some embodiments, the modified interferon is attenuated by a range bounded by any two of the foregoing values, e.g., 0.05%-50%, 0.1%-20%, 0.1%-10%, 0.05%-5%, 1%-20%, and so on and so forth. In other embodiments the modified interferon substantially retains the biological activity of the corresponding wildtype interferon (e.g., retains at least 50% activity in an in vitro luciferase reporter assay as described in Section 9.1.5). Interferons include Type I interferons (e.g., interferon-α and interferon-β) as well as Type II interferons (e.g., interferon-γ). The term “interferon” also encompasses a synthetic or engineered protein having the biological activity of a wildtype interferon (e.g., having at least 50% activity in an in vitro luciferase reporter assay as described in Section 9.1.5), such as, for example, a universal Type I interferon as described in Section 6.3.1.

Linker: The term “linker” as used herein refers to a connecting peptide between two moieties. For example, a linker can connect an IFN moiety and an IFN masking moiety.

Non-cleavable linker: A non-cleavable linker as used herein refers to a peptide whose amino acid sequence lacks a substrate sequence for a protease. Example non-cleavable linker are described in Section 6.5.

Oncolytic virus: The term “oncolytic virus” refers to a virus that replicates in tumor cells. These include viruses that naturally preferentially replicate and accumulate in tumor cells, such as poxviruses, and viruses that have been engineered to do so. Some oncolytic viruses can kill a tumor cell following infection of the tumor cell. For example, an oncolytic virus can cause death of the tumor cell by lysing the tumor cell or inducing cell death of the tumor cell. Exemplary oncolytic viruses include, but are not limited to, poxviruses, herpesviruses, adenoviruses, adeno-associated viruses (AAV), lentiviruses, retroviruses, rhabdoviruses, papillomaviruses, vesicular stomatitis virus (VSV), measles virus, Newcastle disease virus, picornavirus, Sindbis virus, papillomavirus, parvovirus, reovirus, coxsackievirus. In certain aspects, an oncolytic virus of the disclosure is VSV. Oncolytic viruses and their use to treat cancer are described further in, for example, Chiocca and Rabkin Cancer Immunol Res (2014) 2(4): 295-300.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a IFN receptor antagonist of the disclosure, separate components (e.g., an Fc domain and an IFN moiety) can be operably linked directly or through peptide linker sequences. In the context of a nucleic acid encoding a fusion protein, such as a half antibody of an IFN receptor antagonist of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Recognize: The term “recognize” as used herein refers to an antibody or antibody fragment (e.g., a targeting moiety) that finds and interacts (e.g., binds) with its epitope.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably non-cleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single-chain Fv” or “scFv” as used herein refers to ABDs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL and be arranged in the N- to C-terminal order VH-VL or VL-VH, typically separated by a linker, for example a linker as set forth in Table E.

Separator moiety: The term “separator moiety” as used herein refers to an amino acid sequence which, as a component of an IFN receptor antagonist comprising an anchoring moiety and an IFN moiety, provides sufficient spatial separation of the anchoring moiety and the IFN moiety to allow their concurrent binding to the same cell, e.g., a cell as described in Section 9.1.3. In some embodiments, a separator moiety is a polypeptide at least about 100 amino acids in length. In particular embodiments, a separator moiety of the disclosure comprises an Fc domain or a fragment thereof.

Specifically (or selectively) binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subiect: The term “subject” includes human and non-human animals. Non-human animals include all vertebrates, e.g., mammals and non-mammals, such as non-human primates, sheep, dog, cow, chickens, amphibians, and reptiles. In preferred embodiments, the subject is human.

Target Molecule: The term “target molecule” as used herein refers to any biological molecule (e.g., protein, carbohydrate, lipid or combination thereof) expressed on a cell surface or in the extracellular matrix that can be specifically bound by a targeting moiety in an IFN receptor antagonist of the disclosure.

Targeting Moiety: The term “targeting moiety” as used herein refers to any molecule or binding portion (e.g., an immunoglobulin or an antigen binding fragment) thereof that can bind to a cell surface molecule on a cell to which an IFN receptor antagonist of the disclosure is to be localized, for example on cells expressing a Type I interferon receptor (e.g., on lymphocytes implicated in an autoimmune condition). The targeting moiety can also have a functional activity in addition to localizing an IFN receptor antagonist to a particular site. For example, a targeting moiety that binds to a checkpoint inhibitor such as PDL1 can also exhibit anti-tumor activity, for example by inhibiting PD1/PDL1 signaling.

T-Cell Antigen, TCA: The term “T-cell antigen” or “TCA” refers to a molecule (typically a protein, carbohydrate, lipid or some combination thereof) that is expressed on the surface of a T-lymphocyte and is useful for the preferential targeting of a pharmacological agent to a particular site. In some embodiments, the site is cancer tissue and/or the T-cell antigen is a tumor reactive lymphocyte antigen, a cell surface molecule of tumor or viral lymphocytes, or a checkpoint inhibitor expressed on a T-lymphocyte.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen, TAA: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (TSAs).

Treat, Treatment, Treating: As used herein, the terms “treat”, “treatment” and “treating” refer to the reduction or amelioration of the progression, severity and/or duration of a disorder, e.g., an inflammatory disorder, an autoimmune disorder, or a proliferative disorder, or the amelioration of one or more symptoms (preferably, one or more discernible symptoms) of a disorder resulting from the administration of one or more IFN receptor antagonists of the disclosure. In some embodiments, the disorder is an inflammatory, autoimmune, or proliferative disorder and the terms “treat”, “treatment” and “treating” refer to the amelioration of at least one measurable physical parameter of an inflammatory, autoimmune, or proliferative disorder, not necessarily discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count in a proliferative disorder.

Universal Light Chain, ULC: The term “universal light chain” or “ULC” as used herein refers to a light chain variable region (VL) that can pair with more than on heavy chain variable region (VL). In the context of a targeting moiety, the term “universal light chain” or “ULC” refers to a light chain polypeptide capable of pairing with the heavy chain region of the targeting moiety and also capable of pairing with other heavy chain regions. ULCs can also include constant domains, e.g., a CL domain of an antibody. Universal light chains are also known as “common light chains.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. IFN Receptor Antagonists

The present disclosure relates to IFN receptor antagonists comprising an anchoring moiety, a separator moiety, a Type I interferon (IFN) moiety, and a Type I interferon alpha receptor 1 (IFNAR1) moiety.

IFN receptor antagonists of the disclosure generally comprise one or more anchoring moieties that bind to a cell expressing a Type I interferon receptor. An anchoring moiety may be designed to specifically bind to a particular cell type that expresses a Type I interferon receptor, depending on the desired indication or use. Accordingly, in some embodiments, IFN receptor antagonists of the disclosure comprise one or more targeting moieties, e.g., antigen binding domains of antibodies, that target the IFN receptor antagonists to a cell that expresses a Type I interferon receptor, e.g., cancer cells or lymphocytes implicated in an autoimmune disorder.

IFN receptor antagonists of the disclosure generally comprise one or more separator moieties that separate the anchoring moiety from the IFN moiety and IFNAR1 moiety. A separator moiety may be any molecule or polypeptide which enables simultaneous binding of the anchoring moiety and the IFN moiety to the same cell. In some cases, a separator moiety is a multimerization moiety which enables multimerization (e.g., dimerization) of two or more individual components. Accordingly, in embodiments where the IFN receptor antagonists are dimers, the IFN receptor antagonists are generally composed of two half antibodies, comprising a pair of multimerization domains, such as Fc domains that associate to form an Fc region (typically comprising hinge sequences). In the IFN receptor antagonists of the disclosure, the two half antibodies together comprise at least one IFN moiety but may include two or more IFN moieties. The IFN moieties in the IFN receptor antagonists may each be masked by an interferon alpha receptor 1 (IFNAR1) moiety.

Exemplary IFN receptor antagonists are illustrated in FIGS. 1B-1G.

Table 1 below describes exemplary half antibodies that can be incorporated into the IFN receptor antagonists of the disclosure. As evident from Table 1, each half antibody may include one or more polypeptide chains. For convenience when describing combinations of half antibodies in the IFN receptor antagonists of the disclosure, each half antibody described in Table 1 is often referred to herein as an “Exemplary Monomer”.

TABLE 1

| Exemplary | | |
|---|---|---|
| Monomer | Nomenclature | Polypeptide Chain(s) |
| 1 | Fc | Chain 1: Fc domain |
| 1T | TM-Fc | Chain 1: Targeting moiety (or targeting |
| | | moiety component)-optional linker 1-Fc |
| | | domain |
| | | Chain 2 (optional): Targeting moiety |
| | | component (e.g., VL or VL-CL) |
| 2 | Fc-IFN | Chain 1: Fc domain-optional linker |
| | | 1-IFN moiety |
| 2T | TM-Fc-IFN | Chain 1: Targeting moiety (or targeting |
| | | moiety component)-optional linker 1-Fc |
| | | domain-optional linker 2-IFN moiety |
| | | Chain 2 (optional): Targeting moiety |
| | | component (e.g., VL or VL-CL) |
| 3 | IFN-Fc | Chain 1: IFN moiety-optional linker |
| | | 1-Fc domain |
| 4A | Fc-IFNAR1 or | Chain 1: Fc domain-optional linker 1- |
| | Fc-R1 | IFNAR1 moiety |
| 4AT | TM-Fc-IFNAR1 | Chain 1: Targeting moiety (or targeting |
| | or TM-Fc-R1 | moiety component)-optional linker 1-Fc |
| | | domain-optional linker 2-IFNAR1 moiety |
| | | Chain 2 (optional): Targeting moiety |
| | | component (e.g., VL or VL-CL) |
| 5A | Fc-IFNAR1-IFN | Chain 1: Fc domain-optional linker 1- |
| | or Fc-R1-IFN | IFNAR1 moiety-optional linker 2-IFN |
| | | moiety |
| 5AT | TM-Fc-IFNAR1- | Chain 1: Targeting moiety (or targeting |
| | IFN or | moiety component)-optional linker 1-Fc |
| | TM-Fc-R1-IFN | domain-optional linker 2-IFNAR1 |
| | | moiety-optional linker 3-IFN moiety |
| | | Chain 2 (optional): Targeting moiety |
| | | component (e.g., VL) |
| 6A | Fc-IFN-IFNAR1 | Chain 1: Fc domain-optional linker 1-IFN |
| | or Fc-IFN-R1 | moiety-optional linker 2-IFNAR1 moiety |
| 6AT | TM-Fc-IFN- | Chain 1: Targeting moiety (or targeting |
| | IFNAR1 or | moiety component)-optional linker 1-Fc |
| | TM-FC-IFN-R1 | domain-optional linker 2-IFN moiety- |
| | | optional linker 3-IFNAR1 moiety |
| | | Chain 2 (optional): Targeting moiety |
| | | component (e.g., VL or VL-CL) |
| 7A | Fc-IFNAR1 or | Chain 1: Fc domain-optional linker 1- |
| | Fc-R1 | IFNAR1 moiety |
| 7AT | TM-Fc-IFNAR1 | Chain 1: Targeting moiety (or targeting |
| | or TM-Fc-R1 | moiety component)-optional linker 1-Fc |
| | | domain-optional linker 2-IFNAR1 moiety |
| | | Chain 2 (optional): Targeting moiety |
| | | component (e.g., VL or VL-CL) |
| 8A | Fc-IFNAR1- | Chain 1: Fc domain-optional linker 1- |
| | IFN-IFNAR1 | IFNAR1 moiety-optional linker 2-IFN |
| | | moiety-optional linker 3-IFNAR1 moiety |
| 9A | IFNAR1-IFN-Fc | Chain 1: IFNAR1 moiety-optional |
| | or R1-IFN-Fc | linker 1-IFN moiety-optional |
| | | linker 2-Fc domain |

In some embodiments, all linkers in the IFN receptor antagonists are non-cleavable. Exemplary linkers are described in Section 6.5. The Fc domains in the polypeptide chains described in Table 1 preferably comprise a hinge domain as set forth in Section 6.7.1.3.

In some embodiments, the anchoring moiety is a targeting moiety (e.g., as described in Section 6.6.1) that binds to a cell surface protein. In other embodiments, the anchoring moiety is a cell surface protein binding molecule. In some embodiments, the separator moiety is an Fc domain (e.g., as described in Section 6.7.1). Without intending to be bound by theory, the inventors believe that in this configuration, the anchoring moiety anchors the IFN receptor antagonist to the cell, thereby enabling binding of the IFNAR1-masked IFN moiety to the Type I interferon receptor on the cell and inhibition of IFN signaling.

Table 2 below shows additional Exemplary Monomer pairings that can be utilized in the IFN receptor antagonists of the disclosure. The IFN receptor antagonists identified in Table 2 comprise two targeting moieties.

TABLE 2

| | | | Illustrative |
|---|---|---|---|
| | Left Half-Antibody | Right Half-Antibody | FIG. |
| 1. | Exemplary Monomer 5AT | Exemplary Monomer 1T | 1B |
| 2. | Exemplary Monomer 6AT | Exemplary Monomer 1T | 1C |
| 3. | Exemplary Monomer 2T | Exemplary Monomer 7AT | 1D |
| 4. | Exemplary Monomer 5AT | Exemplary Monomer 5AT | 1E |
| 5. | Exemplary Monomer 6AT | Exemplary Monomer 6AT | 1F |

Sequence and length of hinge and linker sequences can be varied, as can the sequence of the IFN moiety (containing either the full-length or N- and/or C-terminal truncated IFN sequences as well as amino acid substitutions). Exemplary IFN moieties are described in Section 6.3 and include IFNα- and IFNβ-based moieties as described in Sections 6.3.1 and 6.3.2 below as well as other Type I IFN-based moieties as described in Section 6.3.1. Exemplary IFNAR1 moieties are disclosed in Section 6.4. Exemplary linker and hinge sequences are disclosed in Sections 6.5 and 6.7.1.3, respectively. Exemplary targeting moieties are disclosed in Section 6.6. Exemplary Fc domains, including Fc domains suitable for heterodimerization when the two half antibodies of an IFN receptor antagonist are not identical, are described in Section 6.7.

### 6.3. The IFN Moiety

There are two major classes of IFNs: Type I (IFN-α subtypes, IFN-β, etc.) and Type II (IFN-γ). Additional IFNs (IFN-like cytokines; IFN-λ subtype) have also been identified.

The IFN moiety of the disclosure may comprise any wild type or modified (e.g., truncated and/or mutant) IFN or IFN-like cytokine sequence but preferably is a Type I IFN moiety. Type I IFNs bind a heterodimeric plasma membrane receptor IFNAR made of IFNAR1 and IFNAR2 that is ubiquitously expressed in all nucleated cells. Ligand binding is initiated by high-affinity receptor subunit IFNAR2 (Piehler et al., 2012, Immunological Reviews, doi.org/10.1111/imr.12001). As such, Type I IFNs are able to act on virtually all cells of the body. Sixteen Type I interferon subtypes have been identified, which vary in their intrinsic variability in affinity to IFNAR2 and activity.

In some embodiments, the Type I IFN moiety is an interferon-α (IFN α) moiety. In other embodiments, Type I IFN moiety is an interferon-β (IFNβ) moiety.

In other embodiments, the Type I IFN moiety is an interferon-ω (IFNω), interferon-ε (IFNε) or interferon-κ (IFNκ) moiety.

The Type I IFN moiety may comprise a sequence that varies from a wild-type IFN sequence by one or more mutations, e.g., substitutions, deletions, or insertions. Substitutions that attenuate IFN activity by reducing receptor binding may suitably be used. Amino acids with N- or C-terminal deletions (or truncations) may also be used, e.g., a truncation of 1, 2, 3, 4, 5, 6, 7, 8, 9, 10, 11, 12, 13, 14 or 15 amino acids from the N- and/or C-termini of a mature Type I IFN.

Further details of exemplary Type I IFN moieties are provided below.

### 6.3.1. Interferon-α Moieties

The IFNα gene is a member of the alpha interferon gene cluster on chromosome 9. The encoded cytokine is a member of the Type I interferon family that is produced in response to viral infection as a key part of the innate immune response with potent antiviral, antiproliferative and immunomodulatory properties. IFNα refers to a family of proteins, with at least 15 known subtypes of human IFNα. The major subtypes identified are IFNα1, IFNα2, IFNα8, IFNα10, IFNα14 and IFNα21.

The IFNα1 gene has two allelic variants: IFNα 1a and IFNα1b. The amino acid sequence of human IFNα1a is assigned UniProtKB accession number P01562, reproduced below with the signal peptide underlined:

(SEQ ID NO: 1)
MASPFALLMV LVVLSCKSSC SLGCDLPETH SLDNRRTLML
LAQMSRISPS SCLMDRHDFG FPQEEFDGNQ FQKAPAISVL
HELIQQIFNL FTTKDSSAAW DEDLLDKFCT ELYQQLNDLE
ACVMQEERVG ETPLMNADSI LAVKKYFRRI TLYLTEKKYS
PCAWEVVRAE IMRSLSLSTN LQERLRRKE

The human IFNα1b gene differs the IFNα1a allelic variant by one base change in the coding region, leading to a single change in amino acid sequence (Val114 instead of Ala114 in the mature protein, corresponding to Val137 instead of Ala137 in the full-length polypeptide).

There are three allelic variants of IFNα2 alleles, IFNα2a, IFNα2b and IFNα2c. Allele IFNα2b is the predominant allele while allele IFNα2a is less predominant and IFNα2c only a minor allelic variant. The amino acid sequence of human IFNα2 is assigned UniProtKB accession number P01563. The sequence of the IFNα2b allele is reproduced below with the signal peptide underlined:

(SEQ ID NO: 2)
MALTFALLVA LLVLSCKSSC SVGCDLPQTH SLGSRRTLML
LAQMRRISLF SCLKDRHDFG FPQEEFGNQF QKAETIPVLH
EMIQQIFNLF STKDSSAAWD ETLLDKFYTE LYQQLNDLEA
CVIQGVGVTE TPLMKEDSIL AVRKYFQRIT LYLKEKKYSP
CAWEVVRAEI MRSFSLSTNL QESLRSKE

IFNα2b has an arginine (R) at position 23 of the mature protein while IFNα2a has a lysine (K). Thus, in some embodiments, the IFNα2 moiety has an arginine at the position corresponding to position 23 of the mature protein. In other embodiments, the IFNα2 moiety has a lysine at the position corresponding to position 23 of the mature protein.

In various aspects, the IFNα moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFNα1a, IFNα1b, and/or IFNα2b, IFNα2a, or IFNα2c or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of mature IFNα1a, IFNα1b, and/or IFNα2b, IFNα2a, or IFNα2c).

In some embodiments, the IFNα moiety has one or more amino acid substitutions, e.g., substitutions that alter IFNAR binding and/or agonism. Exemplary substitutions are found in WO 2013/107791, U.S. Pat. No. 8,258,263, WO2007/000769A2, WO2008/124086, WO2010/030671, WO2018/144999A1, and WO2015/007520, WO 2013/059885, WO2020156467A1, WO2021/126929A1. In some embodiments, the IFNα moiety comprises:
a) one or more substitutions selected from L15A, A19W, R22A, R23A, L26A, F27A, L30A, L30V, K31A, D32A, R33K or R33A or R33Q, H34A, D35A, Q40A, H57Y, E58N, Q61S, F64A, N65A, T69A, L80A, D82E, Y85A, T861, Y89A, D114R or D114A, L117A, R120A or R120E or R120K, K121E, R125A, K133A, K134A, R144A, A145G orA145M, M148A, R149A, R149K, S152A, L153A, N156A; and/or
b) one or more substitutions at amino acids 57-89 and 159-165 described in WO2007000769A2; and/or
c) one or more amino acid substitutions at 9, 17, 47, 65, 66, 117, 123, 128, 147, and 157 to alanine, glycine, or threonine described in WO2021126929A1.

The amino acid positions of the foregoing substitutions are given with reference to mature IFNα2b.

In further embodiments, the IFNα moiety comprises one or more amino acid substitutions set forth in Table 3. Table 3 sets forth IFNα substitutions identified by reference to the amino acid position within the sequence of IFNα2.

TABLE 3
Exemplary IFNα mutations
(with reference to the sequence of mature IFNα2)

| IFNα sequence mutation(s) | Source/impact of substitution(s) |
|---|---|
| L15A | WO2018014068A9 |
| R22A | WO2018014068A9 |
| R23A | WO2018014068A9 |
| S25A | WO2018014068A9 |
| L26A | Thomas et al., 2011, Cell, |
| | 146(4): 621-632 |
| F27A | Thomas et al., 2011, Cell, |
| | 146(4): 621-632 |
| L30A | Thomas et al., 2011, Cell, |
| | 146(4): 621-632 |
| L30V | WO2018014068A9 |
| K31A | WO2018014068A9 |
| D32A | WO2018014068A9 |
| R33K | WO2018014068A9 |
| R33Q | WO2018014068A9 |
| R33A | WO2013059885A2 |
| H34A | WO2018014068A9 |
| D35E | WO2016201337A1 |
| Q40A | WO2018014068A9 |
| H57A | Thomas et al., 2011, Cell, |
| | 146(4): 621-632 |
| H57S | WO2013134138A1 |
| E58A | Thomas et al., 2011, Cell, |
| | 146(4): 621-632 |
| Q61A | WO2016201337A1 |
| Q90A | Piehler et al., 2012, |
| | Immunological Reviews, |
| | doi.org/10.1111/imr.12001 |
| E96A | Piehler et al., 2012, |
| | Immunological Reviews, |
| | doi.org/10.1111/imr.12001 |
| D114R | WO2018014068A9 |
| L117A | WO2018014068A9 |
| R120A | WO2018014068A9 |
| R120E | WO2018014068A9 |
| Q124R | WO2022015711A1 |
| R125A | WO2018014068A9 |
| R125E | WO2018014068A9 |
| K131A | WO2018014068A9 |
| E132A | WO2018014068A9 |
| K133A | WO2018014068A9 |
| K134A | WO2018014068A9 |
| L135A | WO2013134138A1 |
| R144A | WO2022015711A1 |
| R144D | WO2018014068A9 |
| R144E | WO2018014068A9 |
| R144G | WO2018014068A9 |
| R144H | WO2018014068A9 |
| R144I | WO2018014068A9 |
| R144K | WO2018014068A9 |
| R144L | WO2013059885A2 |
| R144N | WO2018014068A9 |
| R144Q | WO2018014068A9 |
| R144S | WO2013059885A2 |
| R144T | WO2013059885A2 |
| R144V | WO2018014068A9 |
| R144Y | WO2013059885A2 |
| A145D | WO2013059885A2 |
| A145E | WO2018014068A9 |
| A145G | WO2018014068A9, |
| | WO2022015711A1 |
| A145H | WO2013059885A2 |
| A145I | WO2018014068A9 |
| A145K | WO2013059885A2 |
| A145L | WO2018014068A9 |
| A145M | WO2018014068A9 |
| A145N | WO2018014068A9 |
| A145Q | WO2018014068A9 |
| A145S | WO2018014068A9 |
| A145T | WO2018014068A9 |
| A145V | WO2018014068A9 |
| A145Y | WO2013059885A2 |
| M148A | WO2018014068A9 |
| R149A | WO2022015711A1, |
| | Piehler et al., 2012, |
| | Immunological Reviews, |
| | doi.org/10.1111/imr.12001 |
| R149K | Piehler et al., 2012, |
| | Immunological Reviews, |
| | doi.org/10.1111/imr.12001 |
| S152A | Piehler et al., 2012, |
| | Immunological Reviews, |
| | doi.org/10.1111/imr.12001, |
| | WO2022015711A1 |
| L153A | WO2018014068A9 |
| N156A | WO2018014068A9 |
| R162A | WO2016201337A1 |
| E165D | WO2016201337A1 |
| L30A, H57Y, E58N, Q61S | WO2018014068A9 |
| R33A, H57Y, E58N, Q61S | WO2013059885A2 |
| H57S, E58S, Q61S | Alter IFNα-IFNAR1 |
| | binding affinity at Site 1 |
| H57Y, E58N, Q61S | WO2007000769A2 |
| N65A, L80A, Y85A, Y89A | WO2018014068A9 |
| N65A, L80A, Y85A, Y89A, | WO2018014068A9 |
| D114A | |
| N65A, L80A, Y85A, Y89A, | WO2018014068A9 |
| L174A | |
| N65A, L80A, Y85A, Y89A, | WO2018014068A9 |
| R120A | |
| Y85A, Y89A, D114A | WO2018014068A9 |
| Q90A, R120A | Piehler et al., 2012, |
| | Immunological Reviews, |
| | doi.org/10.1111/imr.12001 |
| D114A, R120A | WO2018014068A9 |
| L117A, R120A | WO2018014068A9 |
| L117A, R120A, K121A | WO2018014068A9 |
| R120A, K121A | WO2018014068A9 |
| R120E, K121E | WO2018014068A9 |
| R144A, H57Y, E58N, Q61S | WO2013059885A2 |
| M148A, H57Y, E58N, Q61S | WO2018014068A9 |
| R149A, R162A | WO2013134138A1 |
| L153A, H57Y, E58N, Q61S | WO2018014068A9 |
| Deletion of residues L161 to | WO2018014068A9 |
| E165 | |

In some embodiments, the IFNα moiety comprises an amino acid sequence comprising the amino acid substitution R33A or R33K, Q90A, E96A, R120A, R120E, A145M, R149A or R149K, S152A, or any combination of two or more of the foregoing, e.g., Q90A+R120A or A145M+R149K.

The sequences of exemplary IFNα moieties that can be utilized in the IFN receptor antagonists of the disclosure are set forth in Table 4 below:

TABLE 4
Exemplary IFNα Moieties

| Construct | Sequence | SEQ ID NO: |
|---|---|---|
| IFNα1a | CDLPETHSLDNRRTLMLLAQMSRISPSSCLMDRHDFGFPQEEFD | 3 |
| | GNQFQKAPAISVLHELIQQIFNLFTTKDSSAAWDEDLLDKFCTE | |
| | LYQQLNDLEACVMQEERVGETPLMNADSILAVKKYFRRITLYLT | |
| | EKKYSPCAWEVVRAEIMRSLSLSTNLQERLRRKE | |
| IFNα1b | CDLPETHSLDNRRTLMLLAQMSRISPSSCLMDRHDFGFPQEEFD | 4 |
| | GNQFQKAPAISVLHELIQQIFNLFTTKDSSAAWDEDLLDKFCTE | |
| | LYQQLNDLEACVMQEERVGETPLMNVDSILAVKKYFRRITLYLT | |
| | EKKYSPCAWEVVRAEIMRSLSLSTNLQERLRRKE | |
| IFNα2a | CDLPQTHSLGSRRTLMLLAQMRKISLESCLKDRHDFGFPQEEFG | 5 |
| | NQFQKAETIPVLHEMIQQIFNLFSTKDSSAAWDETLLDKFYTEL | |
| | YQQLNDLEACVIQGVGVTETPLMKEDSILAVRKYFQRITLYLKE | |
| | KKYSPCAWEVVRAEIMRSFSLSTNLQESLRSKE | |
| IFNα2b | CDLPQTHSLGSRRTLMLLAQMRRISLFSCLKDRHDFGFPQEEFG | 6 |
| | NQFQKAETIPVLHEMIQQIFNLFSTKDSSAAWDETLLDKFYTEL | |
| | YQQLNDLEACVIQGVGVTETPLMKEDSILAVRKYFQRITLYLKE | |
| | KKYSPCAWEVVRAEIMRSFSLSTNLQESLRSKE | |
| IFNα2b | CDLPQTHSLGSRRTLMLLAQMRRISLFSCLKDAHDFGFPQEEFG | 7 |
| (R33A) | NQFQKAETIPVLHEMIQQIENLESTKDSSAAWDETLLDKFYTEL | |
| | YQQLNDLEACVIQGVGVTETPLMKEDSILAVRKYFQRITLYLKE | |
| | KKYSPCAWEVVRAEIMRSESLSTNLQESLRSKE | |
| IFNα2b | CDLPQTHSLGSRRTLMLLAQMRRISLFSCLKDKHDFGFPQEEFG | 8 |
| (R33K) | NQFQKAETIPVLHEMIQQIFNLFSTKDSSAAWDETLLDKFYTEL | |
| | YQQLNDLEACVIQGVGVTETPLMKEDSILAVRKYFQRITLYLKE | |
| | KKYSPCAWEVVRAEIMRSFSLSTNLQESLRSKE | |
| IFNα2b | CDLPQTHSLGSRRTLMLLAQMRRISLESCLKDRHDFGFPQEEFG | 9 |
| (Q90A) | NQFQKAETIPVLHEMIQQIFNLFSTKDSSAAWDETLLDKFYTEL | |
| | YAQLNDLEACVIQGVGVTETPLMKEDSILAVRKYFQRITLYLKE | |
| | KKYSPCAWEVVRAEIMRSFSLSTNLQESLRSKE | |
| IFNα2b | CDLPQTHSLGSRRTLMLLAQMRRISLESCLKDRHDFGFPQEEFG | 10 |
| (E96A) | NQFQKAETIPVLHEMIQQIFNLFSTKDSSAAWDETLLDKFYTEL | |
| | YQQLNDLAACVIQGVGVTETPLMKEDSILAVRKYFQRITLYLKE | |
| | KKYSPCAWEVVRAEIMRSFSLSTNLQESLRSKE | |
| IFNα2b | CDLPQTHSLGSRRTLMLLAQMRRISLESCLKDRHDFGFPQEEFG | 11 |
| (R120A) | NQFQKAETIPVLHEMIQQIFNLFSTKDSSAAWDETLLDKFYTEL | |
| | YQQLNDLEACVIQGVGVTETPLMKEDSILAVAKYFQRITLYLKE | |
| | KKYSPCAWEVVRAEIMRSFSLSTNLQESLRSKE | |
| IFNα2b | CDLPQTHSLGSRRTLMLLAQMRRISLESCLKDRHDFGFPQEEFG | 12 |
| (A145M) | NQFQKAETIPVLHEMIQQIFNLESTKDSSAAWDETLLDKFYTEL | |
| | YQQLNDLEACVIQGVGVTETPLMKEDSILAVRKYFQRITLYLKE | |
| | KKYSPCAWEVVRMEIMRSFSLSTNLQESLRSKE | |
| IFNα2b | CDLPQTHSLGSRRTLMLLAQMRRISLESCLKDRHDFGFPQEEFG | 13 |
| (R149A) | NQFQKAETIPVLHEMIQQIENLESTKDSSAAWDETLLDKFYTEL | |
| | YQQLNDLEACVIQGVGVTETPLMKEDSILAVRKYFQRITLYLKE | |
| | KKYSPCAWEVVRAEIMASFSLSTNLQESLRSKE | |
| IFNα2b | CDLPQTHSLGSRRTLMLLAQMRRISLESCLKDRHDFGFPQEEFG | 14 |
| (R149K) | NQFQKAETIPVLHEMIQQIFNLFSTKDSSAAWDETLLDKFYTEL | |
| | YQQLNDLEACVIQGVGVTETPLMKEDSILAVRKYFQRITLYLKE | |
| | KKYSPCAWEVVRAEIMKSFSLSTNLQESLRSKE | |
| IFNα2b | CDLPQTHSLGSRRTLMLLAQMRRISLESCLKDRHDFGFPQEEFG | 15 |
| (S152A) | NQFQKAETIPVLHEMIQQIFNLESTKDSSAAWDETLLDKFYTEL | |
| | YQQLNDLEACVIQGVGVTETPLMKEDSILAVRKYFQRITLYLKE | |
| | KKYSPCAWEVVRAEIMRSFALSTNLQESLRSKE | |
| ΔN-IFN-ΔC | THSLGSRRTLMLLAQMRRISLFSCLKDRHDFGFPQEEFGNQFQK | 16 |
| | AETIPVLHEMIQQIENLESTKDSSAAWDETLLDKFYTELYQQLN | |
| | DLEACVIQGVGVTETPLMKEDSILAVRKYFQRITLYLKEKKYSP | |
| | CAWEVVRAEIMRSFSLSTNLQ | |
| IFN-ΔC | CDLPQTHSLGSRRTLMLLAQMRRISLESCLKDRHDFGFPQEEFG | 17 |
| | NQFQKAETIPVLHEMIQQIFNLFSTKDSSAAWDETLLDKFYTEL | |
| | YQQLNDLEACVIQGVGVTETPLMKEDSILAVRKYFQRITLYLKE | |
| | KKYSPCAWEVVRAEIMRSFSLSTNLQ | |
| IFNα2b | CDLPQTHSLGSRRTLMLLAQMRRISLESCLKDRHDFGFPQEEFG | 138 |
| (R120E) | NQFQKAETIPVLHEMIQQIFNLESTKDSSAAWDETLLDKFYTEL | |
| | YQQLNDLEACVIQGVGVTETPLMKEDSILAVEKYFQRITLYLKE | |
| | KKYSPCAWEVVRAEIMRSFSLSTNLQESLRSKE | |
| IFNα2b | CDLPQTHSLGSRRTLMLLAQMRRISLFSCLKDRHDFGFPQEEFG | 139 |
| (8CT) | NQFQKAETIPVLHEMIQQIFNLESTKDSSAAWDETLLDKFYTEL | |
| | YQQLNDLEACVIQGVGVTETPLMKEDSILAVEKYFQRITLYLKE | |
| | KKYSPCAWEVVRAEIMRSESLSTNLQKRLKSKE | |

### 6.3.2. Interferon-β Moieties

Interferon-β (IFNβ) is a cytokine that is naturally produced by the immune system in response to biological and chemical stimuli. IFNβ is a glycosylate, secreted monomer having a molecular weight of around 22 kDa that is produced in large quantities by fibroblasts and as such it is also known as fibroblast interferon. IFNβ binds to the IFNAR receptor composed of the IFNAR1 and IFNAR2 dimers to induce signaling via the JAK/STAT pathway and other pathways. IFNβ can also function by binding to IFNAR1 alone and signal independently of the Jak-STAT pathways (Ivashkiv and Donlin, 2014, Nat Rev Immunol. 14(1):36-49).

IFNβ contains 5 α-helices designated A (ynllgflqrssnfqcqkll (SEQ ID NO:101)), B (kedaaltiyemlqnifaif (SEQ ID NO:102)), C (etivenllanvyhqinhlktvleekl (SEQ ID NO:103)), D (sslhlkryygrilhylka (SEQ ID NO:104)), and E (hcawtivrveilrnfyfinrlt (SEQ ID NO:105)). The five α-helices are interconnected by loops of 2-28 residues designated AB, BC, CD and DE loops. It has been reported that the A helix in the AB loop and the E helix in the DE loop are involved in the binding of IFNβ to the IFNAR receptor.

Two types of IFNβ have been described: Interferon-β1 (IFNβ1) and Interferon-β3 (IFNβ3) (Schirmer and Neumann, 2019. Cytokines. In: Nijkamp and Parnham's Principles of Immunopharmacology. Springer, Cham.).

The amino acid sequence of human IFNβ precursor is listed under GenBank: accession number AAA36040.1 and reproduced below (with the signal peptide underlined):

(SEQ ID NO: 18)
MTNKCLLQIA LLLCFSTTAL SMSYNLLGFL QRSSNFQCQK
LLWQLNGRLE YCLKDRMNFD IPEEIKQLQQ FQKEDAALTI
YEMLQNIFAI FRQDSSSTGW NETIVENLLA NVYHQINHLK
TVLEEKLEKE DFTRGKLMSS LHLKRYYGRI LHYLKAKEYS
HCAWTIVRVE ILRNFYFINR LTGYLRN

In various aspects, the IFNβ moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFNβ1 or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFNβ1).

In various embodiments, the IFNβ moiety comprises one or more amino acid substitutions and/or deletions as compared to IFNβ1. In some embodiments, the substitution is a C17S (with reference to the mature IFNβ1) and the deletions are one of the C-terminal truncations described in US 2009/0025106 A1as IFN-ΔI, IFNA2, IFNA3, IFNA4, IFNA5, IFNA6, IFN-Δ7, IFN-Δδ, IFNA9, and IFN-ΔI O.

### 6.3.1. Universal Type I Interferon

In certain aspects, the Type I IFN moiety is a Universal Type I IFN (also referred to as human IFN-alpha hybrid protein, recombinant human universal type I IFN, or simply “uIFN”), which is a recombinant IFNα moiety constructed from IFNα A and IFNα D. uIFN exhibits bioactivity across multiple species.

The amino acid sequence of Universal Type I IFN (uIFN) is reproduced below:

(SEQ ID NO: 137)
CDLPQTHSLGSRRTLMLLAQMRKISLFSCLKDRHDFGFPQEEFGNQFQK
AETIPVLHEMIQQIFNLFTTKDSSAAWDEDLLDKFCTELYQQLNDLEAC
VMQEERVGETPLMNVDSILAVKKYFRRITLYLTEKKYSPCAWEVVRAEI
MRSLSLSTNLQERLRRKE

In some embodiments, the IFN moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of uIFN (SEQ ID NO:137) or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of uIFN).

### 6.3.2. Other Type I Interferons

In certain aspects, the Type I IFN moiety is other than an IFNα or IFNβ moiety, e.g., an interferon-ω (IFNω), interferon-ε (IFNε) or interferon-κ (IFNκ) moiety.

Human IFNω is identified by UniProt accession no. P05000 and the IFNω1 allele has the amino acid sequence set forth below, with the signal sequence underlined:

(SEQ ID NO: 19)
MALLFPLLAALVMTSYSPVGSLGCDLPQNHGLLSRNTLVLLHQMRRISP
FLCLKDRRDFRFPQEMVKGSQLQKAHVMSVLHEMLQQIFSLFHTERSSA
AWNMTLLDQLHTGLHQQLQHLETCLLQVVGEGESAGAISSPALTLRRYF
QGIRVYLKEKKYSDCAWEVVRMEIMKSLFLSTNMQERLRSKDRDLGSS

In various aspects, the IFNω moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFNω1 or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFNω1).

Human IFNε is identified by UniProt accession no. Q86WN2 and has the amino acid sequence set forth below, with the signal sequence underlined:

(SEQ ID NO: 20)
MIIKHFFGTVLVLLASTTIFSLDLKLIIFQQRQVNQESLKLLNKLQTLS
IQQCLPHRKNFLLPQKSLSPQQYQKGHTLAILHEMLQQIFSLFRANISL
DGWEENHTEKFLIQLHQQLEYLEALMGLEAEKLSGTLGSDNLRLQVKMY
FRRIHDYLENQDYSTCAWAIVQVEISRCLFFVFSLTEKLSKQGRPLNDM
KQELTTEFRSPR

In various aspects, the IFNε moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFNε or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFNε).

Human IFNκ is identified by UniProt accession no. Q9P0W0 and has the amino acid sequence set forth below, with the signal sequence underlined:

(SEQ ID NO: 21)
MSTKPDMIQKCLWLEILMGIFIAGTLSLDCNLLNVHLRRVTWQNLRHLS
SMSNSFPVECLRENIAFELPQEFLQYTQPMKRDIKKAFYEMSLQAFNIF
SQHTFKYWKERHLKQIQIGLDQQAEYLNQCLEEDKNENEDMKEMKENEM
KPSEARVPQLSSLELRRYFHRIDNFLKEKKYSDCAWEIVRVEIRRCLYY
FYKFTALFRRK

In various aspects, the IFNκ moiety comprises an amino acid sequence having at least about 90%, at least about 91%, at least about 92%, about at least 93%, at least about 94%, at least about 95%, at least about 96%, at least about 97%, at least about 98%, at least about 99% or 100% sequence identity to the amino acid sequence of mature IFNκ or a fragment thereof having a truncation of up to 15 amino acids at its N- and/or C-terminus (e.g., a truncation of 1, 2, 3, 4, 5, 6, 7, 8, 9, 10, 11, 12, 13, 14 or 15 amino acids from the N- and/or C-termini of IFNκ).

### 6.4. IFN Masking Moieties

The present disclosure provides IFN receptor antagonists with the IFN moiety masked by one or more receptor moieties. All human type I interferons bind to a cell surface receptor (IFN alpha receptor, IFNAR; also “Type I interferon receptor”) which is a heterodimer consisting of two transmembrane proteins, IFNAR1 and IFNAR2 (see, e.g., Novick et al., 1994, Cell 77:391). As described herein, IFN receptor antagonists of the disclosure, which comprise an IFN moiety masked by IFNAR1, are capable of inhibiting IFN signaling in a cell expressing a Type I interferon receptor. In contrast, analogous constructs comprising an IFN moiety masked by IFNAR2 are unable to inhibit IFN signaling. Thus, in particular embodiments, the masking moiety is an IFNAR1 moiety.

As used herein, “inhibiting IFN signaling” describes a reduction of signaling, activation, or activity of a Type I interferon receptor in a cell. For example, an IFN receptor antagonist of the disclosure may be said to inhibit IFN signaling where the molecule reduces interferon signaling by at least 10% as measured by an activity assay as described in Section 9.1.5.

Exemplary IFNAR1 moieties are disclosed in Section 6.4.1.

### 6.4.1. IFNAR1 Moiety

IFNAR1 is the lower affinity IFN receptor and belongs to the type II spiral-type cytokine receptors. It includes an extracellular domain that is composed of 4 type III fibronectin domains referred to as “subdomains” (SDs), a transmembrane domain and an intracellular domain of 100 amino acids. The four subdomains of IFNAR1 fold into domain 1 (SD1+SD2) and domain 2 (SD3+SD4).

The sequence of human IFNAR1 has the UniProt identifier P17181. The sequence of human IFNAR1 is reproduced below:

(SEQ ID NO: 22)
MMVVLLGATTLVLVAVAPWVLSAAAGGKNLKSPQKVEVDIIDDNFILRW
NRSDESVGNVTFSFDYQKTGMDNWIKLSGCQNITSTKCNFSSLKLNVYE
EIKLRIRAEKENTSSWYEVDSFTPFRKAQIGPPEVHLEAEDKAIVIHI
SPGTKDSVMWALDGLSFTYSLVIWKNSSGVEERIENIYSRHKIYKLSPE
TTYCLKVKAALLTSWKIGVYSPVHCIKTTVENELPPPENIEVSVQNQN
YVLKWDYTYANMTFQVQWLHAFLKRNPGNHLYKWKQIPDCENVKTTQCV
FPQNVFQKGIYLLRVQASDGNNTSFWSEEIKFDTEIQafllppvfnirs
lsdsfhiyigapkqsgntpviqdypliyeiifwentsnaerkiiekktd
vtvpnlkpltvycvkarahtmdeklnkssvfsdavcektkpgNTSKIWL
IVGICIALFALPFVIYAAKVFLRCINYVFFPSLKPSSSIDEYFSEQPLK
NLLLSTSEEQIEKCFIIENISTIATVEETNQTDEDHKKYSSQTSQDSGN
YSNEDESESKTSEELQQDEV

The signal sequence (single underline) corresponds to amino acids 1-27, the SD1 domain (bold) corresponds to amino acids 28-127, the SD2 domain (double underline) corresponds to amino acids 128-227, the SD3 domain (italics) corresponds to amino acids 231-329, the SD4 domain (lowercase) corresponds to amino acids 330-432, and the extracellular domain corresponds to amino acids 28-436 of the full length human IFNAR1 protein reproduced above.

An IFNAR1 moiety is an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity, or 100% sequence identity, to an IFN-binding portion of a mammalian, e.g., human, IFNAR1. In some embodiments, the IFN-binding portion comprises the SD2 and SD3 domains. In various aspects, the IFN-binding portion comprises (i) only the SD2 and SD3 domains; (ii) the SD1, SD2 and SD3 domains; (iii) the SD2, SD3 and SD4 domains; (iv) the SD1, SD2, SD3 and SD4 domains; or (v) the entire extracellular domain of IFNAR1.

### 6.5. Linkers

In certain aspects, the present disclosure provides IFN receptor antagonists in which two or more components of an IFN receptor antagonist are connected to one another by a peptide linker. By way of example and not limitation, linkers can be used to connect a separator moiety (e.g., an Fc domain) and an anchoring moiety (e.g., a targeting moiety), different domains within an anchoring moiety (e.g., VH and VL domains in an scFv), a separator domain (e.g., an Fc domain) and an IFN or IFNR1 moiety, or an IFN moiety and an IFNR1 moiety.

Preferably, all linkers in the IFN receptor are non-cleavable linkers (NCLs).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the peptide linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the IFN receptor antagonists of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GₙS (SEQ ID NO:106) or SGₙ (SEQ ID NO:107), where n is an integer from 1 to 10, e.g., 1, 2, 3, 4, 5, 6, 7, 8, 9 or 10. In one embodiment, the non-cleavable linker is or comprises a monomer or multimer of repeat of G₄S (SEQ ID NO: 108) e.g., (GGGGS)ₙ (SEQ ID NO:108).

Polyglycine non-cleavable linkers can suitably be used in the IFN receptor antagonists of the disclosure. In some embodiments, a peptide non-cleavable linker comprises two consecutive glycines (2Gly), three consecutive glycines (3Gly), four consecutive glycines (4Gly) (SEQ ID NO:109), five consecutive glycines (5Gly) (SEQ ID NO:110), six consecutive glycines (6Gly) (SEQ ID NO:111), seven consecutive glycines (7Gly) (SEQ ID NO: 112), eight consecutive glycines (8Gly) (SEQ ID NO: 113) or nine consecutive glycines (9Gly) (SEQ ID NO:114).

Exemplary linker sequences are set forth in Table E below.

TABLE E
Exemplary Peptide Linker Sequences

| Linker Sequence | Designation | SEQ ID NO |
|---|---|---|
| GGGGSLALGPGGGGGSLALGPGGGGGSLALGPGGS | NCL1 | 23 |
| GGGGSGGGGSGGGGSGGGGSGGGGS | NCL2 | 24 |
| (GGGGS)ₙ, optionally where n = 1-10, e.g., 1, 2, 3, 4, 5, 6, 7, 8, | NCL3 | 25 |
| 9, or 10 | | |
| (GGGS)ₙ, optionally where n = 1-10, e.g., 1, 2, 3, 4, 5, 6, 7, 8, | NCL4 | 26 |
| 9, or 10 | | |
| (GGS)ₙ, optionally where n = 1-10, e.g., 1, 2, 3, 4, 5, 6, 7, 8, | NCL5 | 27 |
| 9, or 10 | | |
| (GS)ₙ, optionally where n = 1-10, e.g., 1, 2, 3, 4, 5, 6, 7, 8, 9, | NCL6 | 28 |
| or 10 | | |
| (GSGGS)ₙ, optionally where n = 1-10, e.g., 1, 2, 3, 4, 5, 6, 7, 8, | NCL7 | 29 |
| 9, or 10 | | |
| ADAAP | NCL8 | 30 |
| ADAAPTVSIFP | NCL9 | 31 |
| ADAAPTVSIFPP | NCL10 | 32 |
| AKTTAP | NCL11 | 33 |
| AKTTAPSVYPLAP | NCL12 | 34 |
| AKTTPKLEEGEFSEARV | NCL13 | 35 |
| AKTTPKLGG | NCL14 | 36 |
| AKTTPP | NCL15 | 37 |
| AKTTPPSVTPLAP | NCL16 | 38 |
| ASTKGP | NCL17 | 39 |
| ASTKGPSVFPLAPASTKGPSVFPLAP | NCL18 | 40 |
| EGKSSGSGSESKST | NCL19 | 41 |
| GEGESGEGESGEGES | NCL20 | 42 |
| GEGESGEGESGEGESGEGES | NCL21 | 43 |
| GEGGSGEGGSGEGGS | NCL22 | 44 |
| GENKVEYAPALMALS | NCL23 | 45 |
| GGEGSGGEGSGGEGS | NCL24 | 46 |
| GGGESGGEGSGEGGS | NCL25 | 47 |
| GGGESGGGESGGGES | NCL26 | 48 |
| GGGGSGGGGS | NCL27 | 49 |
| GGGGSGGGGSGGGGS | NCL28 | 50 |
| GGGGSGGGGSGGGGSGGGGS | NCL29 | 51 |
| GGGKSGGGKSGGGKS | NCL30 | 52 |
| GGGKSGGKGSGKGGS | NCL31 | 53 |
| GGGS | NCL32 | 54 |
| GGGSG | NCL33 | 55 |
| GGKGSGGKGSGGKGS | NCL34 | 56 |
| GGSG | NCL35 | 57 |
| GGSGG | NCL36 | 58 |
| GGSGGGGSG | NCL37 | 59 |
| GGSGGGGSGGGGS | NCL38 | 60 |
| GHEAAAVMQVQYPAS | NCL39 | 61 |
| GKGGSGKGGSGKGGS | NCL40 | 62 |
| GKGKSGKGKSGKGKS | NCL41 | 63 |
| GKGKSGKGKSGKGKSGKGKS | NCL42 | 64 |
| GKPGSGKPGSGKPGS | NCL43 | 65 |
| GKPGSGKPGSGKPGSGKPSGS | NCL44 | 66 |
| GPAKELTPLKEAKVS | NCL45 | 67 |
| GSAGSAAGSGEF | NCL46 | 68 |
| GSGGG | NCL47 | 69 |
| GSGSG | NCL48 | 70 |
| GSS | NCL49 | N/A |
| GSSG | NCL50 | 72 |
| GSSGGSGGSG | NCL51 | 73 |
| GSSGGSGGSGG | NCL52 | 74 |
| GSSGGSGGSGGS | NCL53 | 75 |
| GSSGGSGGSGGSG | NCL54 | 76 |
| GSSGGSGGSGGSGGGS | NCL55 | 77 |
| GSSGGSGGSGS | NCL56 | 78 |
| GSSGT | NCL57 | 79 |
| GSSSG | NCL58 | 80 |
| GSTSGSGKPGSGEGSTKG | NCL59 | 81 |
| GTAAAGAGAAGGAAAGAAG | NCL60 | 82 |
| GTSGSSGSGSGGSGSGGGG | NCL61 | 83 |
| IRPRAIGGSKPRVA | NCL62 | 84 |
| KESGSVSSEQLAQFRSLD | NCL63 | 85 |
| KTTPKLEEGEFSEAR | NCL64 | 86 |
| PRGASKSGSASQTGSAPGS | NCL65 | 87 |
| QPKAAP | NCL66 | 88 |
| QPKAAPSVTLFPP | NCL67 | 89 |
| RADAAAA(G4S)₄ | NCL68 | 90 |
| RADAAAAGGPGS | NCL69 | 91 |
| RADAAP | NCL70 | 92 |
| RADAAPTVS | NCL71 | 93 |
| SAKTTP | NCL72 | 94 |
| SAKTTPKLEEGEFSEARV | NCL73 | 95 |
| SAKTTPKLGG | NCL74 | 96 |
| STAGDTHLGGEDFD | NCL75 | 97 |
| TVAAP | NCL76 | 98 |
| TVAAPSVFIFPP | NCL77 | 99 |
| TVAAPSVFIFPPTVAAPSVFIFPP | NCL78 | 100 |

In certain aspects, the IFN receptor antagonist of the disclosure may comprise a polypeptide chain comprising, in an N- to C-terminal orientation, a targeting moiety (or targeting moiety chain), a hinge domain, and an Fc domain. Thus, the hinge domain can be said to constitute a type of linker. Exemplary hinge domains are set forth in Section 6.7.1.3.

### 6.6. Anchoring Moiety

The IFN receptor antagonists of the disclosure preferably include one or more anchoring moieties. The incorporation of anchoring moieties permits the anchoring of the IFN receptor antagonist to a cell expressing a Type I interferon receptor, enabling binding of the IFN moiety to the Type I interferon receptor and inhibition of IFN signaling.

As a component of an IFN receptor antagonist, an anchoring moiety may be any molecule that binds to a molecule on the surface of a cell expressing a Type I interferon receptor. In certain aspects, an anchoring moiety is a “cell surface protein binding molecule,” which refers to any molecule capable of binding to a protein present or expressed on the surface of a cell. Certain anchoring moieties contemplated herein include, for example, ligands, receptors or ligand-binding portions thereof that bind to a cell surface ligand, cell surface protein-binding antibodies or fragments thereof, and lipid-binding antibodies or fragments thereof.

In certain aspects, an anchoring moiety binds to a molecule on the surface of a particular cell or cell type, both allowing for binding of the IFN moiety to the Type I interferon receptor and also targeting the IFN moiety to a particular cell type (e.g., cancer cell, immune cell, etc.). Accordingly, in some embodiments, an anchoring moiety of the present disclosure is a targeting moiety.

Exemplary anchoring moieties of the present disclosure are described further below.

### 6.6.1. Targeting Moiety

In particular embodiments, an anchoring moiety of the present disclosure is a targeting moiety. It is anticipated that any type of target molecule present at or capable of driving the IFN receptor antagonist to a cell expressing a Type I interferon receptor may be targeted by the IFN receptor antagonists of the disclosure. In some embodiments, the IFN receptor antagonists are intended to treat cancer, e.g., by reducing a local autoimmune response associated with oncolytic virotherapy. Accordingly, the targeting molecule can be a tumor-associated antigen targeting molecule, a checkpoint inhibitor targeting molecule, or a molecule targeting a cell surface molecule of tumor or viral lymphocytes. In some other embodiments, the IFN receptor antagonists are intended to treat autoimmune inflammatory diseases, such as systemic lupus erythematosus (SLE), rheumatoid arthritis (RA), e.g., by reducing a local or tissue-specific autoimmune response. Accordingly, the targeting molecule can be an immune cell targeting molecule, for example a T-cell targeting molecule, a B-cell targeting molecule, a dendritic cell-targeting molecule, an antigen-presenting cell targeting molecule, or a natural killer cell targeting molecule.

The target molecules recognized by the targeting moieties of the IFN receptor antagonists of the disclosure are generally found, for example, on the surfaces of activated T cells, on the surfaces of tumor cells, on the surfaces of dendritic or other antigen-presenting cells, on the surfaces of natural killer (NK) cells, on the surfaces of virus-infected cells, or on the surfaces of other diseased cells. In various embodiments, the target molecule is a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen. The skilled artisan would recognize that the foregoing categories of target molecules are not mutually exclusive and thus a given target molecule may fall into more than one of the foregoing categories of target molecules. For example, some molecules may be considered both TCAs and checkpoint inhibitors.

Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Other target molecules are cell surface molecules of tumor or viral lymphocytes, for example T-cell co-stimulatory proteins such as CD27, CD28, 4-1BB (CD137), OX40, CD30, CD40, ICOS, lymphocyte function-associated antigen-1 (LFA-1), CD2, CD7, LIGHT, NKG2C, and B7-H3.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In some embodiments, the target molecule is PDL1.

Exemplary immune cells that may be targeted include, but are not limited to, T cells (e.g., cytotoxic T cells, regulatory T cells, etc.), B cells, dendritic cells, natural killer (NK) cells, NKT cells, macrophages, and other antigen presenting cells.

In certain embodiments, the target molecules are on the surfaces of dendritic cells or other antigen-presenting cells, such as XCR1, Clec9a, CD1c, CD11c, CD14, PDL1, macrophage mannose receptor (CD206), and DEC-205.

In further embodiments, the target molecules are on the surfaces of natural killer (NK) cells such as CD335, CD38, CD2, NKG2D, NKp44, NKp30, CD16, LFA-1, CD27, KIR, NKH1A, and NKp46.

Suitable targeting moiety formats are described in Section 6.6.1.1. The targeting moiety is preferably an antigen binding moiety, for example an antibody or an antigen-binding portion of an antibody, e.g., an scFv, as described in Section 6.6.1.1.2 or a Fab, as described in Section 6.6.1.1.1.

In some embodiments, the targeting moieties target the exemplary target molecules set forth in Table F below, together with references to exemplary antibodies or antibody sequences upon which the targeting moiety can be based.

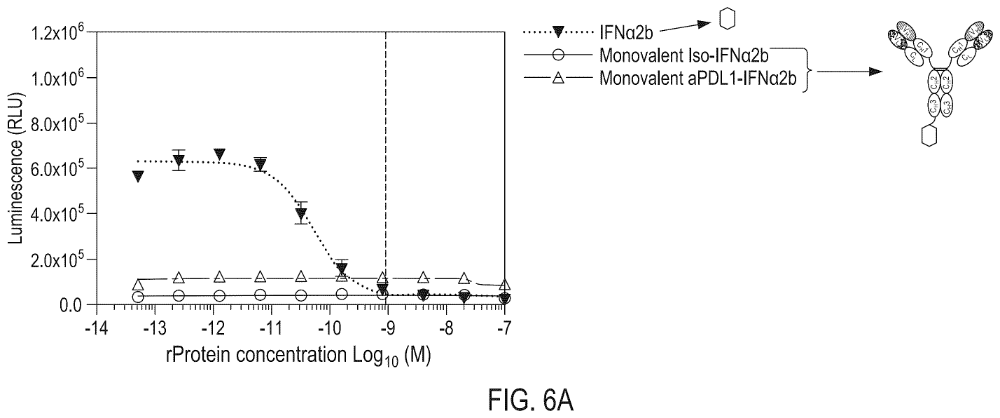

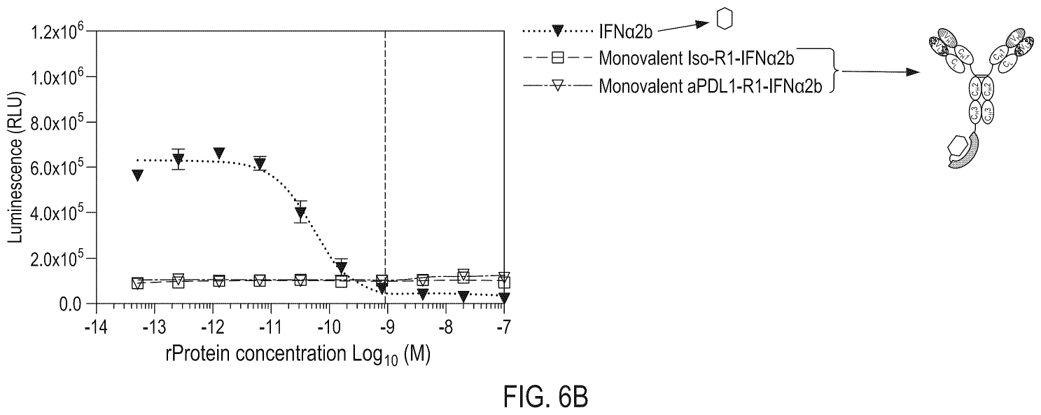

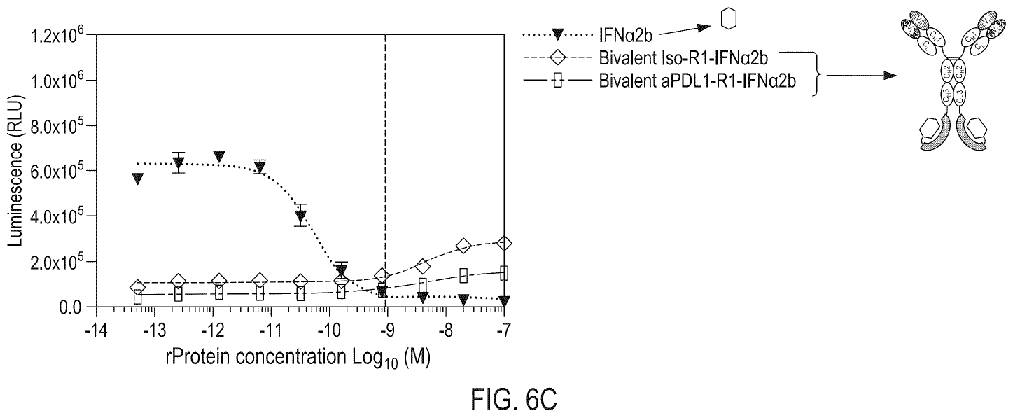

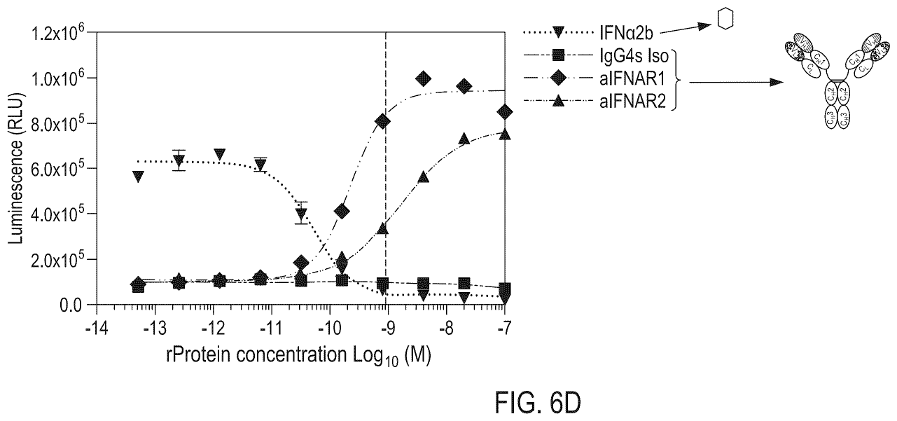

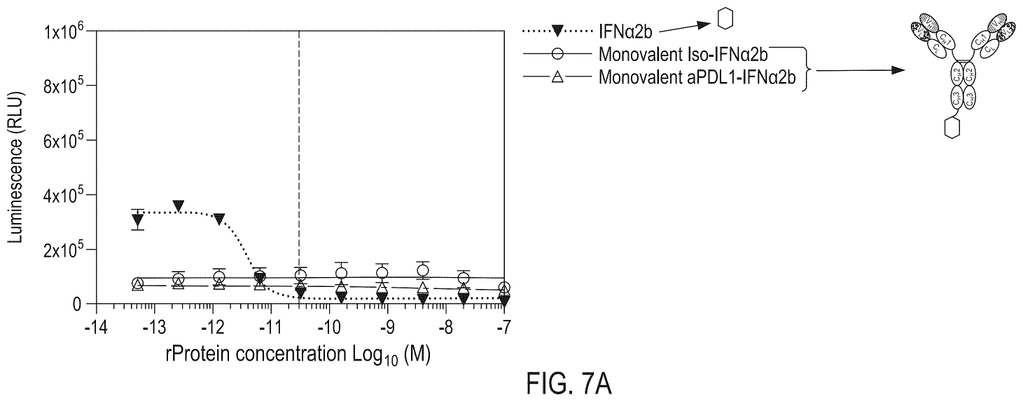

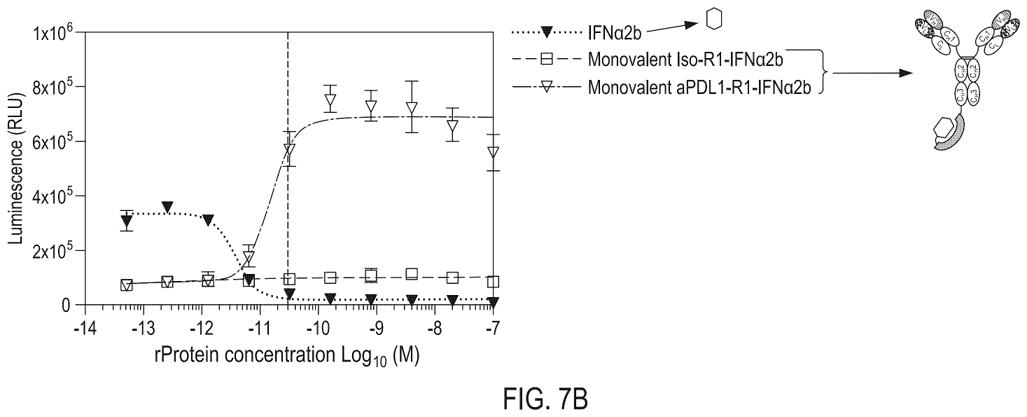

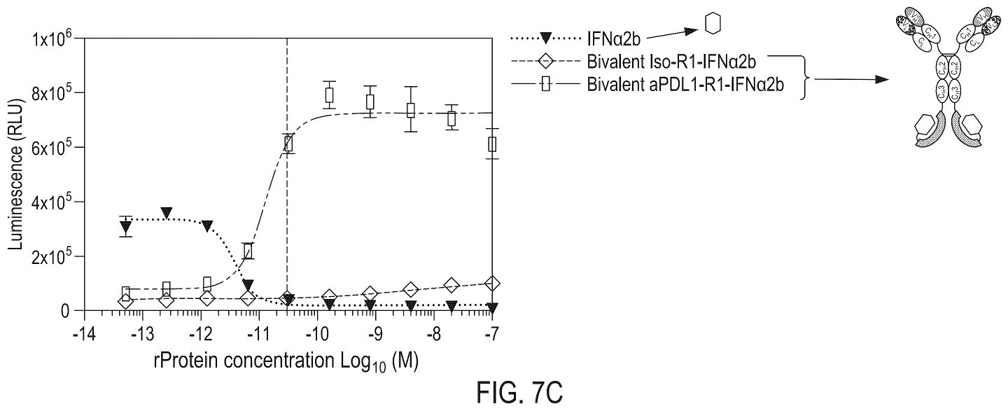

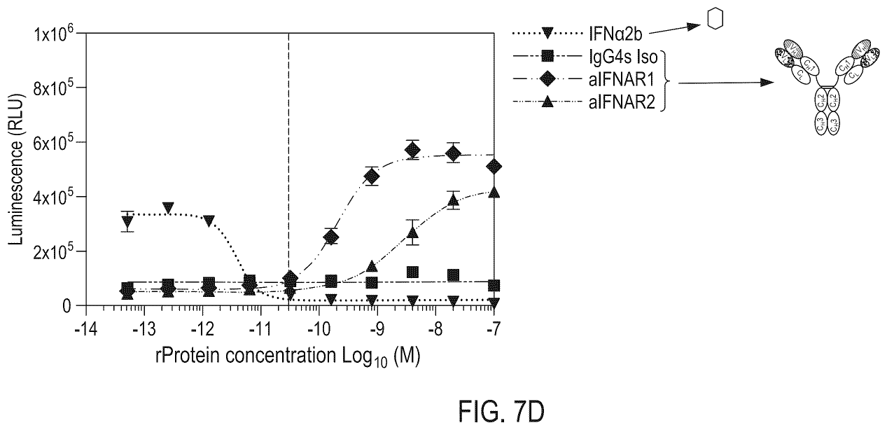

TABLE F
Exemplary Target Molecules

| Target | Antibody Name and/or Binding Sequences |
|---|---|
| 1-92-LFA-3 | Amevive ™ (alefacept) |
| 5T4 | GEN1044 |
| Activin | Bimagrumab |
| Receptor | VH: SEQ ID NOs: 107, 109 of |
| Type II | U.S. Pat. No. 8,388,968 B2 |
| | VL: SEQ ID NOs: 93, 95 of |
| | U.S. Pat. No. 8,388,968 B2 |
| B7-H3 | Obrindatamab (MGD009) |
| B7-H3 | Enoblituzumab (MGA271) |
| (CD276) | |
| B7-H3 | MGC018 |
| (CD276) | |
| B7-H3 | MGA012 |
| (CD276) | |
| B7-H3 | 8H9 |
| (CD276) | |
| B7-H3 | VH: the VH sequence of the heavy chain of SEQ ID |
| (CD276) | NO: 21, 26 or 31 of US 2021/0171641 A1. |
| | VL: the VL sequence of the light chain of SEQ ID NO: |
| | 20, 22 or 30 of US 2021/0171641 A1. |
| B7-H3 | VH: the VH sequence of the heavy chain of SEQ ID |
| (CD276) | NO: 21, 29 or 37 of US 2019/0002563 A1. |
| | VL: the VL sequence of the light chain of SEQ ID NO: |
| | 17, 25 or 33 of US 2019/0002563 A1. |
| B7-H3 | VH: the VH sequence of the heavy chain of SEQ ID |
| (CD276) | NO: 146, 147 or 148 of U.S. Pat. No. 10,640,563. |
| | VL: the VL sequence of the light chain of SEQ ID NO: |
| | 143, 144 or 145 of U.S. Pat. No. 10,640,563. |
| BAFF/B | Benlysta ™ (velimumab) |
| Lymphocyte | |
| Stimulator | |
| BAFF/B | VH: amino acids 1-123 of SEQ ID NO: 327 of |
| Lymphocyte | U.S. Pat. No. 7,138,501 |
| Stimulator | VL: amino acids 139-249 of SEQ ID NO: 327 of |
| | U.S. Pat. No. 7,138,501. |
| BAFF/B | VH: amino acids 1-126 of SEQ ID NO: 1321 of |
| Lymphocyte | U.S. Pat. No. 7,605,236; |
| Stimulator | VL: amino acids 143-251 of SEQ ID NO: 1049 of |
| | U.S. Pat. No. 7,605,236. |
| BAFF/B | Belimumab |
| Lymphocyte | |
| Stimulator | |
| BCMA | VH: the VH sequence of the heavy chain of |
| | SEQ ID NO. 126 of US 2021/0206865 A1 |
| | VL: the VL sequence of the light chain of |
| | SEQ ID NO. 129 or SEQ ID NO. 132 of |
| | US 2021/0206865 A1 |
| CA125 | Igobumab |
| CA125 | OvaRex ™ (oregobumab) |
| Cadherin | The antibodies described in US Pub. No. US |
| | 2006/0039915. |
| N-cadherin | An antibody that binds to the amino acid sequence |
| | of SEQ ID NO: 10, 17 or 18 of US Pub. No. US |
| | 2010/0278821. |
| CD11a | Raptiva ™ (efalizumab) |
| | Sequence in Werther et al., 1996, The Journal of |
| | Immunology 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID |
| | NO: 124 of US Patent Pub. US 2017/0002060 A1 |
| | VL: the VL sequence of the light chain of |
| | SEQ ID NO: 125 of US Patent Pub. |
| | US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD38 | IB4, HB7 CS/2, clone 90 and NIM-R5 as |
| | disclosed in PCT Pub. WO2015/009726A2 and |
| | references cited therein. |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US |
| | 2017/0002060 A1 |
| | VL: SEQ ID NO: 2 of US Patent Pub. US |
| | 2017/0002060 A1 |
| CD70 | Blenrep ™ (borsetuzumab mafodotin) |
| CD123 | Flotetuzumab |
| CD206 | Anti-CD206 antibodies having a VH a of SEQ ID NO. 2 |
| | and a VL of SEQ ID NO: 4 of WO2003/040169A2 |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID |
| | NOs: 4-6 of U.S. Pat. No. 8,771,690 B2 |
| | CDR-L1, CDR-L2 and CDR-L3 sequences of SEQ ID |
| | NOs: 1-3 of U.S. Pat. No. 8,771,690 B2 |
| CEA | CEA binding portion of RO6958688/RG7802 from |
| | clinical trial NCT02324257 |
| CEA | Cibisatamab |
| CEA | CEA binding portion of MEDI-565/MT110/AMG211 |
| | from clinical trials NCT01284231 and NCT02291614 |
| | VH: SEQ ID NO: 49 or 51 of PCT Publication No. |
| | WO 2013/012414 A1 |
| | VL: SEQ ID NO: 48 of PCT Publication No. WO |
| | 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Clec9a | Anti-Clec9a antibodies having VH and VL amino acids |
| | of SEQ ID NOs. 43 and 48 of PCT Pub. No. |
| | WO2009/026660A1 |
| Clec9a | Anti-Clec9a antibodies having a VH a of SEQ ID NO. 38 |
| | and a VL of SEQ ID NO: 37 or SEQ ID NO: |
| | 43 of PCT Pub. No. WO2022/073062A1 |
| | Anti-Clec9a antibodies having a VH a of SEQ ID NO. 8 |
| | and a VL of SEQ ID NO: 7 of PCT Pub. No. |
| | WO2022/073062A1 |
| Collagen | TRC093 (MT293) |
| alpha-4 | |
| chain | |
| Collagen | The collagen binding antibody fragment described in |
| | Liang et al., 2016, Sci. Rep. 5, 18205; doi: |
| | 10.1038/srep18205 (2016). |
| Collagen | Cetuximab (Erbitux) |
| Type I | |
| Collagen | The amino acid sequences of SEQ ID NO: 1 or 2 of |
| type X | PCT Pub No. WO 2019/020797. |
| Collagen | The amino acid sequences of SEQ ID NO: 1 of PCT |
| type X | Pub No. WO 2014/180992. |
| Collagen | Antibody X34 as described in I. Girkontaite et al., |
| type X | “Immunolocalization of type X collagen in normal fetal |
| | and adult osteoarthritic cartilage with monoclonal |
| | antibodies,” Matrix Biol 15, 231-238 (1996). |
| Collagen | Antibodies X53 or 1H8 or ARC0659 or JF0961 collagen |
| type X | X polyclonal antibody sold under catalog number |
| | PA5-115039 or PA5-116871 or PA5-97603 or |
| | PA5-49198 from ThermoFisher Scientific. |
| Collagen | Antibody sold under catalog number RDI-COLL10abr |
| type X | from RDI. |
| Com- | Soliris ™ (eculizumab) |
| plement | VH: amino acids 1-122 of SEQ ID NO: 10 of U.S. Pat. |
| C5 | No. 6,355,245 |
| | VL: amino acids 3-110 of SEQ ID NO: 9 of U.S. Pat. |
| | No. 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DEC-205 | Anti-DEC-205 antibodies having a VH/VL pair of |
| | SEQ ID NOS. 4/10, 16/22, 28/34, 40/46, 52/58, and |
| | 76/82 of PCT Pub. WO2009/061996A2 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | Mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/ | MDX-447 |
| CD64 | |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of US Patent Pub. No. US |
| | 2020/0317806 A1 |
| | HL: SEQ ID NO: 3 of US Patent Pub. No. US |
| | 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™ (oportuzumab monatox) |
| EpCAM | M701 |
| F protein | Synagic ™ (palivizumab) |
| of RSV | |
| GD2 | 3F8 |
| Glyco- | ReoPro ™ (abiciximab) |
| protein | |
| receptor | |
| IIb/IIIa | |
| gpA33 | MGD007 |
| GPC3 | ERY974 |
| GUCY2C | PF-07062119 |
| Heparanase | An antibody selected from HP130, HP 239, HP 108.264, |
| | HP 115.140, HP 152.197, HP 110.662, HP 144.141, HP |
| | 108.371, HP 135.108, HP 151.316, HP 117.372, HP |
| | 37/33, HP3/17, HP 201 or HP 102 or an amino acid |
| | sequence of SEQ ID NO: 1-11 described in US Patent |
| | Pub. US 2004/0170631. |
| Her2 | Herceptin ™ (trastuzumab) |
| Her2 | Aldesleukin (proleukine) |
| Her2 | Sargramustim (leukine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446, 175. |
| | VL: SEQ ID NO: 2 of U.S. Pat. No. 7,446, 175 |
| IL12/IFN3 | Stelara ™ (ustekinumab) |
| IL1Ra | Antril ™, Kineret ™ (ankinra) |
| IFNR | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 | Actemra ™ (tocilizumab) |
| receptor | VH: SEQ ID NO: 31 of U.S. Pat. No. 7,479,543 |
| | VL: SEQ ID NO: 29 of U.S. Pat. No. 7,479,543 |
| IL12/IFN3 | Stelara ™ (ustekinumab) |
| p40 | VH: SEQ ID NO: 7 of U.S. Pat. No. 6,902,734 |
| subunit | VL: SEQ ID NO: 8 of U.S. Pat. No. 6,902,734 |
| Integrin | Tysabri ™ (natalizumab) |
| α4 | VH: SEQ ID NOs: 11-13 of U.S. Pat. No. 5,840,299 |
| | VL: SEQ ID NOs: 7-8 of U.S. Pat. No. 5,840,299 |
| Integrin | Entyvio ™ (vedolizumab) |
| α4 β7 | HC: SEQ ID NO: 2 of US Patent Pub. US 2012/0282249. |
| | LC: SEQ ID NO: 4 of US Patent Pub. US 2012/0282249. |
| Integrin | VH: SEQ ID NO: 2 of European Patent No. 1 755 659. |
| α5 β1 | VL: SEQ ID NO: 4 of European Patent No. 1 755 659. |
| Integrin | VH: SEQ ID NO: 2, 6, 8, 10, 12, 14, 29-43 or 91-100 |
| β1 | of US Patent Pub. US 2022/0089744. |
| | VL:, SEQ ID NO: 4, 16, 18, 20, 22, 44-57 or 107-116 |
| | of US Patent Pub. US 2022/0089744. |
| KIR | Anti-KIR antibodies having a VH of SEQ ID NO: 5 |
| | and a VL of SEQ ID NO: 3 of PCT Pub. |
| | WO2014/066532A1 |
| KIR | Anti-KIR antibodies having a VH of SEQ ID NO: 1 |
| | and a VL of SEQ ID NO: 2 of PCT Pub. |
| | WO2012/160448A2 Anti-KIR antibodies |
| | having a VH of SEQ ID NO: 3 and a VL of |
| | SEQ ID NO: 4 of PCT Pub. WO2012/160448A2 |
| LAG3 | Relatlimab (BMS-98016) |
| LAG3 | Sym022 |
| LAG3 | HLX26 |
| LAG3 | TSR-033 |
| LAG3 | ABL501 |
| LAG3 | INCAGN02385 |
| LAG3 | Fianlimab (REGN3767) |
| LAG3 | RO7247669 |
| LAG3 | EMB-02 |
| LAG3 | FS118 |
| LAG3 | GSK2831781 |
| LAG3 | IBI323 |
| LAG3 | IBI110 |
| LAG3 | LAG525 |
| LAG3 | XmAb ®22841 |
| LAG3 | LBL-007 |
| LAG3 | VH: SEQ ID NO: 1, 8, 10 or 12 of U.S. Pat. No. |
| | 9,902,772. |
| | VL: SEQ ID NO: 2, 3, 4, 5, 6, 7, 9, 11, 13 or 14 |
| | of U.S. Pat. No. 9,902,772. |
| LAG3 | VH: SEQ ID NO: 182 of U.S. Pat. Pub. |
| | US 2021/0095026. |
| | VL: SEQ ID NO: 88 of U.S. Pat. Pub. |
| | US 2021/0095026. |
| LAG3 | Antibodies having VH/VL amino acid sequences of |
| | SEQ ID NOs 23/24, 3/4 and 11/12 of US Pub. |
| | US2022/0056126A1. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 | Anti-MUC16 antibodies having VH and VL sequences |
| (CA125) | having the amino acid sequences of any one of the |
| | following SEQ ID NO: pairs from US 2018/0118848A1: |
| | 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274, 160. |
| | VL: SEQ ID NO: 2 of U.S. Pat. No. 11,274, 160. |
| NGF | (tanezumab) |
| NKH1A | The monoclonal antibody deposited with ATCC and |
| | assigned accession no. HB8564, as described in U.S. |
| | Pat. No. 4,772,552A |
| NKP46 | Anti-NKP46 antibodies having CDR-H1, CDR-H2 and |
| | CDR-H3 sequences of SEQ ID NOs: 4, 6 and 8 and |
| | CDR-L1, CDR-L2 and CDR-L3 sequences of SEQ ID |
| | NOs: 12, 14 and 16 of PCT Pub. WO2018/047154A1 |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106/BMS-936558 (nivolumab), a human IgG4 |
| | mAb with the structure described in WHO Drug |
| | Information, Vol. 27, No. 1, pages 68-69 (2013) and |
| | whose heavy and light chain sequences are disclosed in |
| | FIG. 7 of US Pub. No. US20190270812A1 |
| PD1 | MK-3475 (pembrolizumab), a humanized IgG4 mAb |
| | with the structure described in WHO Drug Information, |
| | Vol. 27, No. 2, pages 161-162 (2013) and whose heavy |
| | and light chain sequences are disclosed in FIG. 6 of |
| | US Pub. No. US20190270812A1 |
| PD1 | REGN2810 (disclosed as H4H7798N in US Pub No. |
| | 20150203579) |
| PD1 | Anti-PD1 antibodies having CDR H1-H3 and CDR |
| | L1-L3 sequences corresponding to the following |
| | SEQ ID NOs. of U.S. Pat. No. 11,034,765 B2: |
| | a) SEQ ID NOs: 18, 19, 20, 21, 22, and 23, respectively; |
| | b) SEQ ID NOs: 24, 25, 26, 27, 28, and 29, respectively; |
| | c) SEQ ID NOs: 30, 31, 32, 33, 34, and 35, respectively; |
| | d) SEQ ID NOs: 36, 37, 38, 39, 40, and 41, respectively; |
| | e) SEQ ID NOs: 42, 43, 44, 45, 46, and 47, respectively; |
| | f) SEQ ID NOs: 48, 49, 50, 51, 52, and 53, respectively; |
| | g) SEQ ID NOs: 54, 55, 56, 57, 58, and 59, respectively; |
| | and |
| | h) SEQ ID NOs: 60, 61, 62, 63, 64, and 65, respectively. |
| PD1 | Anti-PD1 antibodies disclosed in Tables 1-3 of PCT |
| | Pub. WO2015112800A1, including but not limited to |
| | anti-PD1 antibodies having VH/VL pairs having SEQ |
| | ID NOs: 2/10, 18/26, 34/42, 50/58, 66/74, 82/90, |
| | 98/106, 1 14/122, 130/138, 146/154, 162/170, 178/186, |
| | 194/202, 210/202, 218/202, 226/202, 234/202, 242/202, |
| | 250/202, 258/202, 266/202, 274/202, 282/202, 290/202, |
| | 298/186, 306/186 and 314/186 of PCT Pub. |
| | WO2015112800A1. |
| PD1 | Anti-PD1 antibodies disclosed in U.S. Pat. No. |
| | 10,294,299 B2 as having the following SEQ ID NO. |
| | pair for heavy and light chain variable domains: |
| | SEQ ID NOs. 164/178 |
| | SEQ ID NOs. 165/179 |
| | SEQ ID NOs. 166/180 |
| | SEQ ID NOs. 167/181 |
| | SEQ ID NOs. 168/182 |
| | SEQ ID NOs. 169/183 |
| | SEQ ID NOs. 170/184 |
| | SEQ ID NOs. 171/185 |
| | SEQ ID NOs. 172/186 |
| | SEQ ID NOs. 173/187 |
| | SEQ ID NOs. 174/188 |
| | SEQ ID NOs. 175/189 |
| | SEQ ID NOs. 176/190 |
| | SEQ ID NOs. 177/190 |
| PD1 | MEDI-0680 (AMP-514) |
| PD1 | PDR001 |
| PD1 | BGB-108 |
| PD1 | h409A11, described in WO2008/156712 |
| PD1 | h409A16, described in WO2008/156712 |
| PD1 | h409A17, described in WO2008/156712 |
| PD1 | Anti-PD1 antibodies described in U.S. Pat. No. 7488802 |
| PD1 | Anti-PD1 antibodies described in U.S. Pat. No. 7521051 |
| PD1 | Anti-PD1 antibodies described in U.S. Pat. No. 8008449 |
| PD1 | Anti-PD1 antibodies described in U.S. Pat. No. 8354509 |
| PD1 | Anti-PD1 antibodies described in U.S. Pat. No. 8168757 |
| PD1 | Anti-PD1 antibodies described in PCT Pub. No. |
| | W02004/004771 |
| PD1 | Anti-PD1 antibodies described in PCT Pub. No. |
| | W02004/056875 |
| PD1 | Anti-PD1 antibodies described in PCT Pub. No. |
| | W02004/072286 |
| PD1 | Anti-PD1 antibodies described in US Pub. No. |
| | US2011/0271358 |
| PDL1 | Durvalumab (MEDI4736) |
| PDL1 | Atezolizumab (Tecentriq, MPDL3280A)) |
| PDL1 | MDX 1105 (BMS-936559) |
| PDL1 | Avelumab |
| PDL1 | ZKAB001 (Socazolimab) |
| PDL1 | TQB2450 |
| PDL1 | MEDI4736 |
| PDL1 | HLX20 |
| PDL1 | KN035 |
| PDL1 | LY3434172 |
| PDL1 | LY3300054 |
| PDL1 | LDP |
| PDL1 | EMB-09 |
| PDL1 | ABL501 |
| PDL1 | INBRX-105 |
| PDL1 | SHR-1210 |
| PDL1 | STI-3031 (IMC-001) |
| PDL1 | MPDL3280A (RG7446) |
| PDL1 | KN035 |
| PDL1 | BGB-A333 |
| PDL1 | HLX301 |
| PDL1 | Y101D |
| PDL1 | ES101 |
| PDL1 | IBI322 |
| PDL1 | Envafolimab |
| PDL1 | VH: SEQ ID NO: 46, 48, 50 or 52 of U.S. Pat. No. |
| | 11,168,144. |
| | VL: SEQ ID NO: 58, 137 or 12 of U.S. Pat. No. 11, |
| | 168,144. |
| PDL1 | VH: SEQ ID NO: 23, 124, 126, 127, 128, 130, 140 or |
| | 145 of U.S. Pat. No. 11,208,486. |
| | VL: SEQ ID NO: 24 or 125 of U.S. Pat. No. 11,208,486. |
| Phosphati- | (bavituximab) |
| dylserine | |
| PSCA | GEM3PSCA |
| PSMA | huJ591 |
| PSMA | Anti-PSMA antibodies having VH and VL sequences |
| | having the amino acid sequences of any one of the |
| | following SEQ ID NO: pairs from WO 2017/023761A1: |
| | 2/1642; 10/1642; 18/1642; 26/1642; 34/1642; 42/1642; |
| | 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, |
| | PSMA 3.11, PSMA 5.4, PSMA 7.1, PSMA 7.3, PSMA |
| | 10.3, PSMA 1.8.3, PSMA A3.1.3, PSMA A3.3.1, |
| | Abgenix 4.248.2, Abgenix 4.360.3, Abgenix 4.7.1, |
| | Abgenix 4.4.1, Abgenix 4.177.3, Abgenix 4.16.1, |
| | Abgenix 4.22.3, Abgenix 4.28.3, Abgenix 4.40.2, |
| | Abgenix 4.48.3, Abgenix 4.49.1, Abgenix 4.209.3, |
| | Abgemx 4.219.3, Abgenix 4.288.1, Abgenix 4.333.1, |
| | Abgemx 4.54.1, Abgenix 4.153.1, Abgenix 4.232.3, |
| | Abgenix 4.292.3, Abgenix 4.304.1, Abgenix 4.78.1 |
| | and Abgenix 4.152.1 described in WO2003034903A2 |
| | A hybridoma cell line such as: PSMA 3.7 (PTA-3257), |
| | PSMA 3.8, PSMA 3.9 (PTA-3258), PSMA 3.11 (PTA- |
| | 3269), PSMA 5.4 (PTA-3268), PSMA 7.1 (PTA-3292), |
| | PSMA 7.3 (PTA-3293), PSMA 10.3 (PTA-3247), |
| | PSMA 1.8.3 (PTA-3906), PSMA A3.1.3 (PTA-3904), |
| | PSMA A3.3.1 (PTA-3905), Abgenix 4.248.2 |
| | (PTA-4427), Abgenix 4.360.3 (PTA-4428), Abgenix |
| | 4.7.1 (PTA-4429), Abgenix 4.4.1 (PTA-4556), Abgenix |
| | 4.177.3 (PTA-4557), Abgenix 4.16.1 (PTA-4357), |
| | Abgenix 4.22.3 (PTA-4358), Abgenix 4.28.3 |
| | (PTA-4359), Abgenix 4.40.2 (PTA-4360), Abgenix |
| | 4.48.3 (PTA-4361), Abgenix 4.49.1 (PTA-4362), |
| | Abgenix 4.209.3 (PTA-4365), Abgenix 4.219.3 |
| | (PTA-4366), Abgenix 4.288.1 (PTA-4367), Abgenix |
| | 4.333.1 (PTA-4368), Abgenix 4.54.1 (PTA-4363), |
| | Abgenix 4.153.1 (PTA-4388), Abgenix 4.232.3 |
| | (PTA-4389), Abgenix 4.292.3 (PTA-4390), Abgenix |
| | 4.304.1 (PTA-4391), Abgenix 4.78.1 (PTA-4652), |
| | and Abgemx 4.152.1(PTA-4653) described in |
| | WO 2003/034903A2. |
| | VH of SEQ ID NOs: 2-7 described in WO 2003/ |
| | 034903A2 |
| | VL of SEQ ID NOs: 8-13 described in WO 2003/ |
| | 034903A2 |
| PMSA | VH: SEQ ID NOs: 225, 239, 253, 267, 281, 295, 309, |
| | 323, 337, 351, 365, 379, 393, 407, 421, 435, 449, 463, |
| | 477, 491, 505, 519, 533, 547, 561, 575, 589, 603 or 617 |
| | described in WO 2011/121110A1. |
| | VL SEQ ID NOs: 230, 244, 258, 272, 286, 300, 314, |
| | 328, 342, 356, 370, 384, 398, 412, 426, 440, 454, 468, |
| | 482, 496, 510, 524, 538, 552, 566, 580, 594, 608 or |
| | 622 described in WO 2011/121110A1. |
| | VH and VL SEQ ID Nos: 235, 249, 263, 277, 291, 305, |
| | 319, 333, 347, 361, 375, 389, 403, 417, 431, 445, 459, |
| | 473, 487, 501, 515, 529, 543, 557, 571, 585, 599, 613 |
| | or 627 described in WO 2011/121110A1. |
| PMSA | An anti-PMSA antibody having a VL amino acid |
| | sequence of any one of SEQ ID NOs: 229-312 |
| | of US 2022/0119525 A1 and a VH of |
| | SEQ ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of U.S. Patent Pub. 2017/0002060 |
| | VL: SEQ ID NO: 52 of U.S. Patent Pub. 2017/0002060 |
| SLAMF7 | Empliciti ™ (elotuzumab) |
| SSTR2 | XmAb ®18087 |
| STEAP1 | VHCDR1 SEQ ID NOs: 14, 33, 182, 184 or 185 |
| | described in US20210179731A1. |
| | VHCDR2 SEQ ID NOs: 15, 21, 34, 182, 184 or 185 |
| | described in US20210179731A1. |
| | VHCDR3 SEQ ID NOs: 16 and 35 described in |
| | US20210179731A1. |
| | VH SEQ ID NOs: 182 or 184 described in |
| | US20210179731A1. |
| | VLCDR1 SEQ ID NOs: 11 or 30 described in |
| | US20210179731A1. |
| | VLCDR2 SEQ ID NOs: 12 or 31 described in |
| | US20210179731A1. |
| | VLCDR3 SEQ ID NOs: 13 or 32 described in |
| | US20210179731A1. |
| | VL SEQ ID NOs: 183 or 186 described in |
| | US20210179731A1. |
| STEAP1 | AMG509 |
| STEAP2 | Anti-STEAP 2 antibodies having CDR-H1, CDR-H2, |
| | CDR-H3, CDR-L1, CDR-L2 and CDR-L3 sequences |
| | selected from SEQ ID NOS: (1) 4-6-8-12-14-16; |
| | (2) 20-22-24-28-30-32; (3) 36-38-40-44-46-48; |
| | (4) 52-54-56-60-62-64; (5) 68-70-72-60-62-64; |
| | (6) 76-78-80-60-62-64; (7) 84-86-88-60-62-64; |
| | (8) 92-94-96-60-62-64; (9) 100-102-104-60-62- |
| | 64; (10) 108-110-112-116-118-120; (11) 124- |
| | 126-128-132-134-136; (12) 140-142-144-148- |
| | 150-152; (13) 156-158-160-164-166-168; (14) |
| | 172-174-176-180-182-184; (15) 188-190-192- |
| | 196-198-200; (16) 204-206-208-212-214-216; |
| | (17) 220-222-224-228-230-232; (18) 236-238- |
| | 240-244-246-248; (19) 252-254-256-260-262- |
| | 264; (20) 268-270-272-276-278-280; (21) 284- |
| | 286-288-292-294-296; (22) 300-302-304-308- |
| | 310-312; (23) 316-318-320-324-326-328; (24) |
| | 332-334-336-340-342-344; (25) 348-350-352- |
| | 356-358-360; (26) 364-366-368-372-374-376; |
| | and (27) 380-382-384-388-390-392 of U.S. |
| | Pat. No. 10,772,972 B2. |
| | Anti-STEAP 2 antibodies having (a) a VH |
| | comprising the amino acid of any one of SEQ |
| | ID NOs: 2, 18, 34, 50, 66, 74, 82, 90, 98, 106, 122, |
| | 138, 154, 170, 186, 202, 218, 234, 250, 266, 282, |
| | 298, 314, 330, 346, 362, and 378 of U.S. Pat. No. |
| | 10,772,972 B2; and (b) a VL comprising the |
| | amino acid sequence of any one of SEQ ID NOs: |
| | 10; 26; 42; 58; 114; 130; 146; 162; 178; 194; 210; |
| | 226, 242; 258; 274; 290; 306; 322; 338; 354; 370; |
| | and 386 of U.S. Pat. No. 10,772,972 B2. |
| | Anti-STEAP 2 antibodies having a VH/VL pair |
| | comprising the amino acid sequences of any |
| | of the following pairs of SEQ ID NOs of U.S. Pat. |
| | No. 10,772,972 B2: 2/10; 18/26; 34/42; 50/58; |
| | 66/58; 74/58; 82/58; 90/58; 98/58; 106/114; |
| | 122/130; 138/146; 154/162; 170/178; 186/194; |
| | 202/210; 218/226; 234/242; 250/258; 266/274; |
| | 282/290; 298/306; 314/322; 330/338; 346/354; |
| | 362/370; and 378/386. |
| Syndecan-1 | The B-B4 antibody described in Wijdenes et al. |
| (CD 138) | (1996) Br. J. Haematol., 94: 318-323 |
| Syndecan-4 | The amino acid sequence of amino acids 93 and |
| | 121 of SEQ ID NO: 1 or the amino acid sequence of |
| | amino acids 92 and 122 of SEQ ID NO: 2 described |
| | in European Patent Pub. EP 2 603 236. |
| TGFβ | GC1008 |
| TNFR | Enbrel ™ (etanercept) |
| TNFα | Remicade ™ (infliximab) |
| | VH: SEQ ID NO: 2 of Int. Patent Publication |
| | WO201/3087911 A1 |
| | VH: SEQ ID NO: 3 of Int. Patent Publication |
| | WO2013/A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012, 135 |
| TNFα | Simponi ™ (golimumab) |
| | VH: SEQ ID NO: 7 of U.S. Pat. No. 7,250, 165 |
| | VL: SEQ ID NO: 8 of U.S. Pat. No. 7,250, 165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |
| XCR1 | Anti-XCR1 antibodies disclosed in U.S. Pat. No. |
| | 9,371,389 B2, including: |
| | The antibodies designated 2H6, 5G7, 11H2, |
| | HK1L2 and HK5L5 |
| | Antibodies having CDR-H1, CDR-H2 and CDR-H3 |
| | sequences of SEQ ID NOs: 53-55 and CDR-L1, |
| | CDR-L2 and CDR-L3 sequences of SEQ ID |
| | NOs: 56-58. |
| | Antibodies having CDR-H1, CDR-H2 and CDR-H3 |
| | sequences of SEQ ID NOs: 41-43 and CDR-L1, |
| | CDR-L2 and CDR-L3 sequences of SEQ ID |
| | NOs: 44-46. |

In some aspects, the targeting moiety competes with an antibody set forth in Table F for binding to the target molecule. In further aspects, the targeting moiety comprises CDRs having CDR sequences of an antibody set forth in Table F. In some embodiments, the targeting moiety comprises all 6 CDR sequences of the antibody set forth in Table F. In other embodiments, the targeting moiety comprises at least the heavy chain CDR sequences (CDR-H1, CDR-H2, CDR-H3 and the light chain CDR sequences of a universal light chain. In further aspects, a targeting moiety comprises a VH comprising the amino acid sequence of the VH of an antibody set forth in Table F. In some embodiments, the targeting moiety further comprises a VL comprising the amino acid sequence of the VL of the antibody set forth in Table F. In other embodiments, the targeting moiety further comprises a universal light chain VL sequence.

In some embodiments, the targeting moieties target PDL1, as set forth in Table F-1 below, together with references to exemplary antibodies or antibody sequences upon which the targeting moiety can be based.

TABLE F-1
Exemplary PDL1 antibodies and/or binding sequences

| Target | Antibody Name and/or Binding Sequences |
|---|---|
| PDL1 | Durvalumab (MEDI4736) |
| | HC: SEQ ID NO: 26 of PCT application No. WO2020225552 |
| | LC: SEQ ID NO: 27 of PCT application No. WO2020225552 |
| PDL1 | Atezolizumab (Tecentriq, MPDL3280A, RG7446) |
| | HC: SEQ ID NO: 20 of U.S. Pat. No. 8,217,149 |
| | LC: SEQ ID NO: 21 of U.S. Pat. No. 8,217,149 |
| PDL1 | MDX 1105 (BMS-936559) |
| PDL1 | Anti-PDL1 antibodies described in U.S. Pat. No. 7,943,743 as having the |
| | following SEQ ID NO. pairs for heavy and light chain variable domains: |
| | SEQ ID Nos: 1/11 |
| | SEQ ID Nos: 2/12 |
| | SEQ ID Nos: 3/13 |
| | SEQ ID Nos: 4/14 |
| | SEQ ID Nos: 5/15 |
| | SEQ ID Nos: 6/16 |
| | SEQ ID Nos: 7/17 |
| | SEQ ID Nos: 8/18 |
| | SEQ ID Nos: 9/19 |
| | SEQ ID Nos: 10/20 |
| PDL1 | Avelumab, described in U.S. Pat. No.: 9,624,298 as having |
| | the following heavy and light chain variable domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID Nos: 52, 56, and 77 of |
| | PCT Pub. No. 2018/080812 |
| | CDR-L1, CDR-L2 and CDR-L3 sequences of SEQ ID Nos: 65, 42, and 71 of |
| | PCT Pub. No. 2018/080812 |
| PDL1 | KN035 (Envafolimab) is a nanobody described as Hu56V2 in U.S. Pat. No. |
| | 11,225,522 as having the VHH SEQ ID NO: 34 |
| PDL1 | LY3434172 |
| PDL1 | LY3300054 (lodapolimab) described in PCT Pub No: WO 2017/034916 as |
| | having the following heavy and light chain variable domains: |
| | HC: SEQ ID NO: 10 |
| | L: SEQ ID NO: 11 |
| PDL1 | LDP (lesabelimab, ADG104) described in CN Patent No: 114225023 as |
| | having the following heavy and light chain variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No. 10,118,963 as having the |
| | following heavy and light chain variable domains: |
| | HC: SEQ ID NO: 1 |
| | LC: SEQ ID NO: 2 |
| PDL1 | BGB-A333 (garivulimab) described in U.S. Pat. No. 11,512,132 as having the |
| | following heavy and light chain variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or 145 of U.S. Pat. No. |
| | 11,208,486. |
| | VL: SEQ ID NO: 24 or 125 of U.S. Pat. No. 11,208,486. |

In some aspects, the targeting moiety competes with an antibody set forth in Table F-1 for binding to the target molecule. In further aspects, the targeting moiety comprises CDRs having CDR sequences of an antibody set forth in Table F-1. In some embodiments, the targeting moiety comprises all 6 CDR sequences of the antibody set forth in Table F. In other embodiments, the targeting moiety comprises at least the heavy chain CDR sequences (CDR-H1, CDR-H2, CDR-H3 and the light chain CDR sequences of a universal light chain. In further aspects, a targeting moiety comprises a VH comprising the amino acid sequence of the VH of an antibody set forth in Table F-1. In some embodiments, the targeting moiety further comprises a VL comprising the amino acid sequence of the VL of the antibody set forth in Table F-1. In other embodiments, the targeting moiety further comprises a universal light chain VL sequence.

In some embodiments, the targeting moieties target PD1, as set forth in Table F-2 below, together with references to exemplary antibodies or antibody sequences upon which the targeting moiety can be based.

TABLE F-2
Exemplary PD1 antibodies and/or binding sequences

| Target | Antibody Name and/or Binding Sequences |
|---|---|
| PD1 | MDX-1106/BMS-936558 (nivolumab), a human IgG4 mAb with the structure |
| | described in WHO Drug Information, Vol. 27, No. 1, pages 68-69 (2013) and |
| | whose heavy and light chain sequences are disclosed in FIG. 7 of U.S. Pub. No. |
| | US20190270812A1 |
| | HC: SEQ ID NO: 23 of U.S. Pub. No. US20190270812A1 |
| | LC: SEQ ID NO: 24 of U.S. Pub. No. US20190270812A1 |
| PD1 | MK-3475 (pembrolizumab), a humanized IgG4 mAb with the structure described |
| | in WHO Drug Information, Vol. 27, No. 2, pages 161-162 (2013) and whose |
| | heavy and light chain sequences are disclosed in FIG. 6 of U.S. Pub. No. |
| | US20190270812A1 |
| | HC: SEQ ID NO: 21 of U.S. Pub. No. US20190270812A1 |
| | LC: SEQ ID NO: 22 of U.S. Pub. No. US20190270812A1 |
| PD1 | H4H7798N as disclosed in U.S. Pub No. 20150203579 |
| | HC: SEQ ID NO: 330 of U.S. Pub. No. 20150203579 |
| | LC: SEQ ID NO: 331 of U.S. Pub. No. 20150203579 |
| PD1 | Anti-PD1 antibodies having CDR H1-H3 and CDR L1-L3 sequences |
| | corresponding to the following SEQ ID Nos. of U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; and |
| | h) SEQ ID Nos: 60, 61, 62, 63, 64, and 65, respectively. |
| PD1 | Anti-PD1 antibodies disclosed in Tables 1-3 of PCT Pub. WO2015112800A1, |
| | including but not limited to anti-PD1 antibodies having VH/VL pairs having SEQ |
| | ID Nos: 2/10, 18/26, 34/42, 50/58, 66/74, 82/90, 98/106, 1 14/122, 130/138, |
| | 146/154, 162/170, 178/186, 194/202, 210/202, 218/202, 226/202, 234/202, |
| | 242/202, 250/202, 258/202, 266/202, 274/202, 282/202, 290/202, 298/186, |
| | 306/186 and 314/186 of PCT Pub. WO2015112800A1. |
| PD1 | Anti-PD1 antibodies disclosed in U.S. Pat. No. 10,294,299 B2 as having the |
| | following SEQ ID NO. pairs for heavy and light chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb whose heavy and light chain |
| | sequences are disclosed as BAP049-Clone-E in U.S. Pat. No.: 9683048 B2. |
| | HC: SEQ ID NO: 91 of U.S. Pat. No.: 9,683,048 |
| | LC: SEQ ID NO: 72 of U.S. Pat. No.: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. 7,488,802 as having the |
| | following SEQ ID NO. pairs for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 7,521,051 |
| | as having the following SEQ ID NO. pairs for heavy and light chain variable |
| | domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 8,008,449 |
| | as having the following SEQ ID NO. pairs for heavy and light chain variable |
| | domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. 8,354,509 as having the |
| | following SEQ ID NO. pairs for heavy and light chain full-length domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. 8,168,757 as having the |
| | following SEQ ID NO. pairs for heavy and light chain variable domains: |
| | SEQ ID Nos. 4/5 |
| | SEQ ID Nos. 12/13 |
| | SEQ ID Nos. 18/19 |
| | SEQ ID Nos. 40/41 |
| | SEQ ID Nos. 47/48 |
| | SEQ ID Nos.26/27 |
| | SEQ ID Nos.34/35 |
| | SEQ ID Nos.55/56 |
| | SEQ ID Nos.67/68 |
| PD1 | Anti-PD1 antibodies described in PCT Pub. No. WO2004/004771 |
| PD1 | Anti-PD1 antibodies described in PCT Pub. No. WO2004/056875 as having the |
| | following SEQ ID NO. pairs for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. WO2004/072286 |
| PD1 | VH: SEQ ID NO: 25, 26, 27, 28, or 29 of U.S. Pub. No. US2011/0271358 |
| | VL: SEQ ID NO: 30, 31, 32, or 33 of U.S. Pub. No. US2011/0271358 |
| PD1 | SHR-1210 (Camrelizumab) described in PCT Publication No: WO |
| | 2015/085847 as having the following heavy and light chain variable domains: |
| | HC: SEQ ID NO: 9 |
| | LC: SEQ ID NO: 10 |

In some aspects, the targeting moiety competes with an antibody set fort in Table F-2 for binding to the target molecule. In further aspects, the targeting moiety comprises CDRs having CDR sequences of an antibody set forth in Table F-2. In some embodiments, the targeting moiety comprises all 6 CDR sequences of the antibody set forth in Table F. In other embodiments, the targeting moiety comprises at least the heavy chain CDR sequences (CDR-H1, CDR-H2, CDR-H3 and the light chain CDR sequences of a universal light chain. In further aspects, a targeting moiety comprises a VH comprising the amino acid sequence of the VH of an antibody set forth in Table F-2. In some embodiments, the targeting moiety further comprises a VL comprising the amino acid sequence of the VL of the antibody set forth in Table F-2. In other embodiments, the targeting moiety further comprises a universal light chain VL sequence.

Additional target molecules that can be targeted by the IFN receptor antagonists are disclosed in Table I below and in, e.g., Hafeez et al., 2020, Molecules 25:4764, doi:10.3390/molecules25204764, particularly in Table 1. Table 1 of Hafeez et al. is incorporated by reference in its entirety here.

### 6.6.1.1. Targeting Moiety Formats

In certain aspects, the targeting moiety of an IFN receptor antagonist of the disclosure can be any type of antibody or fragment thereof that retains specific binding to an antigenic determinant. In one embodiment the targeting moiety is an immunoglobulin molecule or fragment thereof, particularly an IgG class immunoglobulin molecule, more particularly an IgG₁ or IgG₄ immunoglobulin molecule. Antibody fragments include, but are not limited to, VH (or V_{H}) fragments, VL (or V_{L}) fragments, Fab fragments, F(ab′)₂ fragments, scFv fragments, Fv fragments, minibodies, diabodies, triabodies, and tetrabodies.

### 6.6.1.1.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

For the IFN receptor antagonists of the disclosure, particularly when the light chains of the targeting moieties are not common or universal light chains, it is advantageous to use Fab heterodimerization strategies to permit the correct association of Fab domains belonging to the same targeting moiety and minimize aberrant pairing of Fab domains belonging to different targeting moieties. For example, the Fab heterodimerization strategies shown in Table G below can be used:

TABLE G
Fab Heterodimerization Strategies

| STRATEGY | VH | CH1 | VL | CL | REFERENCE |
|---|---|---|---|---|---|
| CrossMabC | WT | CL domain | WT | CH1 domain | Schaefer et al., |
| H1-CL | | | | | 2011, Cancer Cell |
| | | | | | 2011; 20:472-86; |
| | | | | | PMID:22014573. |
| orthogonal | 39K, 62E | H172A, | 1R, 38D, | L135Y, | Lewis et al., 2014, |
| Fab | | F174G | (36F) | S176W | Nat Biotechnol |
| VHVRD1CH | | | | | 32:191-8 |
| 1CRD2- | | | | | |
| VLVRD1Cλ | | | | | |
| CRD2 | | | | | |
| orthogonal | 39Y | WT | 38R | WT | Lewis et al., 2014, |
| Fab | | | | | Nat Biotechnol |
| VHVRD2CH | | | | | 32:191-8 |
| 1 wt- | | | | | |
| VLVRD2Cλ | | | | | |
| wt | | | | | |
| TCR CaCβ | 39K | TCR Cα | 38D | TCR Cβ | Wu et al., 2015, |
| | | | | | MAbs 7:364-76 |
| CR3 | WT | T192E | WT | N137K, | Golay at al., 2016, J |
| | | | | S114A | Immunol 196:3199- |
| | | | | | 211. |
| MUT4 | WT | L143Q, | WT | V133T, | Golay at al., 2016, J |
| | | S188V | | S176V | Immunol 196:3199- |
| | | | | | 211. |
| DuetMab | WT | F126C | WT | S121C | Mazor et al., 2015, |
| | | | | | MAbs 7:377-89; |
| | | | | | Mazor et al., 2015, |
| | | | | | MAbs 7:461-669. |
| Domain | WT | CH3 + knob | WT | CH3 + hole | Wozniak-Knopp et |
| exchanged | | or hole | | or knob | al., 2018, |
| | | mutation | | mutation | PLOSONE13(4):e01 |
| | | | | | 95442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers in Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each unique ABD in the IFN receptor antagonists of the disclosure. In various embodiments, employing a common light chain as described herein reduces the number of inappropriate species in the IFN receptor antagonists as compared to employing original cognate VLs. In various embodiments, the VL domains of ABDs are identified from monospecific antibodies comprising a common light chain. In various embodiments, the VH regions of the ABDs in the IFN receptor antagonists comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39Jκ5 sequence or a rearranged human Vκ3-20Jκ1 sequence, and include somatically mutated (e.g., affinity matured) versions. See, for example, U.S. Pat. No. 10,412,940.

6.6.1.1.2. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5 (typically a repeat of a sequence containing the amino acids glycine and serine, such as the amino acid sequence (Gly4-Ser)3 (SEQ ID NO:50), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.7. Separator Moiety

The IFN receptor antagonists of the disclosure include one or more separator moieties. As a component of an IFN receptor antagonist, a separator moiety may be any molecule that enables simultaneous binding of the anchoring moiety and the IFN moiety. Accordingly, a separator moiety is of particular characteristics (e.g., size, shape, steric properties, etc.) so as to provide sufficient separation between the anchoring moiety and the IFN moiety for each to bind to their respective targets. A separator moiety may be, for example, an organic polymer or a polypeptide.

A separator moiety may, in addition to enabling simultaneous binding of the anchoring moiety and the IFN moiety, further have one or more additional properties. For example, in certain embodiments, a separator moiety is a multimerization moiety. A “multimerization moiety” describes any polypeptide or other molecule or portion thereof capable of multimerization (e.g., dimerization). Such multimerization includes, for example, non-covalent association of two or more multimerization moieties. Various multimerization moieties are recognized in the art and contemplated herein.

Exemplary separator moieties of the present disclosure are described further below.

### 6.7.1. Fc Domains and Fc Regions

The IFN receptor antagonists of the disclosure typically include a pair of Fc domains that associate to form an Fc region. In native antibodies, Fc regions comprise hinge regions at their N-termini to form a constant domain. Throughout this disclosure, the reference to an Fc domain encompasses an Fc domain with a hinge domain at its N-terminus unless specified otherwise.

The Fc domains can be derived from any suitable species operably linked to an ABD or component thereof. In one embodiment the Fc domain is derived from a human Fc domain. In preferred embodiments, the targeting moiety or component thereof is fused to an IgG Fc molecule. A targeting moiety or component thereof may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific binding molecules, e.g., the IFN receptor antagonists of the disclosure and MBMs produced by their activation, the Fc domains might advantageously be different to allow for heterodimerization, as described in Section 6.7.1.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In IFN receptor antagonists of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the IFN receptor antagonists of the present disclosure may include variants of the naturally occurring constant domains described above. Such variants may comprise one or more amino acid variations compared to wild type constant domains. In one example the Fc region of the present disclosure comprises at least one constant domain that varies in sequence from the wildtype constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the IFN receptor antagonists of the present disclosure do not comprise a tailpiece.

The Fc domains that are incorporated into the IFN receptor antagonists of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.7.1.1.

The Fc domains can also be altered to include modifications that improve manufacturability of asymmetric IFN receptor antagonists, for example by allowing heterodimerization, which is the preferential pairing of non-identical Fc domains over identical Fc domains. Heterodimerization permits the production of IFN receptor antagonists in which different polypeptide components are connected to one another by an Fc region containing Fc domains that differ in sequence. Examples of heterodimerization strategies are exemplified in Section 6.7.1.2.

It will be appreciated that any of the modifications mentioned above can be combined in any suitable manner to achieve the desired functional properties and/or combined with other modifications to alter the properties of the IFN receptor antagonists.

Example Fc domain sequences are provided in Table C, below.

TABLE C
Fc Sequences

| | | SEQ |
|---|---|---|
| Fc | Sequence | ID NO |
| hIgG1 Fc | EPKSCDKTHTCPPCPAPELLGGPSVFLFPPKPKDTLMISRTPEVTCVVVDVS | 140 |
| (amino acids 99- | HEDPEVKFNWYVDGVEVHNAKTKPREEQYNSTYRVVSVLTVLHQDWLNGKEY | |
| 330 of UniprotKB | KCKVSNKALPAPIEKTISKAKGQPREPQVYTLPPSRDELTKNQVSLTCLVKG | |
| P01857-1) | FYPSDIAVEWESNGQPENNYKTTPPVLDSDGSFFLYSKLTVDKSRWQQGNVF | |
| | SCSVMHEALHNHYTQKSLSLSPGK | |
| hIgG2 Fc | ERKCCVECPPCPAPPVAGPSVFLFPPKPKDTLMISRTPEVTCVVVDVSHEDP | 141 |
| (amino acids 99- | EVQFNWYVDGVEVHNAKTKPREEQFNSTFRVVSVLTVVHQDWLNGKEYKCKV | |
| 326 of UniprotKB | SNKGLPAPIEKTISKTKGQPREPQVYTLPPSREEMTKNQVSLTCLVKGFYPS | |
| P01859-1) | DISVEWESNGQPENNYKTTPPMLDSDGSFFLYSKLTVDKSRWQQGNVFSCSV | |
| | MHEALHNHYTQKSLSLSPGK | |
| hIgG3 Fc | ELKTPLGDTTHTCPRCPEPKSCDTPPPCPRCPEPKSCDTPPPCPRCPEPKSC | 142 |
| (amino acids 99- | DTPPPCPRCPAPELLGGPSVFLFPPKPKDTLMISRTPEVTCVVVDVSHEDPE | |
| 377 of UniprotKB | VQFKWYVDGVEVHNAKTKPREEQYNSTFRVVSVLTVLHQDWLNGKEYKCKVS | |
| P01860-1) | NKALPAPIEKTISKTKGQPREPQVYTLPPSREEMTKNQVSLTCLVKGFYPSD | |
| | IAVEWESSGQPENNYNTTPPMLDSDGSFFLYSKLTVDKSRWQQGNIFSCSVM | |
| | HEALHNRFTQKSLSLSPGK | |
| hIgG4 Fc | ESKYGPPCPSCPAPEFLGGPSVFLFPPKPKDTLMISRTPEVTCVVVDVSQED | 143 |
| (amino acids 99- | PEVQFNWYVDGVEVHNAKTKPREEQFNSTYRVVSVLTVLHQDWLNGKEYKCK | |
| 327 of UniprotKB | VSNKGLPSSIEKTISKAKGQPREPQVYTLPPSQEEMTKNQVSLTCLVKGFYP | |
| P01861-1) | SDIAVEWESNGQPENNYKTTPPVLDSDGSFFLYSRLTVDKSRWQEGNVFSCS | |
| | VMHEALHNHYTQKSLSLSLGK | |
| hIgG4s Fc | ESKYGPPCPPCPAPPVAGPSVFLFPPKPKDTLMISRTPEVTCVVVDVSQEDP | 144 |
| | EVQFNWYVDGVEVHNAKTKPREEQFNSTYRVVSVLTVLHQDWLNGKEYKCKV | |
| | SNKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCSV | |
| | MHEALHNHYTQKSLSLSLGK | |
| hIgG1 PVA Fc | EPKSCDKTHTCPPCPAPPVAGPSVFLFPPKPKDTLMISRTPEVTCVVVDVSH | 145 |
| | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 LALAPG Fc | EPKSCDKTHTCPPCPAPEAAGGPSVFLFPPKPKDTLMISRTPEVTCVVVDVS | 146 |
| | HEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| hIgG1 PVA star | EPKSCDKTHTCPPCPAPPVAGPSVFLFPPKPKDTLMISRTPEVTCVVVDVSH | 147 |
| | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| hIgG1s | DKKVEPKSCDKTHTCPPCPAPPVAGPSVFLFPPKPKDTLMISRTPEVTCVVV | 148 |
| | DVSQEDPEVQFNWYVDGVEVHNAKTKPREEQFNSTYRVVSVLTVLHQDWING | |
| | KEYKCKVSNKGLPSSIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |
| hIgG1 N180G, | EPKSCDKTHTCPPCPAPELLGGPSVFLFPPKPKDTLMISRTPEVTCVVVDVS | 149 |
| also referred | HEDPEVKFNWYVDGVEVHNAKTKPREEQYGSTYRVVSVLTVLHQDWLNGKEY | |
| to as N297G | KCKVSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| hIgG2 variant | ERKCCVECPPCPAPPVAGPSVFLFPPKPKDTLMISRTPEVTCVVVDVSHEDP | 150 |
| | EVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPS | |
| | DIAVEWESNGQPENNYKTTPPMLDSDGSFFLYSKLTVDKSRWQQGNVFSCSV | |
| | MHEALHNHYTQKSLSLSPGK | |
| hIgG4 S108P | ESKYGPPCPPCPAPEFLGGPSVFLFPPKPKDTLMISRTPEVTCVVVDVSQED | 151 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCS | |
| | VMHEALHNHYTQKSLSLSLGK | |

6.7.1.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IFN receptor antagonist half antibody) or the Fc region (e.g., one or both Fc domains of an IFN receptor antagonist that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table H below: In some embodiments, the Fc domain includes only the bolded portion of the sequences shown below:

TABLE H

| Fc Domain | Sequence |
|---|---|
| SEQ ID NO: 1 of | Asp Lys Arg Val Glu Ser Lys Tyr Gly Pro Cys Pro Pro Cys |
| WO2014/121087 | Pro Ala Pro Pro Val Ala Gly Pro Ser Val Phe Leu Phe Pro |
| | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly Leu |
| | Pro Ser Ser Ile Glu Lys Thr Ile Ser Lys Ala Lys Gly Gln Pro |
| | Arg Glu Pro Gln Val Tyr Thr Leu Pro Pro Ser Gln Glu Glu |
| | Met Thr Lys Asn Gln Val Ser Leu Thr Cys Leu Val Lys Gly |
| | Phe Tyr Pro Ser Asp Ile Ala Val Glu Trp Glu Ser Asn Gly |
| | Gln Pro Glu Asn Asn Tyr Lys Thr Thr Pro Pro Val Leu Asp |
| | Ser Asp Gly Ser Phe Phe Leu Tyr Ser Arg Leu Thr Val Asp |
| | Lys Ser Arg Trp Gln Glu Gly Asn Val Phe Ser Cys Ser Val |
| | Met His Glu Ala Leu His Asn His Tyr Thr Gln Lys Ser Leu |
| | Ser Leu Ser Leu Gly Lys (SEQ ID NO: 115) |
| SEQ ID NO: 2 of | Asp Lys Lys Val Glu Pro Lys Ser Cys Asp Lys Thr His Thr |
| WO2014/121087 | Cys Pro Pro Cys Pro Ala Pro Pro Val Ala Gly Pro Ser Val |
| | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala |
| | Lys Gly Gln Pro Arg Glu Pro Gln Val Tyr Thr Leu Pro Pro |
| | Ser Arg Asp Glu Leu Thr Lys Asn Gln Val Ser Leu Thr Cys |
| | Leu Val Lys Gly Phe Tyr Pro Ser Asp Ile Ala Val Glu Trp Glu |
| | Ser Asn Gly Gln Pro Glu Asn Asn Tyr Lys Thr Thr Pro Pro |
| | Val Leu Asp Ser Asp Gly Ser Phe Phe Leu Tyr Ser Lys Leu |
| | Thr Val Asp Lys Ser Arg Trp Gln Gln Gly Asn Val Phe Ser |
| | Cys Ser Val Met His Glu Ala Leu His Asn His Tyr Thr Gln |
| | Lys Ser Leu Ser Leu Ser Pro Gly Lys (SEQ ID NO: 116) |
| SEQ ID NO: 30 of | Ala Ser Thr Lys Gly Pro Ser Val Phe Pro Leu Ala Pro Ser |
| WO2014/121087 | Ser Lys Ser Thr Ser Gly Gly Thr Ala Ala Leu Gly Cys Leu |
| | Val Lys Asp Tyr Phe Pro Glu Pro Val Thr Val Ser Trp Asn |
| | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Gln Thr Tyr Ile Cys Asn Val |
| | Asn His Lys Pro Ser Asn Thr Lys Val Asp Lys Lys Val Glu |
| | Pro Lys Ser Cys Asp Lys Thr His Thr Cys Pro Pro Cys |
| | Pro Ala Pro Pro Val Ala Gly Pro Ser Val Phe Leu Phe Pro |
| | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser Ile Glu Lys Thr Ile Ser Lys Ala Lys |
| | Gly Gln Pro Arg Glu Pro Gln Val Tyr Thr Leu Pro Pro Ser |
| | Arg Asp Glu Leu Thr Lys Asn Gln Val Ser Leu Thr Cys |
| | Leu Val Lys Gly Phe Tyr Pro Ser Asp Ile Ala Val Glu Trp |
| | Glu Ser Asn Gly Gln Pro Glu Asn Asn Tyr Lys Thr Thr |
| | Pro Pro Val Leu Asp Ser Asp Gly Ser Phe Phe Leu Tyr |
| | Ser Lys Leu Thr Val Asp Lys Ser Arg Trp Gln Gln Gly |
| | Asn Val Phe Ser Cys Ser Val Met His Glu Ala Leu His |
| | Asn His Tyr Thr Gln Lys Ser Leu Ser Leu Ser Pro Gly |
| | Lys (SEQ ID NO: 117) |
| SEQ ID NO: 31 of | Ala Ser Thr Lys Gly Pro Ser Val Phe Pro Leu Ala Pro Cys |
| WO2014/121087 | Ser Arg Ser Thr Ser Glu Ser Thr Ala Ala Leu Gly Cys Leu |
| | Val Lys Asp Tyr Phe Pro Glu Pro Val Thr Val Ser Trp Asn |
| | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala |
| | Pro Pro Val Ala Gly Pro Ser Val Phe Leu Phe Pro Pro |
| | Lys Pro Lys Asp Thr Leu Met Ile Ser Arg Thr Pro Glu Val |
| | Thr Cys Val Val Val Asp Val Ser Gln Glu Asp Pro Glu Val |
| | Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser Ile Glu Lys Thr Ile Ser Lys Ala Lys |
| | Gly Gln Pro Arg Glu Pro Gln Val Tyr Thr Leu Pro Pro Ser |
| | Gln Glu Glu Met Thr Lys Asn Gln Val Ser Leu Thr Cys |
| | Leu Val Lys Gly Phe Tyr Pro Ser Asp Ile Ala Val Glu Trp |
| | Glu Ser Asn Gly Gln Pro Glu Asn Asn Tyr Lys Thr Thr |
| | Pro Pro Val Leu Asp Ser Asp Gly Ser Phe Phe Leu Tyr |
| | Ser Arg Leu Thr Val Asp Lys Ser Arg Trp Gln Glu Gly |
| | Asn Val Phe Ser Cys Ser Val Met His Glu Ala Leu His |
| | Asn His Tyr Thr Gln Lys Ser Leu Ser Leu Ser Leu Gly |
| | Lys (SEQ ID NO: 118) |
| SEQ ID NO: 37 of | Ala Ser Thr Lys Gly Pro Ser Val Phe Pro Leu Ala Pro Ser |
| WO2014/121087 | Ser Lys Ser Thr Ser Gly Gly Thr Ala Ala Leu Gly Cys Leu |
| | Val Lys Asp Tyr Phe Pro Glu Pro Val Thr Val Ser Trp Asn |
| | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Gln Thr Tyr Ile Cys Asn Val |
| | Asn His Lys Pro Ser Asn Thr Lys Val Asp Lys Lys Val Glu |
| | Pro Lys Ser Cys Asp Lys Thr His Thr Cys Pro Pro Cys |
| | Pro Ala Pro Pro Val Ala Gly Pro Ser Val Phe Leu Phe Pro |
| | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser Ile Glu Lys Thr Ile Ser Lys Ala Lys |
| | Gly Gln Pro Arg Glu Pro Gln Val Tyr Thr Leu Pro Pro Ser |
| | Arg Asp Glu Leu Thr Lys Asn Gln Val Ser Leu Thr Cys |
| | Leu Val Lys Gly Phe Tyr Pro Ser Asp Ile Ala Val Glu Trp |
| | Glu Ser Asn Gly Gln Pro Glu Asn Asn Tyr Lys Thr Thr |
| | Pro Pro Val Leu Asp Ser Asp Gly Ser Phe Phe Leu Tyr |
| | Ser Lys Leu Thr Val Asp Lys Ser Arg Trp Gln Gln Gly |
| | Asn Val Phe Ser Cys Ser Val Met His Glu Ala Leu His |
| | Asn Arg Phe Thr Gln Lys Ser Leu Ser Leu Ser Pro Gly |
| | Lys (SEQ ID NO: 119) |
| SEQ ID NO: 38 of | Ala Ser Thr Lys Gly Pro Ser Val Phe Pro Leu Ala Pro Cys |
| WO2014/121087 | Ser Arg Ser Thr Ser Glu Ser Thr Ala Ala Leu Gly Cys Leu |
| | Val Lys Asp Tyr Phe Pro Glu Pro Val Thr Val Ser Trp Asn |
| | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala |
| | Pro Pro Val Ala Gly Pro Ser Val Phe Leu Phe Pro Pro |
| | Lys Pro Lys Asp Thr Leu Met Ile Ser Arg Thr Pro Glu Val |
| | Thr Cys Val Val Val Asp Val Ser Gln Glu Asp Pro Glu Val |
| | Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser Ile Glu Lys Thr Ile Ser Lys Ala Lys |
| | Gly Gln Pro Arg Glu Pro Gln Val Tyr Thr Leu Pro Pro Ser |
| | Gln Glu Glu Met Thr Lys Asn Gln Val Ser Leu Thr Cys |
| | Leu Val Lys Gly Phe Tyr Pro Ser Asp Ile Ala Val Glu Trp |
| | Glu Ser Asn Gly Gln Pro Glu Asn Asn Tyr Lys Thr Thr |
| | Pro Pro Val Leu Asp Ser Asp Gly Ser Phe Phe Leu Tyr |
| | Ser Arg Leu Thr Val Asp Lys Ser Arg Trp Gln Glu Gly |
| | Asn Val Phe Ser Cys Ser Val Met His Glu Ala Leu His |
| | Asn Arg Phe Thr Gln Lys Ser Leu Ser Leu Ser Leu Gly |
| | Lys (SEQ ID NO: 120) |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.7.1.2. Fc Heterodimerization Variants

Certain IFN receptor antagonists entail dimerization between two Fc domains that, unlike a native immunoglobulin, are operably linked to non-identical N-terminal or C-terminal regions. Inadequate heterodimerization of two Fc domains to form an Fc region has can be an obstacle for increasing the yield of desired heterodimeric molecules and represents challenges for purification. A variety of approaches available in the art can be used in for enhancing dimerization of Fc domains that might be present in the IFN receptor antagonists of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides IFN receptor antagonists comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired IFN receptor antagonist, while homodimerization of identical heavy chains will reduce yield of the desired IFN receptor antagonist. Thus, in a preferred embodiment, the polypeptides that associate to form an IFN receptor antagonist of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IFN receptor antagonists comprise a first CH3 domain and a second Ig CH3 domain, wherein the first and second Ig CH3 domains differ from one another by at least one amino acid, and wherein at least one amino acid difference reduces binding of the IFN receptor antagonist to Protein A as compared to a corresponding IFN receptor antagonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.7.1.3. Hinge Domains

The IFN receptor antagonists of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric IFN receptor antagonist formed by the association of two Fc domains) can comprise two associated hinge sequences on separate polypeptide chains. Sometimes, the two associated hinge sequences are referred to as a “hinge region”. In certain embodiments of IFN receptor antagonists, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an IFN receptor antagonist of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the IFN receptor antagonists of the disclosure comprise a modified hinge region that reduces binding affinity for an Fcγ receptor relative to a wild-type hinge region of the same isotype (e.g., human IgG1 or human IgG4).

In one embodiment, the IFN receptor antagonists of the disclosure comprise an Fc region in which each Fc domain possesses an intact hinge domain at its N-terminus, where each Fc domain and hinge domain is derived from IgG4, and each hinge domain comprises the modified sequence CPPC (SEQ ID NO:121). The core hinge region of human IgG4 contains the sequence CPSC (SEQ ID NO:122) compared to IgG1 that contains the sequence CPPC (SEQ ID NO:121). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.7.1.3.1. Chimeric Hinge Sequences

The hinge domain can be a chimeric hinge domain.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO:123; previously disclosed as SEQ ID NO:8 of WO2014/121087, which is incorporated by reference in its entirety herein) or ESKYGPPCPPCPAPPVA (SEQ ID NO:124; previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.7.1.1).

6.7.1.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO:125; previously disclosed as SEQ ID NO:1 of WO2016161010A2), CPPCPAPGG--GPSVF (SEQ ID NO:126; previously disclosed as SEQ ID NO:2 of WO2016161010A2), CPPCPAPG---GPSVF (SEQ ID NO:127; previously disclosed as SEQ ID NO:3 of WO2016161010A2), or CPPCPAP----GPSVF (SEQ ID NO:128; previously disclosed as SEQ ID NO:4 of WO2016161010A2).

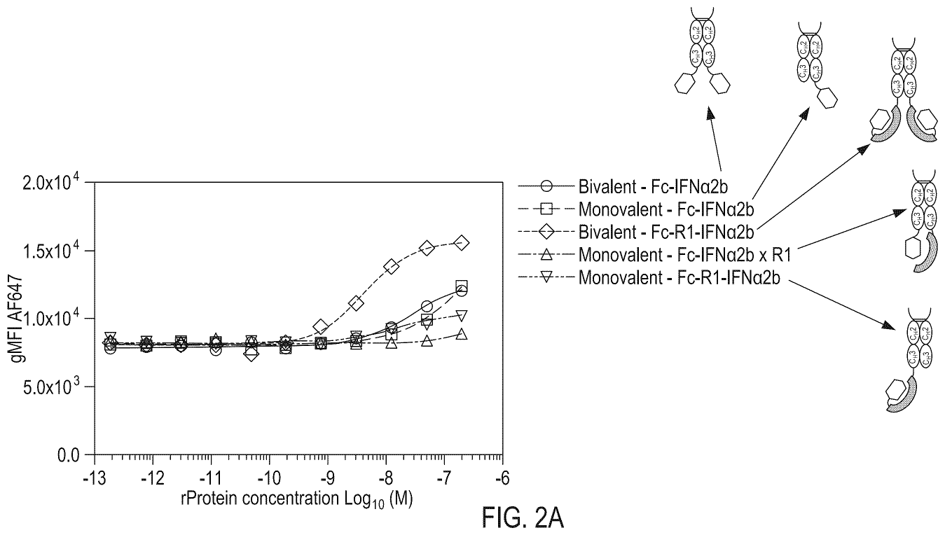

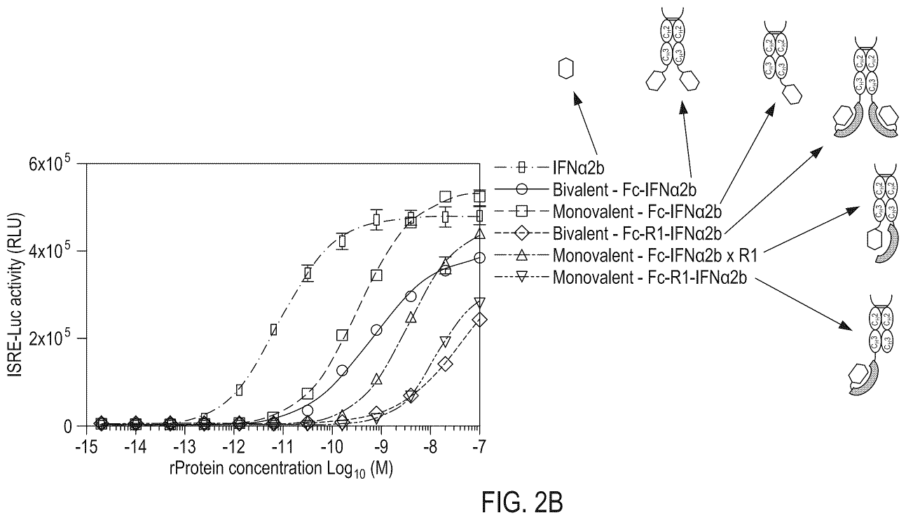

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.7.1.1).

### 6.8. Nucleic Acids and Host Cells

In another aspect, the disclosure provides nucleic acids encoding the IFN receptor antagonists of the disclosure. In some embodiments, the IFN receptor antagonists are encoded by a single nucleic acid. In other embodiments, the IFN receptor antagonists can be encoded by a plurality (e.g., two, three, four or more) nucleic acids.

A single nucleic acid can encode an IFN receptor antagonist that comprises a single polypeptide chain, an IFN receptor antagonist that comprises two or more polypeptide chains, or a portion of an IFN receptor antagonist that comprises more than two polypeptide chains (for example, a single nucleic acid can encode two polypeptide chains of an IFN receptor antagonist comprising three, four or more polypeptide chains, or three polypeptide chains of an IFN receptor antagonist comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, an IFN receptor antagonist comprising two or more polypeptide chains is encoded by two or more nucleic acids. The number of nucleic acids encoding an IFN receptor antagonist can be equal to or less than the number of polypeptide chains in the IFN receptor antagonist (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.8.1. Vectors

The disclosure provides vectors comprising nucleotide sequences encoding an IFN receptor antagonist or a component thereof described herein, for example one or two of the polypeptide chains of a half antibody of an IFN receptor antagonist. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

### 6.8.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.9. Pharmaceutical Compositions

The IFN receptor antagonists of the disclosure may be in the form of compositions comprising the IFN receptor antagonist and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the IFN receptor antagonist and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular IFN receptor antagonist, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an IFN receptor antagonist of the disclosure per dose. The quantity of IFN receptor antagonist included in a unit dose will depend on the disease being treated, as well as other factors as are well known in the art. Such unit dosages may be in the form of a lyophilized dry powder containing an amount of IFN receptor antagonist suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of IFN receptor antagonist suitable for a single administration.

The pharmaceutical compositions may also be supplied in bulk from containing quantities of IFN receptor antagonist suitable for multiple administrations.

Pharmaceutical compositions may be prepared for storage as lyophilized formulations or aqueous solutions by mixing an IFN receptor antagonist having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of IFN receptor antagonist.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The IFN receptor antagonists of the disclosure can be formulated as pharmaceutical compositions comprising the IFN receptor antagonists, for example containing one or more pharmaceutically acceptable excipients or carriers. To prepare pharmaceutical or sterile compositions comprising the IFN receptor antagonists of the present disclosure, a IFN receptor antagonist preparation can be combined with one or more pharmaceutically acceptable excipient or carrier.

For example, formulations of IFN receptor antagonists can be prepared by mixing IFN receptor antagonists with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A composition of the present disclosure may also be administered via one or more routes of administration using one or more of a variety of methods known in the art. As will be appreciated by the skilled artisan, the route and/or mode of administration will vary depending upon the desired results. Selected routes of administration for IFN receptor antagonists include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, the IFN receptor antagonists are administered by infusion. In another embodiment, the IFN receptor antagonist of the disclosure is administered subcutaneously.

### 6.9.1. Pharmaceutical Compositions for Delivery of IFN Receptor Antagonist-Encoding Nucleic Acids

An IFN receptor antagonist of the disclosure can be delivered by means of a nucleic acid encoding the IFN receptor antagonist, for example as a plasmid, DNA, mRNA or through viral vectors encoding the IFN receptor antagonist under the control of a suitable promoter.

In one embodiment, the delivery vector is a virus, including a retrovirus, adenovirus, herpes simplex virus, pox virus, vaccinia virus, lentivirus, or an adeno-associated virus. In one embodiment, the delivery vector is an adeno-associated virus (AAV), including serotypes AAV1, AAV2, AAV3, AAV4, AAV5, AAV6, AAV7, AAV8, AAV9, AAV10, and AAV11, or engineered or naturally selected variants thereof.

Exemplary viral vectors include recombinant adenovirus and adeno-associated virus vectors (rAAV). rAAV vectors are based on the defective and nonpathogenic parvovirus adeno-associated type 2 virus. Most such vectors are derived from a plasmid that retains only the AAV inverted terminal repeats flanking the transgene expression cassette. Efficient gene transfer and stable transgene delivery due to integration into the genomes of the transduced cell are key features for this vector system. AAV serotypes useful for delivering IL27 transgenes AAV1, AAV2, AAV3, AAV4, AAV5, AAV6, AAV8, AAV 8.2, AAV9, and AAV rh10 and pseudotyped AAV such as AAV2/8, AAV2/5 and AAV2/6.

In one embodiment, a nucleic acid encoding an IFN receptor antagonist (or component thereof) also contains adeno-associated virus (AAV) nucleic acid sequence. In one embodiment, the vector is a chimeric adeno-associated virus containing genetic elements from two or more serotypes. For example, an AAV vector with rep genes from AAV1 and cap genes from AAV2 (designated as AAV1/2 or AAV RC1/2) may be used as a delivery vector to deliver an IFN receptor antagonist expressing nucleic acid to a cell or a cell of a patient in need. In one embodiment, the delivery vector is an AAV1/2, AAV1/3, AAV1/4, AAV1/5, AAV1/6, AAV1/7, AAV1/8, AAV1/9, AAV1/10, AAV1/11, AAV2/1, AAV2/3, AAV2/4, AAV2/5, AAV2/6, AAV2/7, AAV2/8, AAV2/9, AAV2/10, AAV2/11, AAV3/1, AAV3/2, AAV3/4, AAV3/5, AAV3/6, AAV3/7, AAV3/8, AAV3/9, AAV3/10, AAV3/10, AAV4/1, AAV4/2, AAV4/3, AAV4/5, AAV4/6, AAV4/7, AAV4/8, AAV4/9, AAV4/10, AAV4/11, AAV5/1, AAV5/2, AAV5/3, AAV5/4, AAV5/6, AAV5/7, AAV5/8, AAV5/9, AAV5/10, AAV5/11, AAV6/1, AAV6/2, AAV6/3, AAV6/4, AAV6/5, AAV6/7, AAV6/8, AAV6/9, AAV6/10, AAV6/10, AAV7/1, AAV7/2, AAV7/3, AAV7/4, AAV7/5, AAV7/6, AAV7/8, AAV7/9, AAV7/10, AAV7/11, AAV8/1, AAV8/2, AAV8/3, AAV8/4, AAV8/5, AAV8/6, AAV8/7, AAV8/9, AAV8/10, AAV8/11, AAV9/1, AAV9/2, AAV9/3, AAV9/4, AAV9/5, AAV9/6, AAV9/7, AAV9/8, AAV9/10, AAV9/11, AAV10/1, AAV10/2, AAV10/3, AAV10/4, AAV10/5, AAV10/6, AAV10/7, AAV10/8, AAV10/9, AAV10/11, AAV11/1, AAV11/2, AAV11/3, AAV11/4, AAV11/5, AAV11/6, AAV11/7, AAV11/8, AAV11/9, AAV11/10, chimeric viral vector, or derivative thereof. Gao et al., “Novel adeno-associated viruses from rhesus monkeys as vectors for human gene therapy,” PNAS 99(18): 11854-11859, Sep. 3, 2002, is incorporated herein by reference for AAV vectors and chimeric viral vectors useful as delivery vectors, and their construction and use.

AAV may be manufactured at a clinical scale by a number of different processes. Examples of systems that can be used include (1) plasmid DNA transfection in mammalian cells, (2) Ad infection of stable mammalian cell lines, (3) infection of mammalian cells with recombinant herpes simplex viruses (rHSVs), and (4) infection of insect cells (Sf9 cells) with recombinant baculoviruses (reviewed by Penaud-Budloo et al., 2018, Mol Ther Methods Clin Dev. 8: 166-180).

Replication-deficient recombinant adenoviral vectors (Ad) can be produced at high titer and readily infect a number of different cell types. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Packaging cells are used to form virus particles that are capable of infecting a host cell. Such cells include 293 cells, which package adenovirus, and w2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

The nucleic acid molecule (e.g., mRNA) or virus can be formulated as the sole pharmaceutically active ingredient in a pharmaceutical composition or can be combined with other active agents for the particular disorder treated. Optionally, other medicinal agents, pharmaceutical agents, carriers, adjuvants, diluents can be included in the compositions provided herein. For example, any one or more of wetting agents, emulsifiers and lubricants, such as sodium lauryl sulfate and magnesium stearate, as well as coloring agents, release agents, coating agents, sweetening, flavoring and perfuming agents, preservatives, antioxidants, chelating agents and inert gases also can be present in the compositions. Exemplary other agents and excipients that can be included in the compositions include, for example, water soluble antioxidants, such as ascorbic acid, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid and phosphoric acid.

### 6.10. Therapeutic Indications and Methods of Use

The present disclosure provides methods for using and applications for the IFN receptor antagonists of the disclosure.

The IFN receptor antagonists of the disclosure can be used to regulate the immune response in a variety of applications.

In certain aspects, the disclosure provides a method of treating cancer or an inflammatory or immune (e.g., autoimmune) disease, comprising administering to a subject in need thereof an IFN receptor antagonist or pharmaceutical composition as described herein, where the IFN receptor antagonist comprises an anchoring moiety (e.g., a targeting moiety) that binds to a target molecule that is present on the surface of a target cell expressing a Type I interferon receptor and associated with a disease.

The present disclosure further provides method of locally regulating an immune response in a target tissue, comprising administering to a subject IFN receptor antagonist or pharmaceutical composition as described herein which has one or more anchoring moieties (e.g., targeting moieties) capable of binding a target molecule expressed in the target tissue.

In some embodiments, the administration is not local to the tissue. For example, when the target tissue is cancer tissue, the administration can be systemic or subcutaneous.

The IFN receptor antagonists of the disclosure can be used to treat autoimmune inflammatory diseases, such as systemic lupus erythematosus (SLE), rheumatoid arthritis (RA), e.g., by reducing a local or tissue-specific autoimmune response.

The IFN receptor antagonists of the disclosure can be used in the treatment of any proliferative disorder (e.g., cancer) that expresses a target molecule (either on the tumor cells or in the tumor microenvironment, e.g., the extracellular matrix or the tumor lymphocytes). In particular embodiments, the IFN antagonists of the disclosure are administered to subjects undergoing oncolytic virotherapy, who previously underwent oncolytic virotherapy, or who will be undergoing oncolytic virotherapy.

In some embodiments, the cancer is acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor.

Table I below shows exemplary indications for which IFN receptor antagonists targeting particular target molecules can be used.

TABLE I
Examples of Target Molecule Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, Kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Additional target molecules and corresponding indications are disclosed in, e.g., Hafeez et al., 2020, Molecules 25:4764, doi: 10.3390/molecules25204764, particularly in Table 1. Table 1 is incorporated by reference in its entirety here.

In further embodiments, the IFN receptor antagonists can be used to regulate an immune response elicited by another agent. Thus, in some embodiments an IFN receptor antagonist of the disclosure is administered as an adjunct therapy with an immunogenic agent. In some embodiments, the immunogenic agent is an adjuvanted or unadjuvanted vaccine. The IFN receptor antagonists can thus enhance an antigen-specific immune response elicited by the vaccine. In various embodiments, the vaccine is a prophylactic or therapeutic cancer vaccine or a prophylactic or therapeutic vaccine against an infectious agent, e.g., a virus, bacteria, or parasite.

### 6.10.1. Combination Therapy

The IFN receptor antagonists according to the disclosure may be administered in combination with one or more other agents in therapy. For instance, a IFN receptor antagonist of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other.

Without being bound by theory, it is believed that administration of an IFN receptor antagonist of the disclosure in combination with oncolytic virus therapy allows for enhanced efficacy and reduced side effects of the oncolytic virus treatment by virtue of inhibition of IFN signaling caused by upregulation of type I IFNs in response to oncolytic virus administration. Accordingly, in some embodiments, the additional therapeutic agent is an oncolytic virus.

In certain embodiments, an additional therapeutic agent is an immunosuppressive agent, including but not limited to mycophenolate mofetil (MMF), mycophenolic acid (MPA), cyclosporin A, FK506-like compounds (e.g. FK506, FK506 derivatives, and FK506 analogs), rapamycin compounds (including rapamycin, rapamycin derivatives, and rapamycin analogs), corticosteroids (e.g., hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof), non-steroidal anti-inflammatory agents (e.g., oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone), and anti-inflammatory cytokines or chemokines (e.g., IL-4, IL-6, IL-10, IL-11, and IL-13).

Such other agents are suitably present in combination in amounts that are effective for the purpose intended. The effective amount of such other agents depends on the amount of IFN receptor antagonist used, the type of disorder or treatment, and other factors discussed above. The IFN receptor antagonists are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the IFN receptor antagonist of the disclosure can occur prior to, simultaneously, and/or following, administration of the additional therapeutic agent and/or adjuvant.

### 7. SEQUENCES

Certain sequences of the disclosure are provided in Table S below.

TABLE S
Sequences

| | | SEQ ID |
|---|---|---|
| Name | Sequence | NO: |
| human | MASPFALLMVLVVLSCKSSCSLGCDLPETHSLDNRRTLMLLAQMSRISPS | 1 |
| IFNα1a | SCLMDRHDFGFPQEEFDGNQFQKAPAISVLHELIQQIFNLFTTKDSSAAW | |
| precursor | DEDLLDKFCTELYQQLNDLEACVMQEERVGETPLMNADSILAVKKYFRRI | |
| | TLYLTEKKYSPCAWEVVRAEIMRSLSLSTNLQERLRRKE | |
| human | MALTFALLVALLVLSCKSSCSVGCDLPQTHSLGSRRTLMLLAQMRRISLE | 2 |
| IFNα2 | SCLKDRHDFGFPQEEFGNQFQKAETIPVLHEMIQQIFNLFSTKDSSAAWD | |
| precursor | ETLLDKFYTELYQQLNDLEACVIQGVGVTETPLMKEDSILAVRKYFQRIT | |
| | LYLKEKKYSPCAWEVVRAEIMRSFSLSTNLQESLRSKE | |
| IFNα1a | CDLPETHSLDNRRTLMLLAQMSRISPSSCLMDRHDFGFPQEEFDGNQFQK | 3 |
| | APAISVLHELIQQIFNLFTTKDSSAAWDEDLLDKFCTELYQQLNDLEACV | |
| | MQEERVGETPLMNADSILAVKKYFRRITLYLTEKKYSPCAWEVVRAEIMR | |
| | SLSLSTNLQERLRRKE | |
| IFNα1b | CDLPETHSLDNRRTLMLLAQMSRISPSSCLMDRHDFGFPQEEFDGNQFQK | 4 |
| | APAISVLHELIQQIFNLFTTKDSSAAWDEDLLDKFCTELYQQLNDLEACV | |
| | MQEERVGETPLMNVDSILAVKKYFRRITLYLTEKKYSPCAWEVVRAEIMR | |
| | SLSLSTNLQERLRRKE | |
| IFNα2a | CDLPQTHSLGSRRTLMLLAQMRKISLESCLKDRHDFGFPQEEFGNQFQKA | 5 |
| | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMRS | |
| | FSLSTNLQESLRSKE | |
| IFNα2b | CDLPQTHSLGSRRTLMLLAQMRRISLESCLKDRHDFGFPQEEFGNQFQKA | 6 |
| | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMRS | |
| | FSLSTNLQESLRSKE | |
| IFNα2b | CDLPQTHSLGSRRTLMLLAQMRRISLESCLKDAHDFGFPQEEFGNQFQKA | 7 |
| (R33A) | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMRS | |
| | FSLSTNLQESLRSKE | |
| IFNα2b | CDLPQTHSLGSRRTLMLLAQMRRISLESCLKDKHDFGFPQEEFGNQFQKA | 8 |
| (R33K) | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMRS | |
| | FSLSTNLQESLRSKE | |
| IFNα2b | CDLPQTHSLGSRRTLMLLAQMRRISLFSCLKDRHDFGFPQEEFGNQFQKA | 9 |
| (Q90A) | ETIPVLHEMIQQIFNLFSTKDSSAAWDETLLDKFYTELYAQLNDLEACVI | |
| | QGVGVTETPLMKEDSILAVRKYFQRITLYLKEKKYSPCAWEVVRAEIMRS | |
| | FSLSTNLQESLRSKE | |
| IFNα2b | CDLPQTHSLGSRRTLMLLAQMRRISLFSCLKDRHDFGFPQEEFGNQFQKA | 10 |
| (E96A) | ETIPVLHEMIQQIFNLFSTKDSSAAWDETLLDKFYTELYQQLNDLAACVI | |
| | QGVGVTETPLMKEDSILAVRKYFQRITLYLKEKKYSPCAWEVVRAEIMRS | |
| | FSLSTNLQESLRSKE | |
| IFNα2b | CDLPQTHSLGSRRTLMLLAQMRRISLFSCLKDRHDFGFPQEEFGNQFQKA | 11 |
| (R120A) | ETIPVLHEMIQQIFNLFSTKDSSAAWDETLLDKFYTELYQQLNDLEACVI | |
| | QGVGVTETPLMKEDSILAVAKYFQRITLYLKEKKYSPCAWEVVRAEIMRS | |
| | FSLSTNLQESLRSKE | |
| IFNα2b | CDLPQTHSLGSRRTLMLLAQMRRISLFSCLKDRHDFGFPQEEFGNQFQKA | 12 |
| (A145M) | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRMEIMRS | |
| | FSLSTNLQESLRSKE | |
| IFNα2b | CDLPQTHSLGSRRTLMLLAQMRRISLFSCLKDRHDFGFPQEEFGNQFQKA | 13 |
| (R149A) | ETIPVLHEMIQQIENLESTKDSSAAWDETLLDKFYTELYQQLNDLEACVI | |
| | QGVGVTETPLMKEDSILAVRKYFQRITLYLKEKKYSPCAWEVVRAEIMAS | |
| | FSLSTNLQESLRSKE | |
| IFNα2b | CDLPQTHSLGSRRTLMLLAQMRRISLFSCLKDRHDFGFPQEEFGNQFQKA | 14 |
| (R149K) | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMKS | |
| | FSLSTNLQESLRSKE | |
| IFNα2b | CDLPQTHSLGSRRTLMLLAQMRRISLFSCLKDRHDFGFPQEEFGNQFQKA | 15 |
| (S152A) | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMRS | |
| | FALSTNLQESLRSKE | |
| ΔN-IFN- | THSLGSRRTLMLLAQMRRISLFSCLKDRHDFGFPQEEFGNQFQKAETIPV | 16 |
| ΔC | LHEMIQQIFNLFSTKDSSAAWDETLLDKFYTELYQQLNDLEACVIQGVGV | |
| | TETPLMKEDSILAVRKYFQRITLYLKEKKYSPCAWEVVRAEIMRSESLST | |
| | NLQ | |
| IFN-ΔC | CDLPQTHSLGSRRTLMLLAQMRRISLESCLKDRHDFGFPQEEFGNQFQKA | 17 |
| | ETIPVLHEMIQQIFNLFSTKDSSAAWDETLLDKFYTELYQQLNDLEACVI | |
| | QGVGVTETPLMKEDSILAVRKYFQRITLYLKEKKYSPCAWEVVRAEIMRS | |
| | FSLSTNLQ | |
| human | MTNKCLLQIA LLLCESTTAL SMSYNLLGfL QRSSNFQCQK | 18 |
| IFNß | LLWQLNGRLE YCLKDRMNfD IPEEIKQLQQ FQKEDAALTI | |
| precursor | YEMLQNIFAI FRQDSSSTGW NETIVENLLA NVYHQINHLK | |
| | TVLEEKLEKE DfTRGKLMSS LHLKRYYGRI LHYLKAKEYS | |
| | HCAWTIVRVE ILRNFYFINR LTGYLRN | |
| Human | MALLFPLLAALVMTSYSPVGSLGCDLPQNHGLLSRNTLVLLHQMRRISPE | 19 |
| IFNω | LCLKDRRDFRFPQEMVKGSQLQKAHVMSVLHEMLQQIFSLFHTERSSAAW | |
| precursor | NMTLLDQLHTGLHQQLQHLETCLLQVVGEGESAGAISSPALTLRRYFQGI | |
| | RVYLKEKKYSDCAWEVVRMEIMKSLFLSTNMQERLRSKDRDLGSS | |
| Human | MIIKHFFGTVLVLLASTTIFSLDLKLIIFQQRQVNQESLKLLNKLQTLSI | 20 |
| IFNϵ | QQCLPHRKNFLLPQKSLSPQQYQKGHTLAILHEMLQQIFSLFRANISLDG | |
| precursor | WEENHTEKFLIQLHQQLEYLEALMGLEAEKLSGTLGSDNLRLQVKMYERR | |
| | IHDYLENQDYSTCAWAIVQVEISRCLFFVESLTEKLSKQGRPLNDMKQEL | |
| | TTEFRSPR | |
| Human | MSTKPDMIQKCLWLEILMGIFIAGTLSLDCNLLNVHLRRVTWQNLRHLSS | 21 |
| IFNκ | MSNSFPVECLRENIAFELPQEFLQYTQPMKRDIKKAFYEMSLQAFNIFSQ | |
| precursor | HTFKYWKERHLKQIQIGLDQQAEYLNQCLEEDKNENEDMKEMKENEMKPS | |
| | EARVPQLSSLELRRYFHRIDNFLKEKKYSDCAWEIVRVEIRRCLYYFYKE | |
| | TALERRK | |
| human | MMVVLLGATTLVLVAVAPWVLSAAAGGKNLKSPQKVEVDIIDDNFILRWN | 22 |
| IFNAR1 | RSDESVGNVTFSEDYQKTGMDNWIKLSGCQNITSTKCNFSSLKLNVYEEI | |
| precursor | KLRIRAEKENTSSWYEVDSFTPFRKAQIGPPEVHLEAEDKAIVIHISPGT | |
| | KDSVMWALDGLSFTYSLVIWKNSSGVEERIENIYSRHKIYKLSPETTYCL | |
| | KVKAALLTSWKIGVYSPVHCIKTTVENELPPPENIEVSVQNQNYVLKWDY | |
| | TYANMTFQVQWLHAFLKRNPGNHLYKWKQIPDCENVKTTQCVFPQNVFQK | |
| | GIYLLRVQASDGNNTSFWSEEIKEDTEIQAFLLPPVENIRSLSDSFHIYI | |
| | GAPKQSGNTPVIQDYPLIYEIIFWENTSNAERKIIEKKTDVTVPNLKPLT | |
| | VYCVKARAHTMDEKLNKSSVESDAVCEKTKPGNTSKIWLIVGICIALFAL | |
| | PFVIYAAKVFLRCINYVFFPSLKPSSSIDEYFSEQPLKNLLLSTSEEQIE | |
| | KCFIIENISTIATVEETNQTDEDHKKYSSQTSQDSGNYSNEDESESKTSE | |
| | ELQQDFV | |
| uIFN | CDLPQTHSLGSRRTLMLLAQMRKISLESCLKDRHDFGFPQEEFGNQFQKA | 137 |
| | ETIPVLHEMIQQIFNLFTTKDSSAAWDEDLLDKFCTELYQQLNDLEACVM | |
| | QEERVGETPLMNVDSILAVKKYFRRITLYLTEKKYSPCAWEVVRAEIMRS | |
| | LSLSTNLQERLRRKE | |

### 8. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below.
1. A Type I interferon (IFN) receptor antagonist, comprising:
   (a) an anchoring moiety for a cell expressing a Type I interferon receptor;
   (b) a Type I interferon alpha receptor 1 (IFNAR1) moiety;
   (c) a Type I interferon (IFN) moiety;
   (d) a separator moiety connecting the anchoring moiety to the IFNAR1 moiety or the IFN moiety; and
   (e) optionally, a linker connecting the IFNAR1 moiety and the IFN moiety.
2. The IFN receptor antagonist of embodiment 1, wherein the anchoring moiety is capable of binding to an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.
3. The IFN receptor antagonist of embodiment 1 or 2, wherein the anchoring moiety is an anchoring moiety for an immune cell.
4. The IFN receptor antagonist of embodiment 2, wherein the anchoring moiety is an anchoring moiety for a B cell.
5. The IFN receptor antagonist of embodiment 1 or 2, wherein the anchoring moiety is an anchoring moiety for a cancer cell.
6. The IFN receptor antagonist of any one of embodiments 1 to 5, wherein the anchoring moiety is a targeting moiety.
7. The IFN receptor antagonist of embodiment 6, wherein the targeting moiety is an scFv or a Fab.
8. The IFN receptor antagonist of embodiment 6 or 7, wherein the targeting moiety is capable of binding to a target molecule identified in Section 6.6.1.
9. The IFN receptor antagonist of any one of embodiments 6 to 8, wherein the targeting moiety (a) comprises the (i) CDR or (ii) VH and VL sequences of antibody set forth in Table F or (b) competes with the antibody set forth in Table F for binding to the target molecule.
10. The IFN receptor antagonist of any one of embodiments 6 to 9, wherein the targeting moiety is capable of binding to a checkpoint inhibitor.
11. The IFN receptor antagonist of embodiment 10, wherein the checkpoint inhibitor is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.
12. The IFN receptor antagonist of embodiment 11, wherein the checkpoint inhibitor is PDL1.
13. The IFN receptor antagonist of embodiment 12, wherein the targeting moiety is an antigen binding fragment of an anti-PDL1 antibody.
14. The IFN receptor antagonist of any one of embodiments 1 to 5, wherein the anchoring moiety is a cell surface protein binding molecule.
15. The IFN receptor antagonist of embodiment 14, wherein the anchoring moiety is PD1 or a PDL1-binding portion thereof.
16. The IFN receptor antagonist of any one of embodiments 1 to 15, wherein the separator moiety is an Fc domain.
17. The IFN receptor antagonist of embodiment 16, wherein the Fc domain comprises a hinge domain.
18. The IFN receptor antagonist of any one of embodiments 1 to 17, wherein the linker is a non-cleavable linker (NCL).
19. The IFN receptor antagonist of embodiment 18, wherein the NCL is a NCL described in Table E.
20. The IFN receptor antagonist of any one of embodiments 1 to 19, further comprising an additional linker connecting the separator moiety to the IFNAR1 moiety.
21. The IFN receptor antagonist of any one of embodiments 1 to 19, further comprising an additional linker connecting the separator moiety to the IFN moiety.
22. The IFN receptor antagonist of embodiment 20 or 21, wherein the additional linker is a NCL.
23. The IFN receptor antagonist of embodiment 22, wherein the additional linker is a NCL described in Table E.
24. The IFN receptor antagonist of any one of embodiments 1 to 23, further comprising an additional anchoring moiety.
25. The IFN receptor antagonist of embodiment 24, wherein the additional anchoring moiety is an additional targeting moiety.
26. The IFN receptor antagonist of embodiment 25, wherein the additional targeting moiety is an scFv or a Fab.
27. The IFN receptor antagonist of embodiment 26, wherein the additional targeting moiety specifically binds to the same target as the anchoring moiety.
28. The IFN receptor antagonist of any one of embodiments 25 to 27, wherein the additional targeting moiety is capable of binding to a target molecule identified in Section 6.6.1.
29. The IFN receptor antagonist of any one of embodiments 25 to 28, wherein the additional targeting moiety (a) comprises the (i) CDR or (ii) VH and VL sequences of antibody set forth in Table F or (b) competes with the antibody set forth in Table F for binding to the target molecule.
30. The IFN receptor antagonist of any one of embodiments 25 to 29, wherein the additional targeting moiety is capable of binding to a checkpoint inhibitor.
31. The IFN receptor antagonist of embodiment 30, wherein the checkpoint inhibitor is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.
32. The IFN receptor antagonist of embodiment 31, wherein the checkpoint inhibitor is PDL1.
33. The IFN receptor antagonist of embodiment 32, wherein the additional targeting moiety is an antigen binding fragment of an anti-PD1 antibody.
34. The IFN receptor antagonist of embodiment 24, wherein the anchoring moiety is a cell surface protein binding molecule.
35. The IFN receptor antagonist of embodiment 34, wherein the anchoring moiety is PD1 or a PDL1-binding portion thereof.
36. The IFN receptor antagonist of any one of embodiments 24 to 35, further comprising an additional separator moiety operably linked to the additional anchoring moiety.
37. The IFN receptor antagonist of embodiment 36, wherein the additional separator moiety is an additional Fc domain.
38. The IFN receptor antagonist of embodiment 37, wherein the additional Fc domain comprises a hinge domain.
39. The IFN receptor antagonist of any one of embodiments 1 to 38, wherein the IFN moiety is masked by the IFNAR1 moiety.
40. The IFN receptor antagonist of any one of embodiments 1 to 39, wherein the IFN receptor antagonist comprises a first polypeptide chain and a second polypeptide chain.
41. The IFN receptor antagonist of embodiment 40, wherein the first polypeptide chain comprises:
   (a) the anchoring moiety or a component thereof;
   (b) the separator moiety; and
   (c) the IFNAR1 moiety.
42. The IFN receptor antagonist of embodiment 41, wherein the second polypeptide chain comprises:
   (a) an additional anchoring moiety or a component thereof; and
   (b) an additional separator moiety.
43. The IFN receptor antagonist of embodiment 41 or 42, wherein the first polypeptide chain further comprises the IFN moiety.
44. The IFN receptor antagonist of embodiment 43, wherein the IFN moiety is C-terminal to the IFNAR1 moiety.
45. The IFN receptor antagonist of embodiment 43, wherein the IFN moiety is N-terminal to the IFNAR1 moiety.
46. The IFN receptor antagonist of any one of embodiments 41 to 45, wherein the IFNAR1 moiety is C-terminal to the separator moiety.
47. The IFN receptor antagonist of any one of embodiments 40 to 43, wherein the first polypeptide chain comprises, in an N- to C-terminal orientation,
   (a) the anchoring moiety or component thereof;
   (b) the separator moiety;
   (c) the IFNAR1 moiety; and
   (d) the IFN moiety.
48. The IFN receptor antagonist of any one of embodiments 40 to 43, wherein the first polypeptide chain comprises, in an N- to C-terminal orientation,
   (a) the anchoring moiety or component thereof;
   (b) the separator moiety;
   (c) the IFN moiety; and
   (d) the IFNAR1 moiety.
49. The IFN receptor antagonist of embodiment 42, wherein the second polypeptide chain further comprises the IFN moiety.
50. The IFN receptor antagonist of embodiment 49, wherein the second polypeptide chain comprises, in an N- to C-terminal orientation:
   (a) an additional anchoring moiety or a component thereof;
   (b) an additional separator moiety; and
   (c) the IFN moiety.
51. The IFN receptor antagonist of any one of embodiments 40 to 48, wherein the second polypeptide chain further comprises an additional IFNAR1 moiety and an additional IFN moiety.
52. The IFN receptor antagonist of any one of embodiments 1 to 51, wherein the IFN moiety comprises an amino acid sequence having at least about 90% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
53. The IFN receptor antagonist of embodiment 52, wherein the IFN moiety comprises an amino acid sequence having at least about 90% sequence identity to (a) full length mature human IFNα2b or (b) a mature human IFNα2b, having up to a 15-amino acid truncation at its N-terminus and/or its C-terminus.
54. The IFN receptor antagonist of any one of embodiments 1 to 51, wherein the IFN moiety comprises an amino acid sequence having at least about 95% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
55. The IFN receptor antagonist of embodiment 54, wherein the IFN moiety comprises an amino acid sequence having at least about 95% sequence identity to (a) full length mature human IFNα2b or (b) a mature human IFNα2b, having up to a 15-amino acid truncation at its N-terminus and/or its C-terminus.
56. The IFN receptor antagonist of any one of embodiments 1 to 51, wherein the IFN moiety comprises an amino acid sequence having at least about 98% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
57. The IFN receptor antagonist of embodiment 56, wherein the IFN moiety comprises an amino acid sequence having at least about 98% sequence identity to (a) full length mature human IFNα2b or (b) a mature human IFNα2b, having up to a 15-amino acid truncation at its N-terminus and/or its C-terminus.
58. The IFN receptor antagonist of any one of embodiments 1 to 51, wherein the IFN moiety is (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
59. The IFN receptor antagonist of embodiment 58, wherein the IFN moiety is (a) full length mature human IFNα2b or (b) a mature human IFNα2b, having up to a 15-amino acid truncation at its N-terminus and/or its C-terminus.
60. The IFN receptor antagonist of any one of embodiments 1 to 59, wherein the IFNAR1 moiety comprises or consists of an amino acid sequence having at least 90% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.
61. The IFN receptor antagonist of any one of embodiments 1 to 59, wherein the IFNAR1 moiety comprises or consists of an amino acid sequence having at least 95% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.
62. The IFN receptor antagonist of any one of embodiments 1 to 59, wherein the IFNAR1 moiety comprises or consists of an amino acid sequence having at least 98% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.
63. The IFN receptor antagonist of any one of embodiments 1 to 59, wherein the IFNAR1 moiety is (i) the SD2 and SD3 domains of human IFNAR1, (ii) the SD1, SD2 and SD3 domains of human IFNAR1, or (iii) the SD1, SD2, SD3 and SD4 domains of human IFNAR1.
64. The IFN receptor antagonist of embodiment 51, wherein the additional IFN moiety comprises an amino acid sequence having at least about 90% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
65. The IFN receptor antagonist of embodiment 64, wherein the additional IFN moiety comprises an amino acid sequence having at least about 90% sequence identity to (a) full length mature human IFNα2b or (b) a mature human IFNα2b, having up to a 15-amino acid truncation at its N-terminus and/or its C-terminus.
66. The IFN receptor antagonist of embodiment 51, wherein the additional IFN moiety comprises an amino acid sequence having at least about 95% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
67. The IFN receptor antagonist of embodiment 66, wherein the additional IFN moiety comprises an amino acid sequence having at least about 95% sequence identity to (a) full length mature human IFNα2b or (b) a mature human IFNα2b, having up to a 15-amino acid truncation at its N-terminus and/or its C-terminus.
68. The IFN receptor antagonist of embodiment 51, wherein the additional IFN moiety comprises an amino acid sequence having at least about 98% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
69. The IFN receptor antagonist of embodiment 68, wherein the additional IFN moiety comprises an amino acid sequence having at least about 98% sequence identity to (a) full length mature human IFNα2b or (b) a mature human IFNα2b, having up to a 15-amino acid truncation at its N-terminus and/or its C-terminus.
70. The IFN receptor antagonist of embodiment 51, wherein the additional IFN moiety is (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.
71. The IFN receptor antagonist of embodiment 70, wherein the additional IFN moiety is (a) full length mature human IFNα2b or (b) a mature human IFNα2b, having up to a 15-amino acid truncation at its N-terminus and/or its C-terminus.
72. The IFN receptor antagonist of embodiment 51, wherein the additional IFNAR1 moiety comprises or consists of an amino acid sequence having at least 90% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.
73. The IFN receptor antagonist of embodiment 51, wherein the additional IFNAR1 moiety comprises or consists of an amino acid sequence having at least 95% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.
74. The IFN receptor antagonist of embodiment 51, wherein the additional IFNAR1 moiety comprises or consists of an amino acid sequence having at least 98% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.
75. The IFN receptor antagonist of embodiment 51, wherein the additional IFNAR1 moiety is (i) the SD2 and SD3 domains of human IFNAR1, (ii) the SD1, SD2 and SD3 domains of human IFNAR1, or (iii) the SD1, SD2, SD3 and SD4 domains of human IFNAR1.
76. The IFN receptor antagonist of any one of embodiments 1 to 75, wherein the IFN receptor antagonist does not comprise an IFNAR2 moiety.
77. The IFN receptor antagonist of embodiment 76, wherein the IFNAR2 moiety comprises or consists of an amino acid sequence having at least 90% sequence identity to (i) the D1 domain of human IFNAR2 or (ii) the D1 and D2 domains of human IFNAR2.
78. The IFN receptor antagonist of embodiment 76, wherein the IFNAR2 moiety comprises or consists of an amino acid sequence having at least 95% sequence identity to (i) the D1 domain of human IFNAR2 or (ii) the D1 and D2 domains of human IFNAR2.
79. The IFN receptor antagonist of embodiment 76, wherein the IFNAR2 moiety comprises or consists of an amino acid sequence having at least 98% sequence identity to (i) the D1 domain of human IFNAR2 or (ii) the D1 and D2 domains of human IFNAR2.
80. The IFN receptor antagonist of embodiment 76, wherein the IFNAR2 moiety is (i) the D1 domain of human IFNAR2 or (ii) the D1 and D2 domains of human IFNAR2.
81. The IFN receptor antagonist of any one of embodiments 1 to 80, which comprises any pair of half-antibodies delineated in Table 2.
82. An IFN receptor antagonist, which is optionally an IFN receptor antagonist according to any one of embodiments 1 to 80, which comprises polypeptide chains having the configuration of the two half-antibodies illustrated in FIG. 1B.
83. An IFN receptor antagonist, which is optionally an IFN receptor antagonist according to any one of embodiments 1 to 80, which comprises polypeptide chains having the configuration of the two half-antibodies illustrated in FIG. 1C.
84. An IFN receptor antagonist, which is optionally an IFN receptor antagonist according to any one of embodiments 1 to 80, which comprises polypeptide chains having the configuration of the two half-antibodies illustrated in FIG. 1D.
85. An IFN receptor antagonist, which is optionally an IFN receptor antagonist according to any one of embodiments 1 to 80, which comprises polypeptide chains having the configuration of the two half-antibodies illustrated in FIG. 1E.
86. An IFN receptor antagonist, which is optionally an IFN receptor antagonist according to any one of embodiments 1 to 80, which comprises polypeptide chains having the configuration of the two half-antibodies illustrated in FIG. 1F.
87. A Type I interferon (IFN) receptor antagonist, which is optionally an IFN receptor antagonist according to any one of embodiments 1 to 86, comprising:
   (a) a first polypeptide comprising, in an N- to C-terminal orientation:
      (i) a first targeting moiety;
      (ii) a first Fc domain;
      (iii) a first linker;
      (iv) an IFNAR1 moiety;
      (v) a second linker; and
      (vi) an IFN moiety; and
   (b) a second polypeptide comprising, in an N- to C-terminal orientation:
      (i) a second targeting moiety; and
      (ii) a second Fc domain associated with the first Fc domain.
88. A Type I interferon (IFN) receptor antagonist, which is optionally an IFN receptor antagonist according to any one of embodiments 1 to 86, comprising:
(a) a first polypeptide comprising, in an N- to C-terminal orientation:
   (i) a first targeting moiety;
   (ii) a first Fc domain;
   (iii) a first linker;
   (iv) an IFN moiety;
   (v) a second linker; and
   (vi) an IFNAR1 moiety; and
(b) a second polypeptide comprising, in an N- to C-terminal orientation:
   (i) a second targeting moiety; and
   (ii) a second Fc domain associated with the first Fc domain.
89. A Type I interferon (IFN) receptor antagonist, which is optionally an IFN receptor antagonist according to any one of embodiments 1 to 86, comprising:
   (a) a first polypeptide comprising, in an N- to C-terminal orientation:
      (i) a first targeting moiety;
      (ii) a first Fc domain;
      (iii) a first linker;
      (iv) a first IFNAR1 moiety;
      (v) a second linker; and
      (vi) a first IFN moiety; and
   (b) a second polypeptide comprising, in an N- to C-terminal orientation:
      (i) a second targeting moiety;
      (ii) a second Fc domain associated with the first Fc domain;
      (iii) a third linker;
      (iv) a second IFNAR1 moiety;
      (v) a fourth linker; and
      (vi) a second IFN moiety.
90. A Type I interferon (IFN) receptor antagonist, which is optionally an IFN receptor antagonist according to any one of embodiments 1 to 86, comprising:
   (a) a first polypeptide comprising, in an N- to C-terminal orientation:
      (i) a first targeting moiety;
      (ii) a first Fc domain;
      (iii) a first linker;
      (iv) a first IFN moiety;
      (v) a second linker; and
      (vi) a first IFNAR1 moiety; and
   (b) a second polypeptide comprising, in an N- to C-terminal orientation:
      (i) a second targeting moiety;
      (ii) a second Fc domain associated with the first Fc domain;
      (iii) a third linker;
      (iv) a second IFN moiety;
      (v) a fourth linker; and
      (vi) a second IFNAR1 moiety.
91. A nucleic acid or plurality of nucleic acids encoding the IFN receptor antagonist of any one of embodiments 1 to 90.
92. A host cell engineered to express the IFN receptor antagonist of any one of embodiments 1 to 90 or the nucleic acid(s) of embodiment 70.
93. A method of producing the IFN receptor antagonist of any one of embodiments 1 to 90, comprising culturing the host cell of embodiment 92, and recovering the IFN receptor antagonist expressed thereby.
94. A pharmaceutical composition comprising the IFN receptor antagonist of any one of embodiments 1 to 90 and an excipient.
95. The pharmaceutical composition of embodiment 94, further comprising an oncolytic virus.
96. A method of inhibiting IFN signaling in a cell expressing (a) an IFN receptor and (b) a molecule to which the anchoring moiety specifically binds, the method comprising contacting the cell with the IFN receptor antagonist of any one of embodiments 1 to 90 or the pharmaceutical composition of embodiment 94.
97. The method of embodiment 96, wherein the cell is an immune cell.
98. The method of embodiment 96 or 97, wherein the cell is a B cell.
99. The method of embodiment 96, wherein the cell is a cancer cell.
100. The method of any one of embodiments 96 to 99, wherein the method is an in vitro method.
101. The method of any one of embodiments 96 to 99, wherein the method comprises administering the IFN receptor antagonist to a subject in need thereof.
102. The method of embodiment 101, wherein the subject is a patient having cancer.
103. The method of embodiment 102, further comprising administering to the subject an oncolytic virus.
104. The method of embodiment 103, wherein the oncolytic virus is an oncolytic vesicular stomatitis virus (VSV).
105. The method of embodiment 101, wherein the subject is a patient having an immune disorder or condition.
106. The method of embodiment 105, wherein the immune disorder or condition is systemic lupus erythematosus (SLE), rheumatoid arthritis (RA), systemic sclerosis (SSc), or Sjogren's syndrome (SS).
107. A method of reducing inflammation, the method comprising administering to a subject in need thereof the IFN receptor antagonist of any one of embodiments 1 to 90 or the pharmaceutical composition of embodiment 94.
108. A method of treating a subject suffering from cancer, the method comprising administering to a subject in need thereof the IFN receptor antagonist of any one of embodiments 1 to 90 or the pharmaceutical composition of embodiment 94.
109. The method of embodiment 108, further comprising administering to the subject an oncolytic virus.
110. The method of embodiment 109, wherein the oncolytic virus is an oncolytic vesicular stomatitis virus (VSV).
111. A method of treating a subject suffering from an immune disorder or condition comprising administering to the subject the IFN receptor antagonist of any one of embodiments 1 to 90 or the pharmaceutical composition of embodiment 94.
112. The method of embodiment 111, wherein the immune disorder or condition is systemic lupus erythematosus (SLE), rheumatoid arthritis (RA), systemic sclerosis (SSc), or Sjogren's syndrome (SS).

### 9. EXAMPLES

### 9.1. Materials and Methods

### 9.1.1. IFN Receptor Antagonist Construct Design

IFN receptor antagonist constructs depicted in FIG. 1 were designed as dimers (e.g., homodimers or heterodimers) comprising two polypeptide chains. Table E-1 below provides core polypeptide sequences utilized in the studies described herein. The core polypeptide sequences in Table E-1 can be utilized to generate IFN receptor antagonist and control constructs whereby a targeting moiety, e.g., a Fab or an scFv, can be added to the N-terminal of each polypeptide chain.

TABLE E-1

| Construct | Sequence |
|---|---|
| Fc-IFN (homodimeric, | Chain 1: |
| both chains identical) | ESKYGPPCPPCPAPPVAGPSVFLFPPKPKDTLMISRTPEVTCVVVDVSQEDP |
| Chain 1: Fc-linker-IFN | EVQFNWYVDGVEVHNAKTKPREEQFNSTYRVVSVLTVLHQDWLNGKEYKCKV |
| Chain 2: Fc-Linker IFN | SNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCSV |
| | MHEALHNHYTQKSLSLSLGKGGGGSGGGGSGGGGSCDLPQTHSLGSRRTLML |
| | LAQMRRISLFSCLKDRHDFGFPQEEFGNQFQKAETIPVLHEMIQQIFNLFST |
| | KDSSAAWDETLLDKFYTELYQQLNDLEACVIQGVGVTETPLMKEDSILAVRK |
| | YFQRITLYLKEKKYSPCAWEVVRAEIMRSFSLSTNLQESLRSKE (SEQ ID |
| | NO: 129) |
| | Chain 2: |
| | ESKYGPPCPPCPAPPVAGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCSV |
| | MHEALHNHYTQKSLSLSLGKGGGGSGGGGSGGGGSCDLPQTHSLGSRRTLML |
| | LAQMRRISLFSCLKDRHDFGFPQEEFGNQFQKAETIPVLHEMIQQIFNLFST |
| | KDSSAAWDETLLDKFYTELYQQLNDLEACVIQGVGVTETPLMKEDSILAVRK |
| | YFQRITLYLKEKKYSPCAWEVVRAEIMRSFSLSTNLQESLRSKE (SEQ ID |
| | NO: 129) |
| Fc-R1-IFN (homodimeric, | Chain 1: |
| both chains identical) | ESKYGPPCPPCPAPPVAGPSVFLFPPKPKDTLMISRTPEVTCVVVDVSQEDP |
| Chain 1: Fc-linker- | EVQFNWYVDGVEVHNAKTKPREEQFNSTYRVVSVLTVLHQDWLNGKEYKCKV |
| IFNAR1-linker-IFN | SNKGLPSSIEKTISKAKGQPREPQVYTLPPSQEEMTKNQVSLTCLVKGFYPS |
| Chain 2: Fc-linker- | DIAVEWESNGQPENNYKTTPPVLDSDGSFFLYSRLTVDKSRWQEGNVFSCSV |
| IFNAR1-linker-IFN | MHEALHNHYTQKSLSLSLGKGGGGSGGGGSGGGGSKNLKSPQKVEVDIIDDN |
| | FILRWNRSDESVGNVTFSFDYQKTGMDNWIKLSGCQNITSTKCNFSSLKLNV |
| | YEEIKLRIRAEKENTSSWYEVDSFTPFRKAQIGPPEVHLEAEDKAIVIHISP |
| | GTKDSVMWALDGLSFTYSLVIWKNSSGVEERIENIYSRHKIYKLSPETTYCL |
| | KVKAALLTSWKIGVYSPVHCIKTTVENELPPPENIEVSVQNQNYVLKWDYTY |
| | ANMTFQVQWLHAFLKRNPGNHLYKWKQIPDCENVKTTQCVFPQNVFQKGIYL |
| | LRVQASDGNNTSFWSEEIKFDTEIQGGGGSCDLPQTHSLGSRRTLMLLAQMR |
| | RISLFSCLKDRHDFGFPQEEFGNQFQKAETIPVLHEMIQQIFNLFSTKDSSA |
| | AWDETLLDKFYTELYQQLNDLEACVIQGVGVTETPLMKEDSILAVRKYFQRI |
| | TLYLKEKKYSPCAWEVVRAEIMRSFSLSTNLQESLRSKE (SEQ ID |
| | NO: 130) |
| | Chain 2: |
| | ESKYGPPCPPCPAPPVAGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCSV |
| | MHEALHNHYTQKSLSLSLGKGGGGSGGGGSGGGGSKNLKSPQKVEVDIIDDN |
| | FILRWNRSDESVGNVTFSFDYQKTGMDNWIKLSGCQNITSTKCNFSSLKLNV |
| | YEEIKLRIRAEKENTSSWYEVDSFTPFRKAQIGPPEVHLEAEDKAIVIHISP |
| | GTKDSVMWALDGLSFTYSLVIWKNSSGVEERIENIYSRHKIYKLSPETTYCL |
| | KVKAALLTSWKIGVYSPVHCIKTTVENELPPPENIEVSVQNQNYVLKWDYTY |
| | ANMTFQVQWLHAFLKRNPGNHLYKWKQIPDCENVKTTQCVFPQNVFQKGIYL |
| | LRVQASDGNNTSFWSEEIKFDTEIQGGGGSCDLPQTHSLGSRRTLMLLAQMR |
| | RISLFSCLKDRHDFGFPQEEFGNQFQKAETIPVLHEMIQQIFNLFSTKDSSA |
| | AWDETLLDKFYTELYQQLNDLEACVIQGVGVTETPLMKEDSILAVRKYFQRI |
| | TLYLKEKKYSPCAWEVVRAEIMRSFSLSTNLQESLRSKE (SEQ ID |
| | NO: 130) |
| Fc-IFN x Fc | Chain 1: |
| (heterodimeric) | ESKYGPPCPPCPAPPVAGPSVFLFPPKPKDTLMISRTPEVTCVVVDVSQEDP |
| Chain 1: FC_{knob}-linker-IFN | EVQFNWYVDGVEVHNAKTKPREEQFNSTYRVVSVLTVLHQDWLNGKEYKCKV |
| Chain 2: FCₕₒₗₑ | SNKGLPSSIEKTISKAKGQPREPQVYTLPPSQEEMTKNQVSLWCLVKGFYPS |
| | DIAVEWESNGQPENNYKTTPPVLDSDGSFFLYSRLTVDKSRWQEGNVFSCSV |
| | MHEALHNHYTQKSLSLSLGKGGGGSGGGGSGGGGSCDLPQTHSLGSRRTLML |
| | LAQMRRISLFSCLKDRHDFGFPQEEFGNQFQKAETIPVLHEMIQQIFNLFST |
| | KDSSAAWDETLLDKFYTELYQQLNDLEACVIQGVGVTETPLMKEDSILAVRK |
| | YFQRITLYLKEKKYSPCAWEVVRAEIMRSFSLSTNLQESLRSKE (SEQ ID |
| | NO: 131) |
| | Chain 2: |
| | ESKYGPPCPPCPAPPVAGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLSCAVKGFYPS |
| | DIAVEWESNGQPENNYKTTPPVLDSDGSFFLVSRLTVDKSRWQEGNVFSCSV |
| | MHEALHNRFTQKSLSLSPGK (SEQ ID NO: 132) |
| Fc-IFN x Fc-R1 | Chain 1: |
| (heterodimeric) | ESKYGPPCPPCPAPPVAGPSVFLFPPKPKDTLMISRTPEVTCVVVDVSQEDP |
| Chain 1: FC_{knob}-linker-IFN | EVQFNWYVDGVEVHNAKTKPREEQFNSTYRVVSVLTVLHQDWLNGKEYKCKV |
| Chain 2: FCₕₒₗₑ-linker- | SNKGLPSSIEKTISKAKGQPREPQVYTLPPSQEEMTKNQVSLWCLVKGFYPS |
| IFNAR1 | DIAVEWESNGQPENNYKTTPPVLDSDGSFFLYSRLTVDKSRWQEGNVFSCSV |
| | MHEALHNHYTQKSLSLSLGKGGGGSGGGGSGGGGSCDLPQTHSLGSRRTLML |
| | LAQMRRISLFSCLKDRHDFGFPQEEFGNQFQKAETIPVLHEMIQQIFNLFST |
| | KDSSAAWDETLLDKFYTELYQQLNDLEACVIQGVGVTETPLMKEDSILAVRK |
| | YFQRITLYLKEKKYSPCAWEVVRAEIMRSFSLSTNLQESLRSKE (SEQ ID |
| | NO: 131) |
| | Chain 2: |
| | ESKYGPPCPPCPAPPVAGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLSCAVKGFYPS |
| | DIAVEWESNGQPENNYKTTPPVLDSDGSFFLVSRLTVDKSRWQEGNVFSCSV |
| | MHEALHNRFTQKSLSLSPGKGGGGSGGGGSGGGGSKNLKSPQKVEVDIIDDN |
| | FILRWNRSDESVGNVTFSFDYQKTGMDNWIKLSGCQNITSTKCNFSSLKLNV |
| | YEEIKLRIRAEKENTSSWYEVDSFTPFRKAQIGPPEVHLEAEDKAIVIHISP |
| | GTKDSVMWALDGLSFTYSLVIWKNSSGVEERIENIYSRHKIYKLSPETTYCL |
| | KVKAALLTSWKIGVYSPVHCIKTTVENELPPPENIEVSVQNQNYVLKWDYTY |
| | ANMTFQVQWLHAFLKRNPGNHLYKWKQIPDCENVKTTQCVFPQNVFQKGIYL |
| | LRVQASDGNNTSFWSEEIKFDTEIQ (SEQ ID NO: 133) |
| Fc-R1-IFN x Fc | Chain 1: |
| (heterodimeric) | ESKYGPPCPPCPAPPVAGPSVFLFPPKPKDTLMISRTPEVTCVVVDVSQEDP |
| Chain 1: FC_{knob}-linker- | EVQFNWYVDGVEVHNAKTKPREEQFNSTYRVVSVLTVLHQDWLNGKEYKCKV |
| IFNAR1-linker-IFN | SNKGLPSSIEKTISKAKGQPREPQVYTLPPSQEEMTKNQVSLWCLVKGFYPS |
| Chain 2: FCₕₒₗₑ | DIAVEWESNGQPENNYKTTPPVLDSDGSFFLYSRLTVDKSRWQEGNVFSCSV |
| | MHEALHNHYTQKSLSLSLGKGGGGSGGGGSGGGGSKNLKSPQKVEVDIIDDN |
| | FILRWNRSDESVGNVTFSFDYQKTGMDNWIKLSGCQNITSTKCNFSSLKLNV |
| | YEEIKLRIRAEKENTSSWYEVDSFTPFRKAQIGPPEVHLEAEDKAIVIHISP |
| | GTKDSVMWALDGLSFTYSLVIWKNSSGVEERIENIYSRHKIYKLSPETTYCL |
| | KVKAALLTSWKIGVYSPVHCIKTTVENELPPPENIEVSVQNQNYVLKWDYTY |
| | ANMTFQVQWLHAFLKRNPGNHLYKWKQIPDCENVKTTQCVFPQNVFQKGIYL |
| | LRVQASDGNNTSFWSEEIKFDTEIQGGGGSCDLPQTHSLGSRRTLMLLAQMR |
| | RISLFSCLKDRHDFGFPQEEFGNQFQKAETIPVLHEMIQQIFNLFSTKDSSA |
| | AWDETLLDKFYTELYQQLNDLEACVIQGVGVTETPLMKEDSILAVRKYFQRI |
| | TLYLKEKKYSPCAWEVVRAEIMRSFSLSTNLQESLRSKE (SEQ ID |
| | NO: 134) |
| | Chain 2: |
| | ESKYGPPCPPCPAPPVAGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLSCAVKGFYPS |
| | DIAVEWESNGQPENNYKTTPPVLDSDGSFFLVSRLTVDKSRWQEGNVFSCSV |
| | MHEALHNRFTQKSLSLSPGK (SEQ ID NO: 132) |
| Fc x Fc-R1 | Chain 1: |
| (heterodimeric) | ESKYGPPCPPCPAPPVAGPSVFLFPPKPKDTLMISRTPEVTCVVVDVSQEDP |
| Chain 1: FC_{knob} | EVQFNWYVDGVEVHNAKTKPREEQFNSTYRVVSVLTVLHQDWLNGKEYKCKV |
| Chain 2: FCₕₒₗₑ(with star | SNKGLPSSIEKTISKAKGQPREPQVYTLPPSQEEMTKNQVSLWCLVKGFYPS |
| mutations)-linker-IFNAR1 | DIAVEWESNGQPENNYKTTPPVLDSDGSFFLYSRLTVDKSRWQEGNVFSCSV |
| | MHEALHNHYTQKSLSLSLGK (SEQ ID NO: 135) |
| | Chain 2: |
| | ESKYGPPCPPCPAPPVAGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLSCAVKGFYPS |
| | DIAVEWESNGQPENNYKTTPPVLDSDGSFFLVSRLTVDKSRWQEGNVFSCSV |
| | MHEALHNRFTQKSLSLSPGKGGGGSGGGGSGGGGSKNLKSPQKVEVDIIDDN |
| | FILRWNRSDESVGNVTFSFDYQKTGMDNWIKLSGCQNITSTKCNFSSLKLNV |
| | YEEIKLRIRAEKENTSSWYEVDSFTPFRKAQIGPPEVHLEAEDKAIVIHISP |
| | GTKDSVMWALDGLSFTYSLVIWKNSSGVEERIENIYSRHKIYKLSPETTYCL |
| | KVKAALLTSWKIGVYSPVHCIKTTVENELPPPENIEVSVQNQNYVLKWDYTY |
| | ANMTFQVQWLHAFLKRNPGNHLYKWKQIPDCENVKTTQCVFPQNVFQKGIYL |
| | LRVQASDGNNTSFWSEEIKEDTEIQ (SEQ ID NO: 136) |

### 9.1.2. Production of IFN Receptor Antagonist Constructs

Constructs encoding IFN receptor antagonists were generated in standard mammalian protein expression DNA vectors (pcDNA3.4 or similar) suitable for high yield protein production and containing standard elements such as promoter sequence, polyA sequence, regulatory elements, and resistance genes. Where applicable, sequences were codon optimized. A 29-amino acid signal sequence from murine inactive tyrosine-protein kinase transmembrane receptor ROR1 (mROR1) was added to the N-termini of the constructs to serve as a signal for secretion. All IFN receptor antagonists were expressed as preproteins containing the signal sequence which is cleaved by intracellular processing to produce a mature protein. The constructs were expressed in Expi293FTM cells by transient transfection (Thermo Fisher Scientific). Proteins in Expi293F supernatant were purified using the ProteinMaker system (Protein BioSolutions, Gaithersburg, MD) with either HiTrap™ Protein G HP or MabSelect SuRe pcc columns (Cytiva). After single step elution, the proteins were neutralized, dialyzed into a final buffer of phosphate buffered saline (PBS) with 5% glycerol, aliquoted and stored at −80° C. Samples were further analyzed by SE-UPLC to determine the presence of high or low molecular weight species relative to the species of interest.

### 9.1.3. Engineering of KG-1a Reporter Cells

The promyeloblast macrophage cell line KG-1a was transduced with an Interferon-Stimulated Response Element (ISRE)-driven luciferase reporter construct and maintained in Iscove's modified Dulbecco's medium supplemented with 2 mM L-Glutamine/Penicillin/Streptomycin+20% FBS+1 g/mL puromycin. A single cell clone, KG-1a/ISRE-Luc with high responsiveness to IFNα2b was isolated. PDL1 was knocked out in this clone using CRISPR-Cas9 technology, and the resulting cell line, KG-1a/ISRE-Luc/PDL1 KO, was validated by flow cytometry. KG-1a/ISRE-Luc were engineered to overexpress PDL1 (amino acids M1-T290 of accession #NP_054862.1), and the resulting cell line, KG-1a/ISRE-Luc/hPDL1 was validated by flow cytometry.

### 9.1.4. Staining and Flow Cytometry

KG-1a/ISRE-Luc cells were plated at a density of 2.5×10⁵ per well and incubated with a serial dilution of IFN receptor antagonists for 30 minutes on ice. The cells were washed twice and counterstained with goat anti-human IgG F(ab′)₂ AF647 for another 30 minutes on ice. Upon completion of the staining, cells were washed and fixed, and data was acquired on iQue Plus (Sartorius).

### 9.1.5. Luciferase Assay Setup for IFN Signaling Assessment

RPM11640 media supplemented with 2 mM L-Glutamine/Penicillin/Streptomycin+10% FBS was used as assay medium to prepare cell suspensions and dilutions of IFN receptor antagonists or control constructs.

The day of the assay, KG-1a/ISRE-Luc reporter cells were centrifuged and resuspended in assay medium at a density of 5×10⁵/mL. IFNα2b, IFN receptor antagonists, and control constructs were diluted 1:5 following a 11-point dilution range, with the 12^{th} point containing no recombinant protein. 2.5×10⁴ reporter cells were added to 96-well white flat bottom plates and incubated with serially diluted IFNα2b, IFN receptor antagonists, and control constructs in the presence or absence of 200 pM IFNα2b, 45 pM IFNα2b, or 90 pM IFNβ. Plates were incubated for 5 hours at 37° C. and 5% CO₂ before the addition of 100 μL ONE-Glo™ (Promega) reagent to lyse cells and detect luciferase activity. The emitted light was captured in relative light units (RLU) on a multilabel plate reader Envision (PerkinElmer). All serial dilutions were tested in duplicates.

### 9.1.6. Cell Viability Assessment Via Luminescence Assay

Iscove's modified Dulbecco's media supplemented with 2 mM L-Glutamine/Penicillin/Streptomycin+20% Fetal Bovine Serum (FBS) was used as the assay medium to prepare cell suspensions and dilutions of recombinant IFNα2b, IFNβ, or control constructs.

The day of the assay, KG-1a/ISRE-Luc reporter cells were centrifuged, resuspended in assay medium at a density of 1×10⁵/mL, plated at 5×10³ reporter cells/well in 96-well white flat bottom plates, and incubated with either a titration of recombinant IFNα2b, IFNβ, or control constructs in combination with a constant amount of IFNα2b (900 pM) or IFNβ (30 pM). All constructs were serially diluted (1:5) over a 9-point titration range (50 nM to 5.12 fM) and a 10th point containing no recombinant protein, or constant IFNα2b or IFNβ. Plates were incubated for 5 days at 37° C. and 5% CO₂. On the 5^{th} day, 100 mL of RealTime-Glo™ (Promega) reagent was added to each well and incubated for 1 h at 37° C. and 5% CO₂, followed by detection of NanoLuc® luciferase activity. The emitted light was measured in relative light units (RLU) on a multilabel plate reader Envision (PerkinElmer). EC₅₀ values of the tested constructs were determined using GraphPad Prism™ software from a four-parameter logistic equation over a 10-point dose-response curve. Percent inhibition of cytotoxicity response for each tested construct was calculated using the formula: % inhibition=((max luminescence (construct)−min luminescence (construct))/(max IFN luminescence−min IFN luminescence))×100. All serial dilutions were tested in duplicates.

### 9.2. Example 1: In Vitro Binding Affinities of IFN Receptor Antagonists

Targeted monovalent and bivalent IFN receptor antagonist and control constructs were designed and produced as described in Sections 9.1.1 and 9.1.2. The binding affinities of IFN receptor antagonists and control constructs were assessed as described in Section 9.1.4, whereby data obtained from viable cells were used as a measure of binding affinity. The strongest binding affinity was observed with cis-masked bivalent Fc-R1-IFNα2b and monovalent Fc-R1-IFNα2b constructs (FIG. 2A).

The activity of IFN receptor antagonists and control constructs was evaluated in KG-1a/ISRE-Luc reporter cells as described in Section 9.1.4. The potency of both monovalent and bivalent Fc-IFNα2b was reduced compared to IFNα2b. Compared to Fc-IFNα2b, cis-masked bivalent Fc-R1-IFNα2b (bivalent Fc-R1-IFNα2b) and monovalent Fc-R1-IFNα2b (Fc-R1-IFNα2b), and to a lower extent trans-masked monovalent Fc-IFNα2b×R1 (Fc-IFNα2b×R1) constructs have reduced potency.

Collectively, these results indicate that cis-masked constructs comprising IFNAR1 masking moieties occupy IFNAR2 receptors on the cell membrane and prevent free IFN binding to these receptors, attenuating downstream intracellular cascades.

### 9.3. Example 2: In Vitro Signaling Activity of Targeted IFN Receptor Antagonists

Targeted monovalent and bivalent IFN receptor antagonist and control constructs were designed and produced as described in Sections 9.1.1 and 9.1.2. The signaling activity of targeted IFN receptor antagonist and control constructs was assessed using KG-1a/ISRE-Luc reporter cells as described in Sections 9.1.3 and 9.1.5.

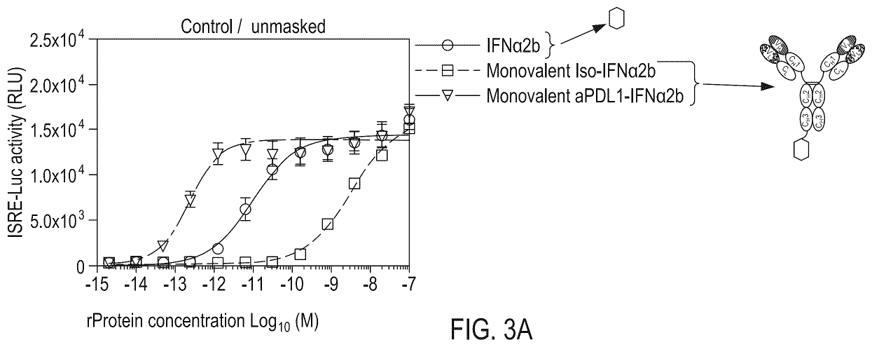

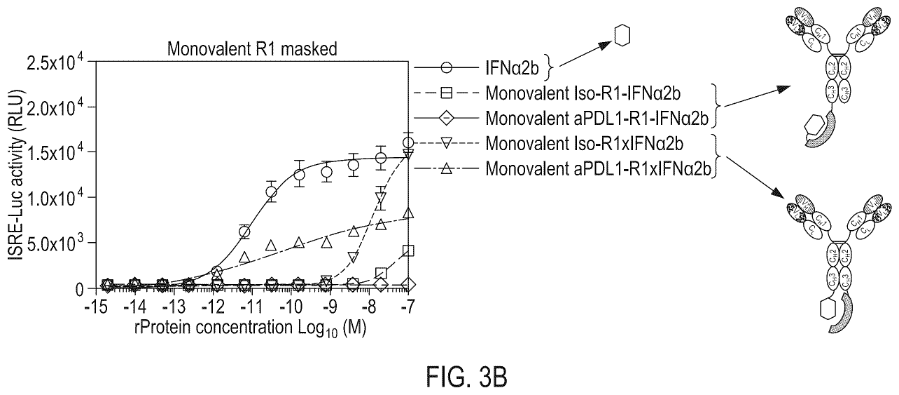

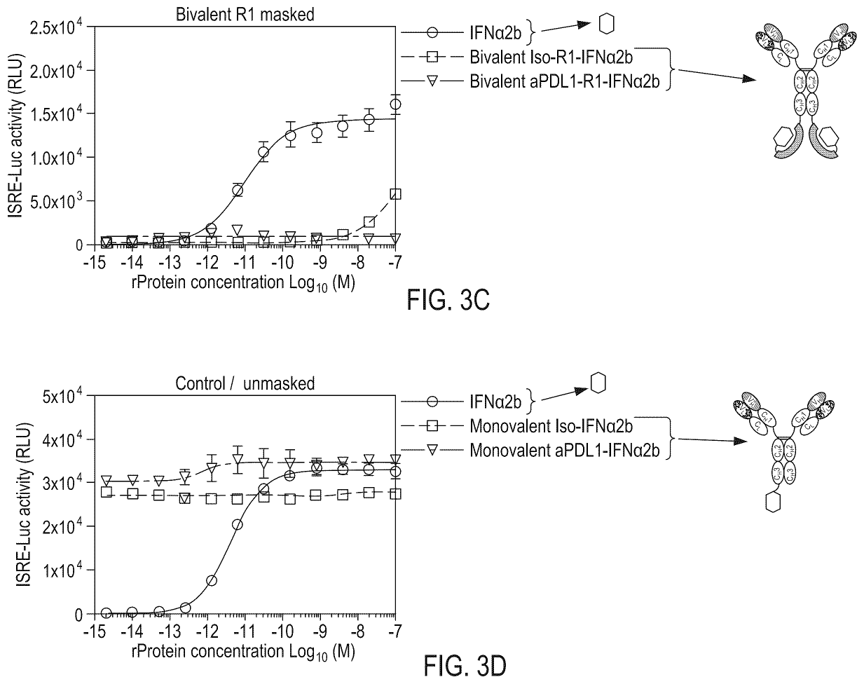

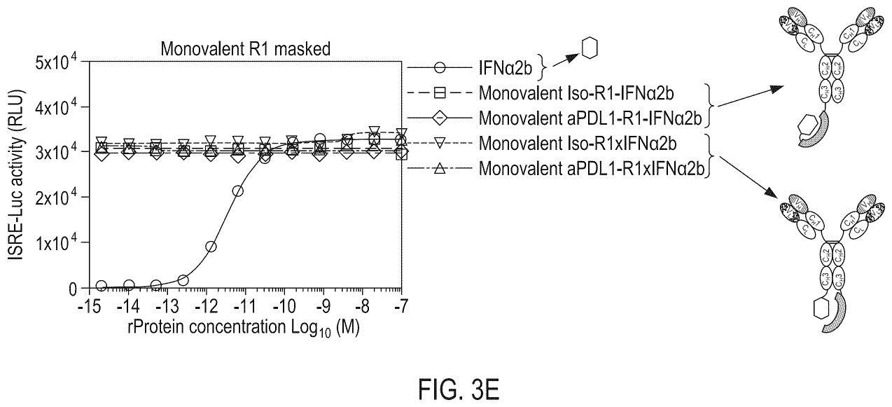

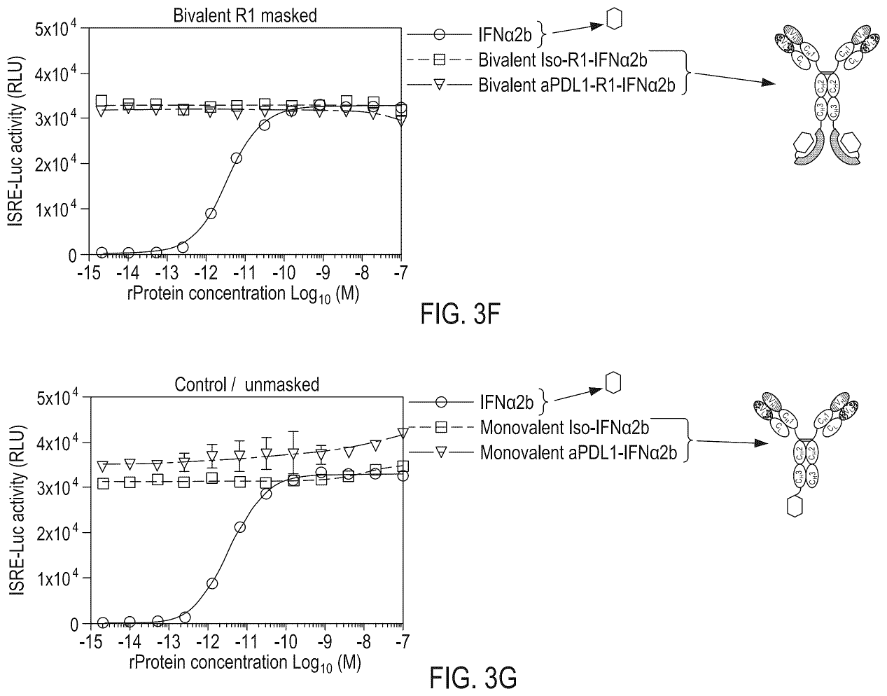

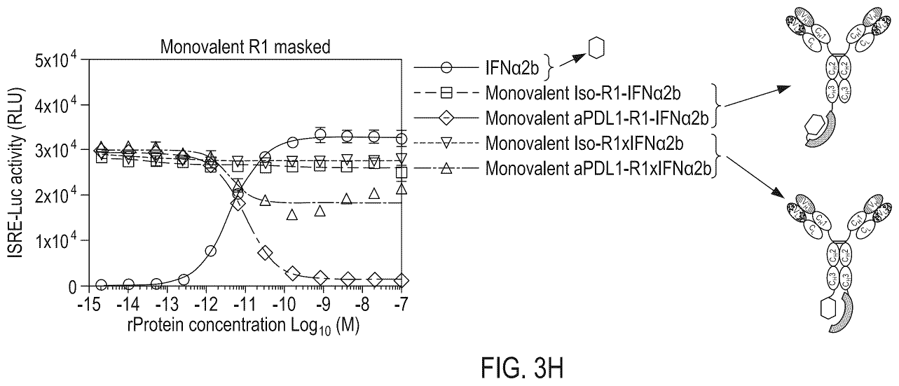

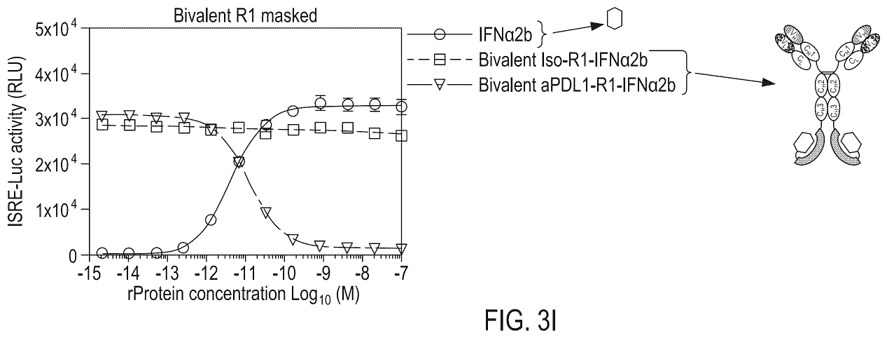

In the first assessment, the activity of PDL1-targeted monovalent IFN receptor antagonists and isotype control constructs was evaluated in PDL1 overexpressing (OE) KG-1a/ISRE-Luc reporter cells in the absence of IFNα2b in the buffer (FIGS. 3A-3C). In comparison to IFNα2b, non-targeted Iso-IFNα2b construct had lower potency (FIG. 3A). In comparison to the non-targeted Ab-IFNα2b, the masked non-targeted constructs had lower potency, regardless of their masking moieties, with the monovalent cis-masked (monovalent Iso-R1-IFNα2b) and bivalent masked (bivalent Iso-R1-IFNα2b) having the lowest potency (FIGS. 3B and 3C). The construct aPDL1-IFNα2b had the strongest activity surpassing the activity displayed by the IFNα2b control (FIG. 3A), underscoring the effect of PDL1 targeting. The other targeting-dependent activation of IFNAR was observed with trans-masked IFNAR1 masking moiety construct, wherein the activity of monovalent aPDL1-R1×IFNα2b was more potent than that of Iso-R1×IFNα2b. No targeting-dependent increases in activity were observed with the cis-masked monovalent and bivalent R1-masked constructs.

Next, the activity of PDL1-targeted IFN receptor antagonists and isotype control constructs were evaluated in the presence of 200 pM IFNα2b in PDL1 KO cells (FIGS. 3D-F). All PDL1-targeted monovalent and bivalent IFN receptor antagonists and isotype control constructs were associated with relatively constant levels of reporter activity, attributable to the presence of 200 pM IFNα2b, and none of the constructs displayed any concentration-dependent changes in activity, demonstrating the IFN receptor antagonists do not have target independent antagonistic activity.

In the next set of assessments, the activity of the PDL1 targeted IFN receptor antagonists and isotype control constructs were evaluated in the presence of 200 pM IFNα2b in PDL1 overexpressing (OE) cells (FIGS. 3G-3I). Titration with IFNα2b resulted in comparable concentration-response curves (FIGS. 3D and 3F). When the PDL1-targeted monovalent IFN receptor antagonists and isotype control constructs were evaluated using PDL1 OE cells, PDL1-targeted construct with IFNAR1 masking moiety, aPDL1-R1-IFNα2b, demonstrated a concentration-dependent reduction in activity, indicative of a targeting-dependent IFNAR blockade (FIG. 3H). Furthermore, the structure of aPDL1-R1-IFNα2b seemed to be associated with its strong reduction in activity. The masking moiety and the IFN moiety of aPDL1-R1-IFNα2b are on the same polypeptide chain and connected with a linker as depicted in FIG. 1B. A similar but trans-masked monovalent construct, a PDL1-IFNα2b×R1, wherein the masking moiety and the IFN moiety are on different polypeptide chains as depicted in FIG. 1D, was associated with an incomplete IFNAR blockade.

The bivalent construct aPDL1-R1-IFNα2b was associated with a similar reduction in activity as the monovalent cis-masked construct (FIG. 3I). Both the monovalent aPDL1-R1-IFNα2b and bivalent aPDL1-R1-IFNα2b achieved a complete blocking of IFNAR activity, and the trans-masked aPDL1-IFNα2b×PDL1-R1 achieved a partial blocking of IFNAR activity (FIGS. 3H and 3I).

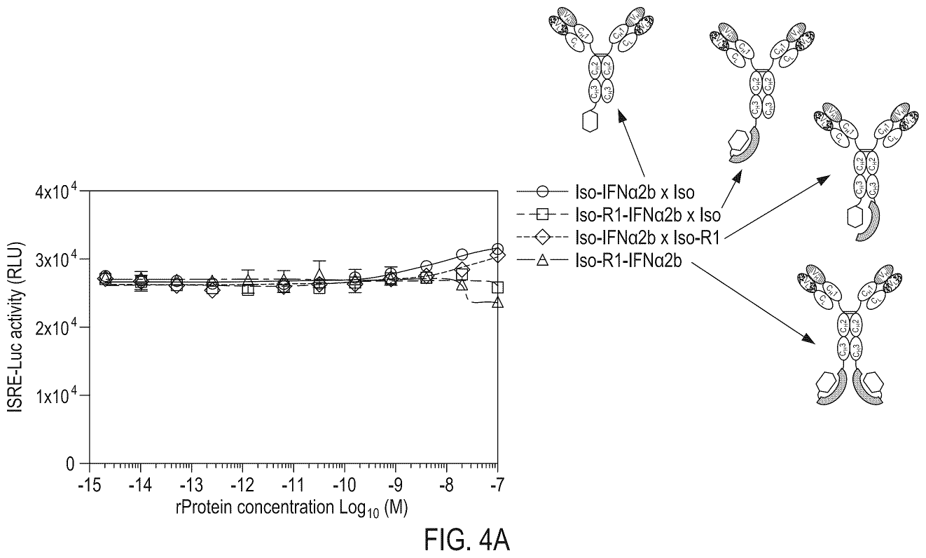

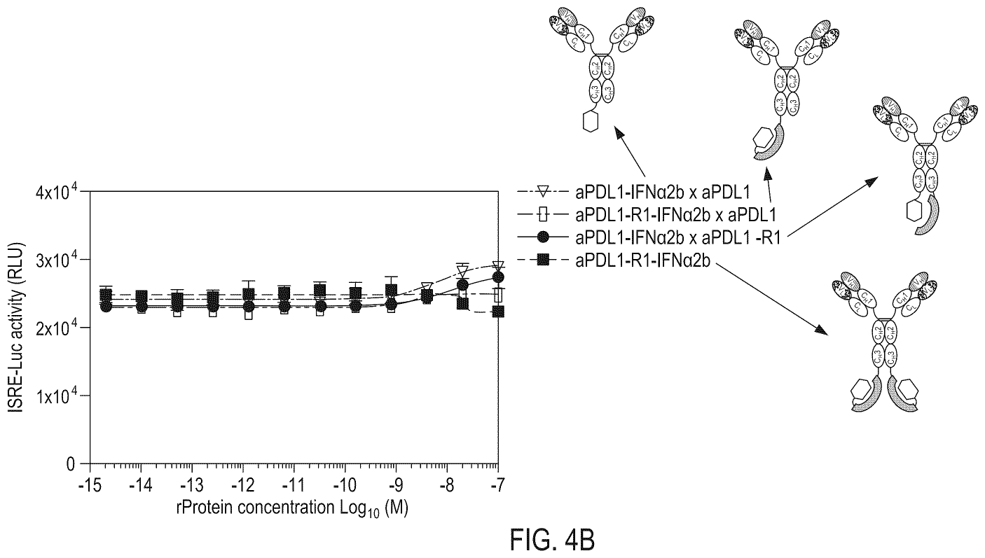

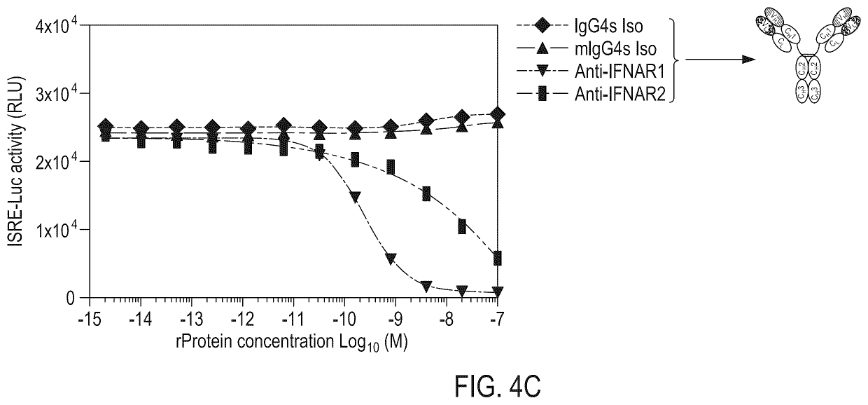

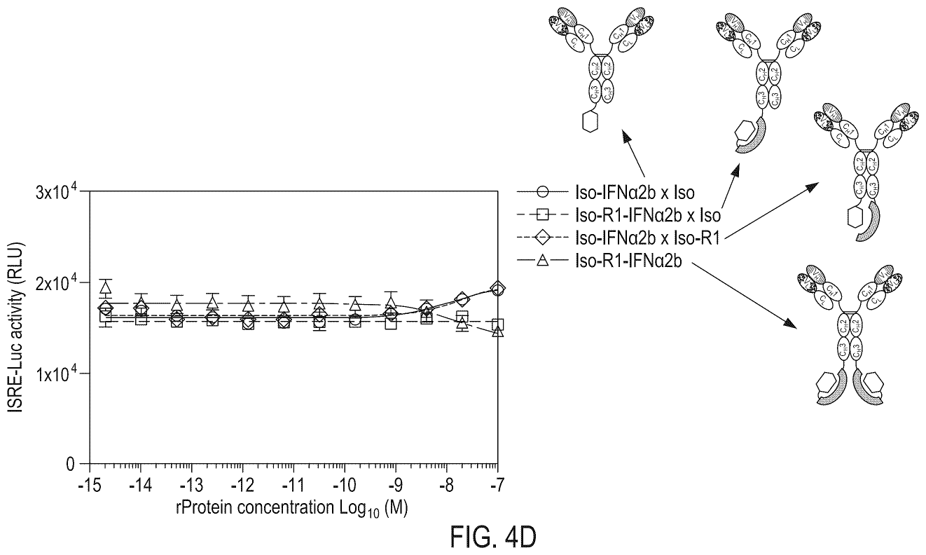

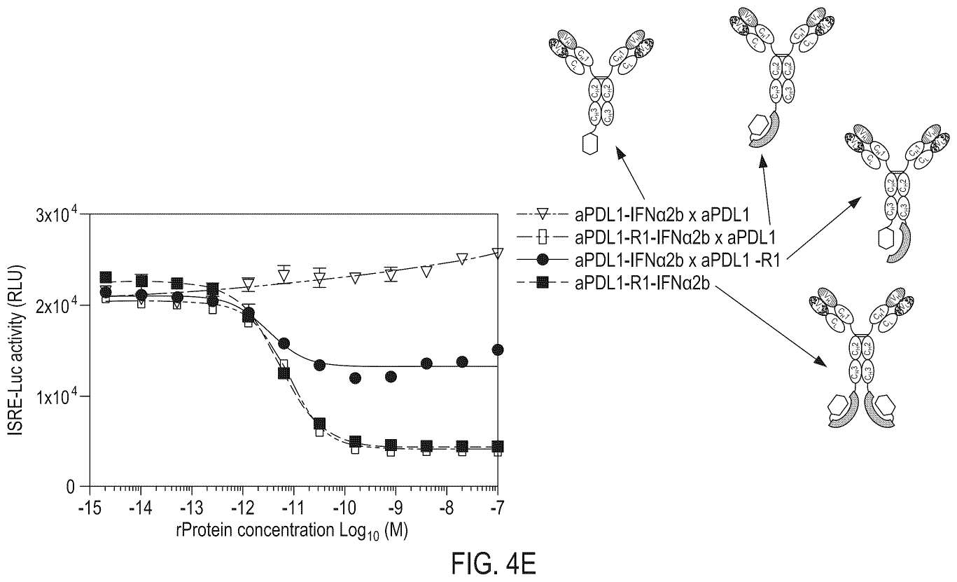

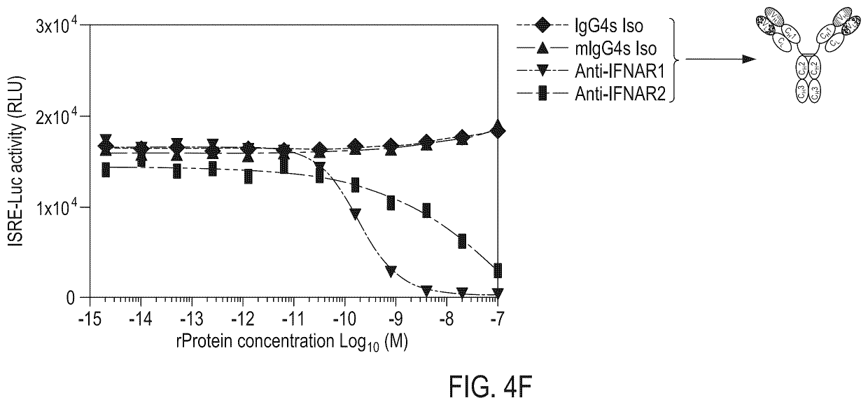

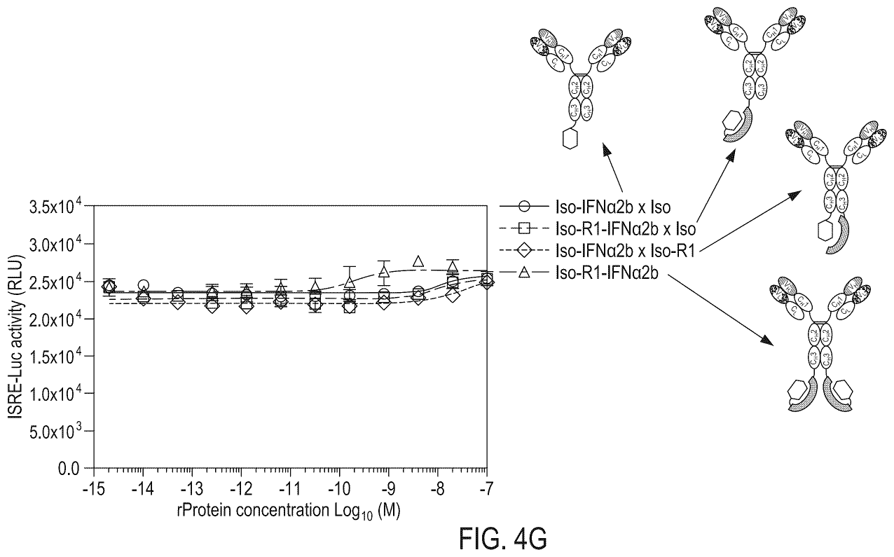

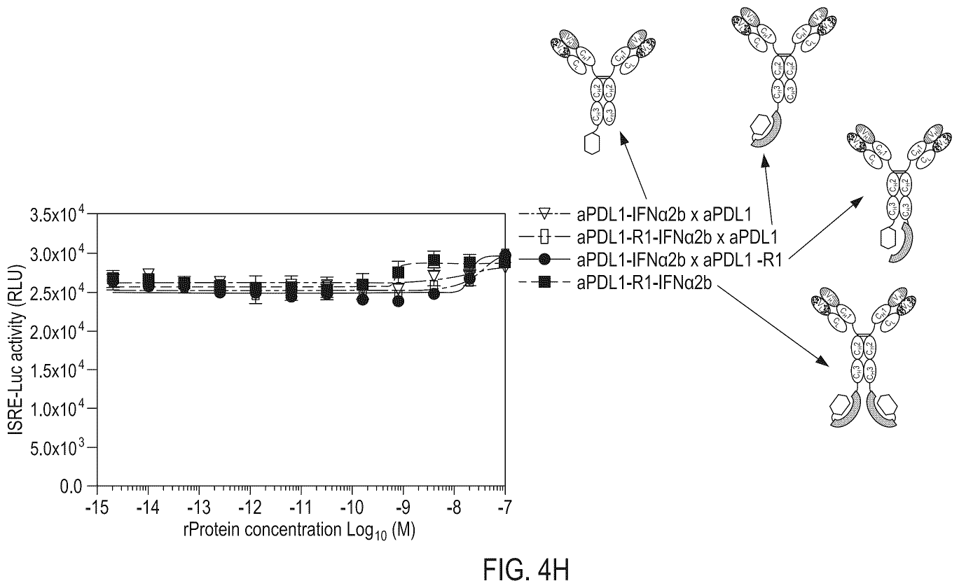

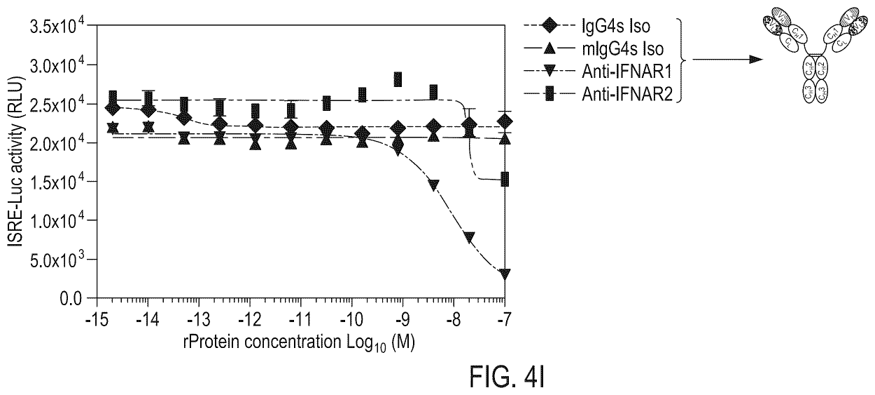

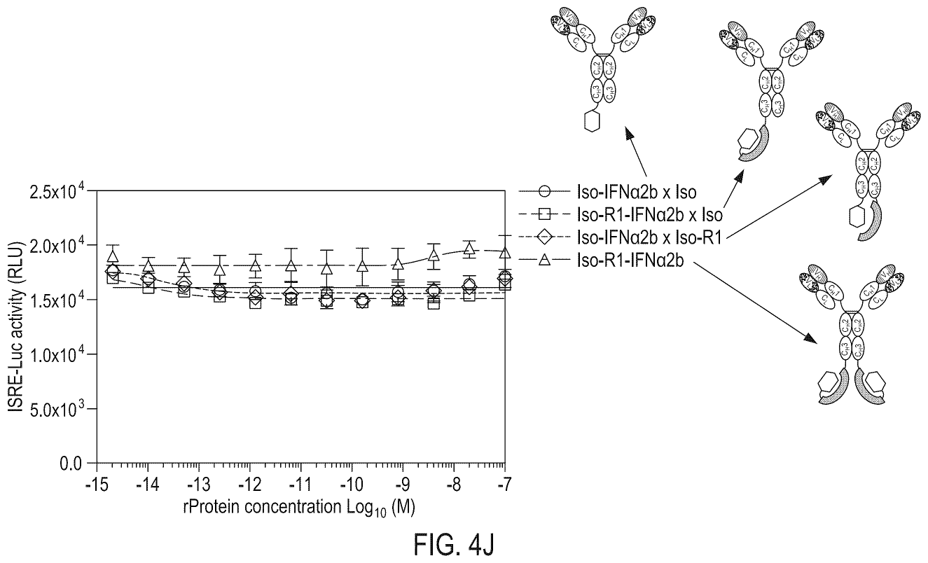

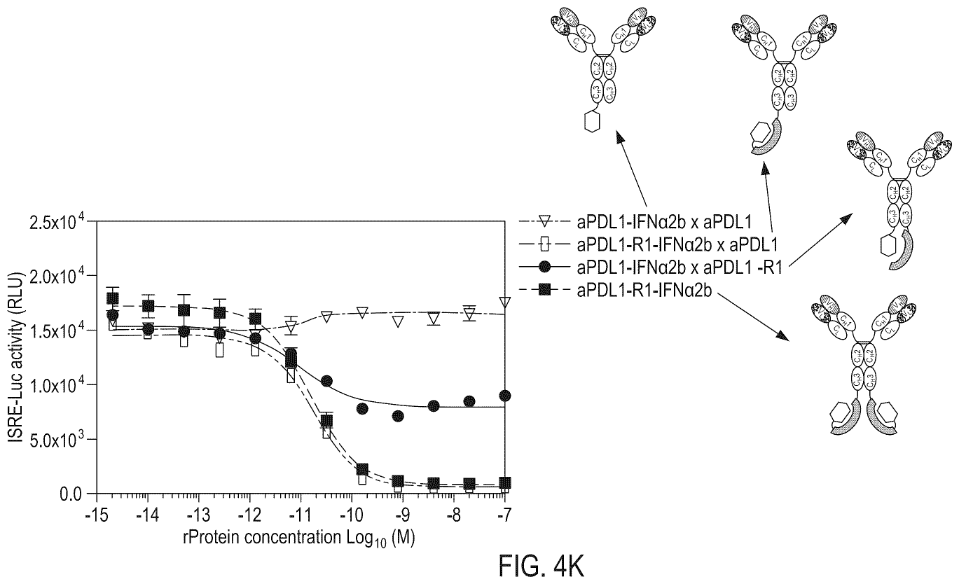

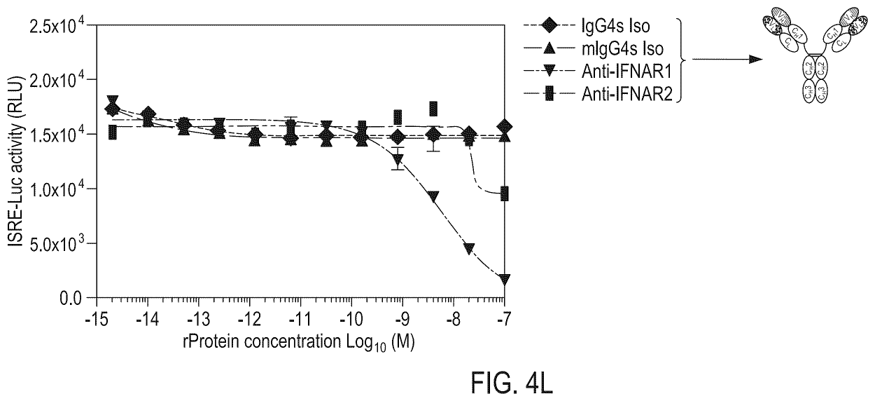

Next, the activity of the PDL1 targeted IFN receptor antagonists and isotype control constructs were evaluated in comparison with exemplary anti-IFNAR1 and anti-IFNAR2 antibodies in the presence of 45 pM IFNα2b (FIGS. 4A-4F) or 95 pM IFNβ (FIGS. 4G-4L) in PDL1 KO (FIGS. 4A-4C and 4G-4I) or OE cells (FIGS. 4D-4F and 4J-4L). Titration with masked monovalent or bivalent PDL1-R1-IFN resulted in dose-dependent PDL1 dependent inhibition-response curves against IFNα2b (FIGS. 4A-4F) and IFNβ (FIGS. 4G-4L). In contrast, anti-IFNAR1 and IFNAR2 antibodies yielded lower potency of blockade against either IFNα2b or IFNβ, which was not PDL1-dependent. Altogether, these data demonstrate the Ab-R1-IFNα2b monovalent and bivalent masked format enable potent targeted IFN receptor blockade.

### 9.4. Example 3: In Vitro Cytoprotective Activity of Targeted IFN Receptor Antagonists

Targeted monovalent and bivalent IFN receptor antagonist and control constructs were designed and produced as described in Sections 9.1.1 and 9.1.2. The cytoprotective activity of targeted IFN receptor antagonist and control constructs was assessed using KG-1a/ISRE-Luc reporter cells as described in Sections 9.1.3 and 9.1.6.

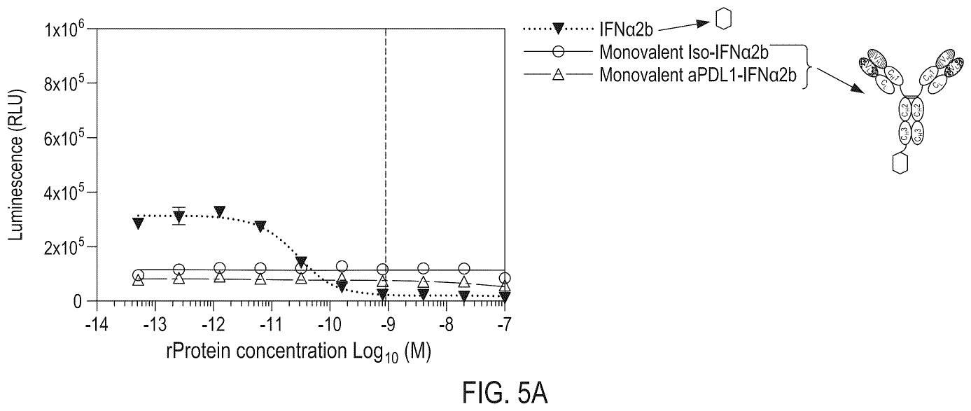

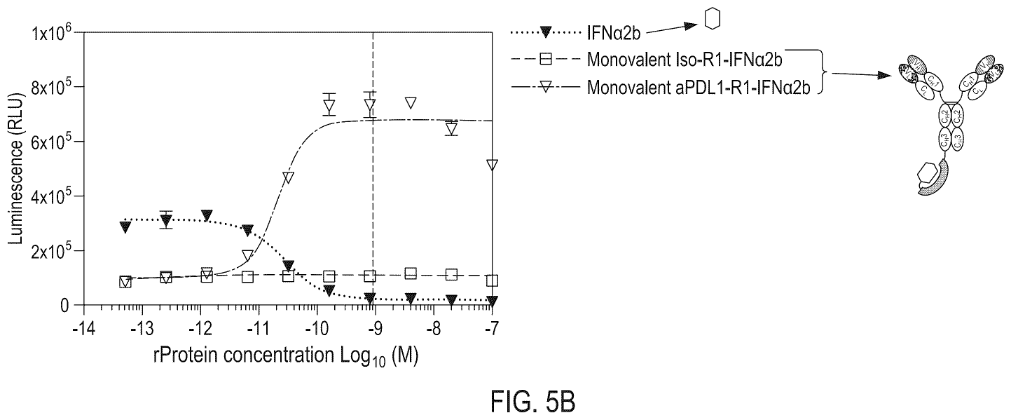

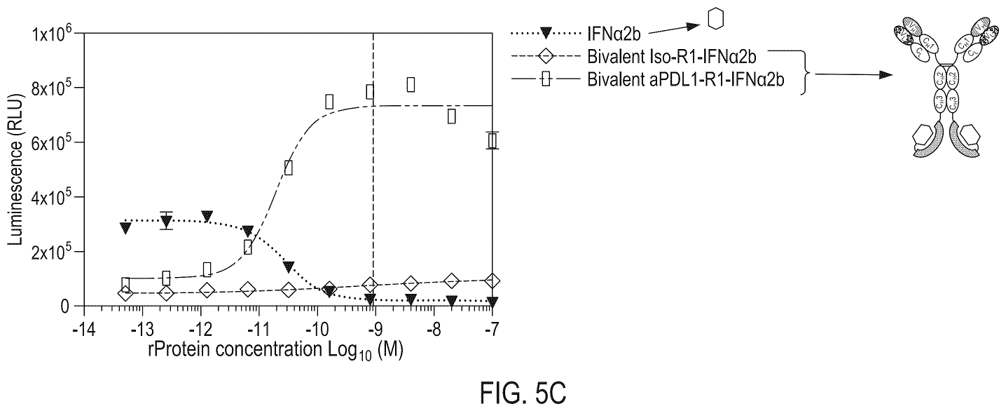

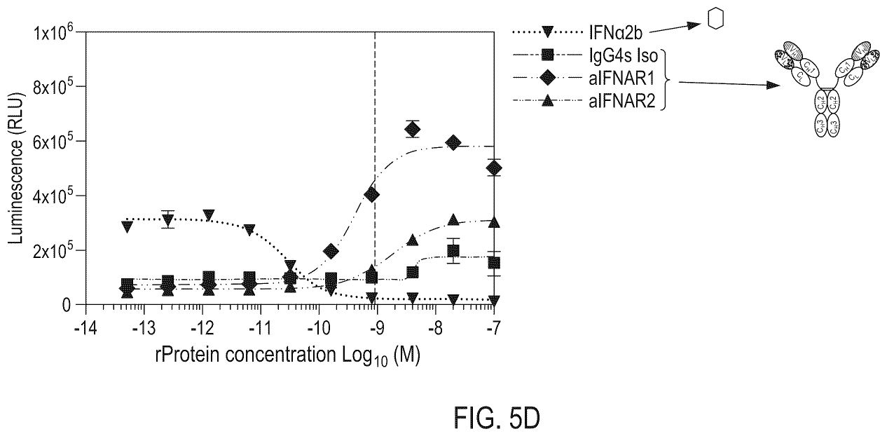

In the first set of assessments, the cytoprotective activity of the IFN receptor antagonist and control constructs were evaluated in PDL1 overexpressing (OE) and PDL1 KO KG-1a/ISRE-Luc reporter cells that were co-treated with 900 pM IFNα2b for five days. This IFNα2b treatment resulted either in cytostatic response, detected as a flat luminescence curve due to lack of cell division, or cytotoxicity, detected as a decrease in luminescence signal due to reduction of cell numbers. Neither PDL1 targeted unmasked nor isotype control unmasked constructs blocked cytostatic/cytotoxic responses to IFNα2b treatment (FIGS. 5A and 6A, and Table E-2). In contrast, PDL1 targeted monovalent and bivalent masked IFN receptor antagonist were associated with concentration-dependent increases in luminescence in PDL1 OE KG-1a/ISRE-Luc reporter cells, whereas isotype controls were associated with relatively constant levels of luminescence (FIGS. 5B-5C) and percent inhibition of the IFNα2b treatment-induced cytostatic/cytotoxic response (Table E-2). In PDL1 KO KG-1a/ISRE-Luc reporter cells, none of the monovalent and bivalent IFN receptor antagonists were associated with increases in luminescence (FIGS. 6B-6C) or percent inhibition (Table E-2), suggesting that the cytoprotective activity of IFN receptor antagonists are target-dependent. Anti-IFNAR1 and anti-IFNAR2 antibodies yielded relatively modest cytoprotective activity, which was not PDL1-dependent (FIGS. 5D and 6D, and Table E-2).

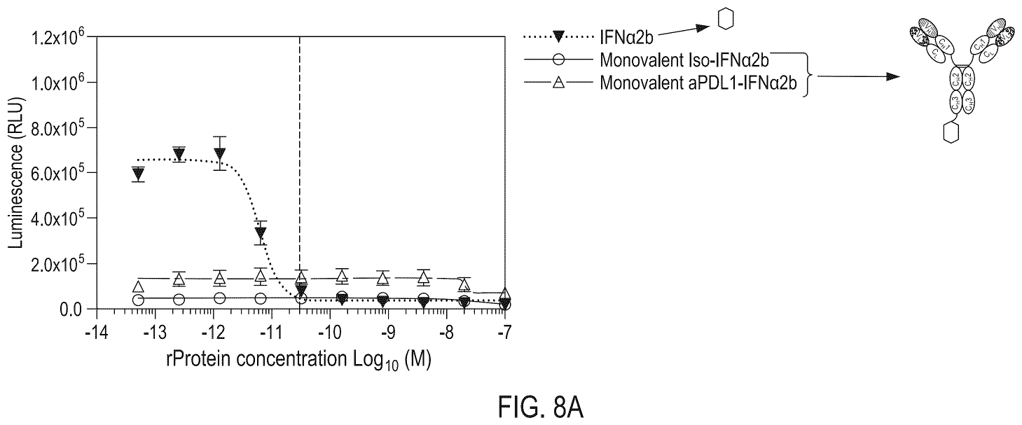

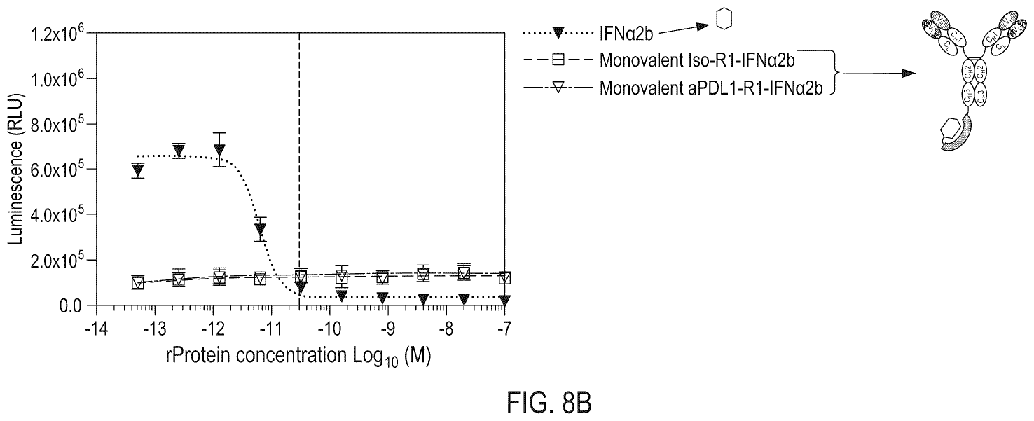

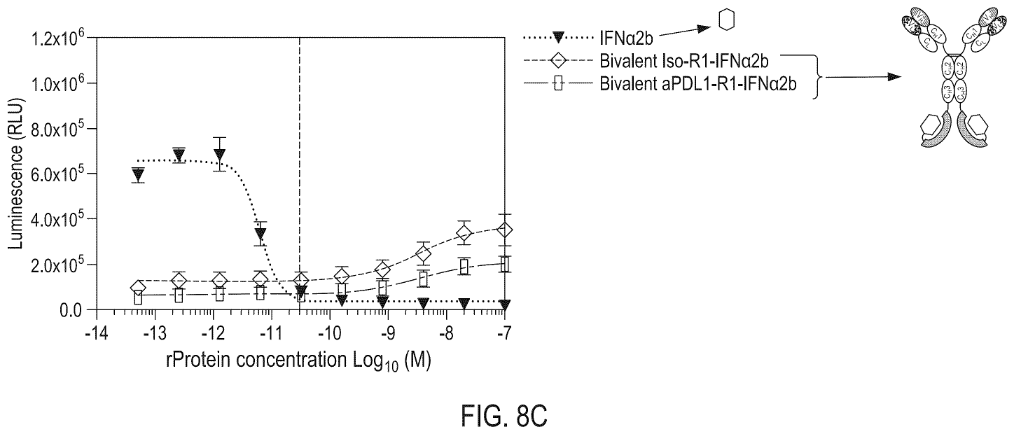

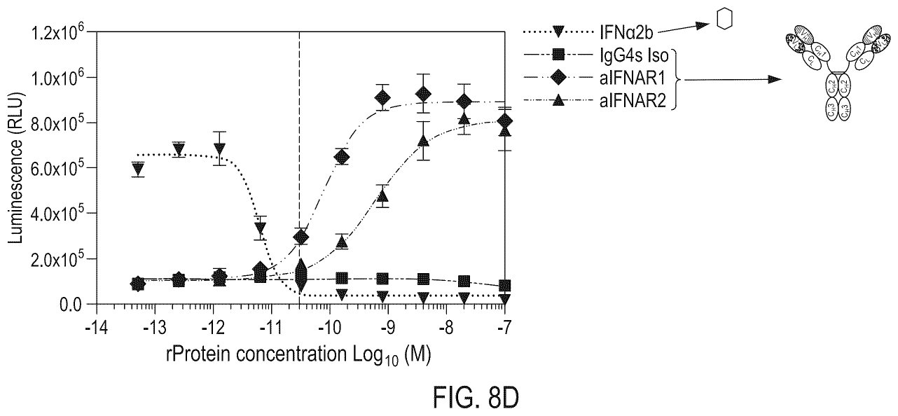

In the second set of assessments, IFNβ was used to trigger cytostatic/cytotoxic response and the cytoprotective activity of the IFN receptor antagonist and control constructs were evaluated in PDL1 OE and PDL1 KO KG-1a/ISRE-Luc reporter cells that were co-treated with 30 pM IFNβ for five days. As observed with the first set of assessments, PDL1 targeted monovalent and bivalent masked IFN receptor antagonist, but not the control constructs, were associated with concentration-dependent increases in luminescence in PDL1 OE KG-1a/ISRE-Luc reporter cells (FIGS. 7A-7C and 8A-8C) and percent inhibition of the IFNα2b treatment-induced cytostatic/cytotoxic response (Table E-2). Again, anti-IFNAR1 and anti-IFNAR2 antibodies yielded relatively modest increases in luminescence which were not PDL1-dependent (FIGS. 70 and 80, and Table E-2). Taken together, these data demonstrate monovalent and bivalent masked IFN receptor antagonists enable potent target-dependent cytoprotective activity.

TABLE E-2
% Inhibition of Cytostaticity/Cytotoxicity

| | PDL1 OE KG-1a/ | | PDL1 KO KG-1a/ | |
|---|---|---|---|---|
| | ISRE-Luc | | ISRE-Luc | |
| | Reporter Cell | | Reporter Cell | |
| Construct | IFNα2b | IFNβ | IFNα2b | IFNβ |
| Unmasked isotype- | 17.7 | 20.4 | 3.2 | 4.8 |
| IFNα2b | | | | |
| Monovalent isotype- | 11.7 | 13.6 | 4.4 | 7.1 |
| R1-IFNα2b | | | | |
| Bivalent isotype-R1- | 17.9 | 18.3 | 33.5 | 43.3 |
| IFNα2b | | | | |
| Unmasked aPDL-1 | 11.5 | 8.1 | 6.4 | 12.2 |
| IFNα2b | | | | |
| Monovalent aPDL1- | 260.7 | 234.8 | 6 | 9.4 |
| R1-IFNα2b | | | | |
| Bivalent aPDL1-R1- | 287.2 | 246.3 | 16.9 | 23.3 |
| IFNα2b | | | | |
| Isotype antibody | 47.9 | 18.9 | 6.6 | 6.7 |
| anti-IFNAR1 | 213.3 | 165.9 | 158.7 | 140.5 |
| (aIFNAR1) | | | | |
| anti-IFNAR2 | 92.1 | 116.4 | 115.1 | 121.8 |
| (aIFNAR2) | | | | |
| IFNα2b | N/A | N/A | N/A | N/A |

### 9.5. Example 4: Blocking Activity of IFN-Induced Cytostaticity by Targeted IFN Receptor Antagonists

Monovalent and bivalent IFNAR1- or IRNAR2-masked constructs were designed and produced as described in Sections 9.1.1 and 9.1.2. Cell viability in the presence of IFNα2b or IFNβ with IFN receptor antagonists and control constructs was evaluated with IFN receptor antagonists and control constructs in PDL1 KO and PDL1 overexpressing (OE) KG-1a/ISRE-Luc reporter cells as described in Section 9.1.6.

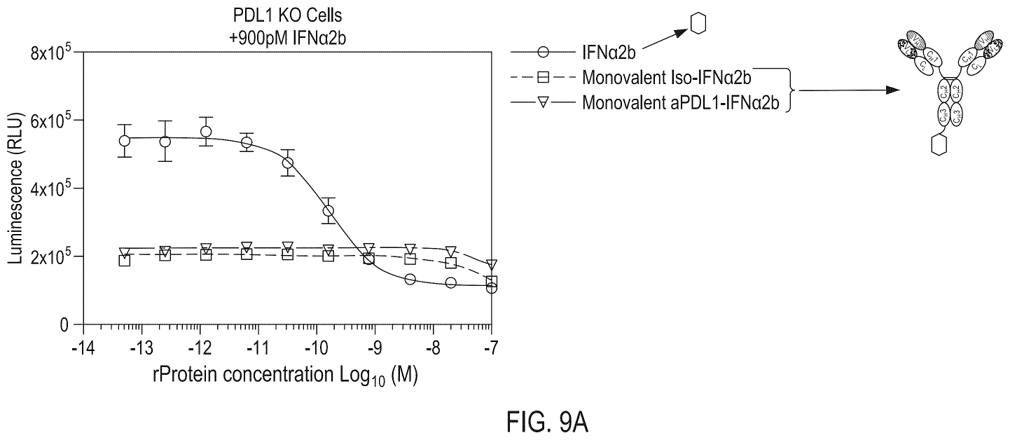

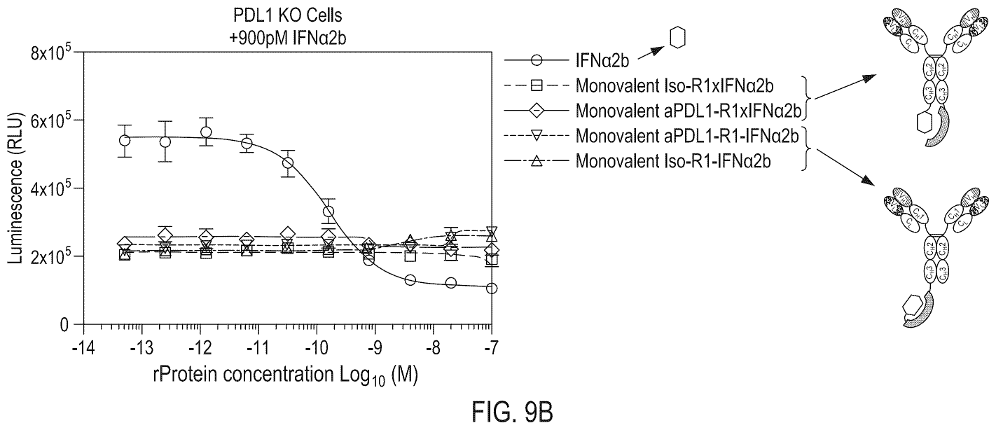

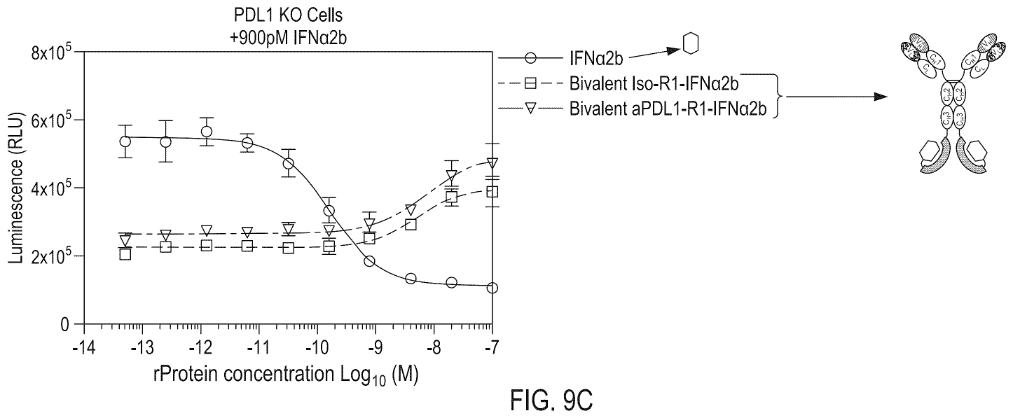

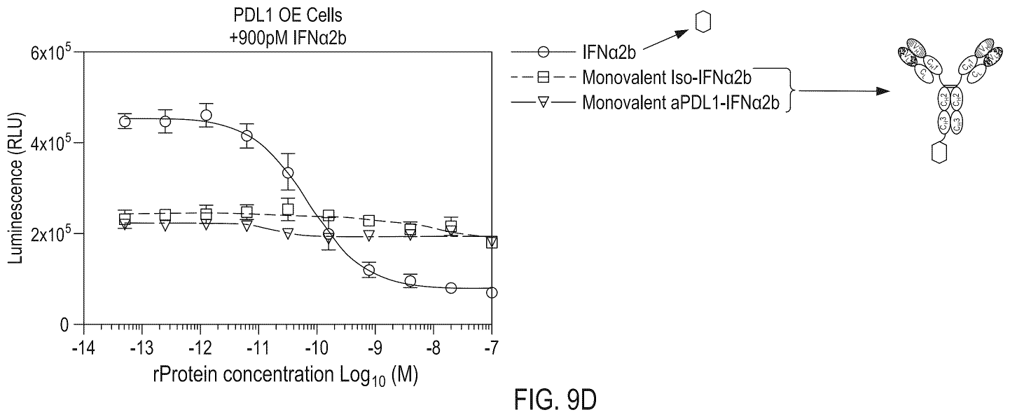

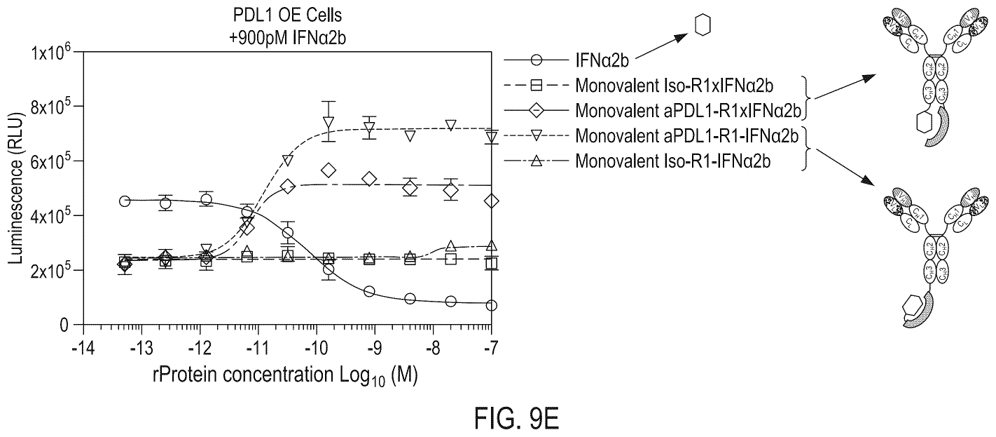

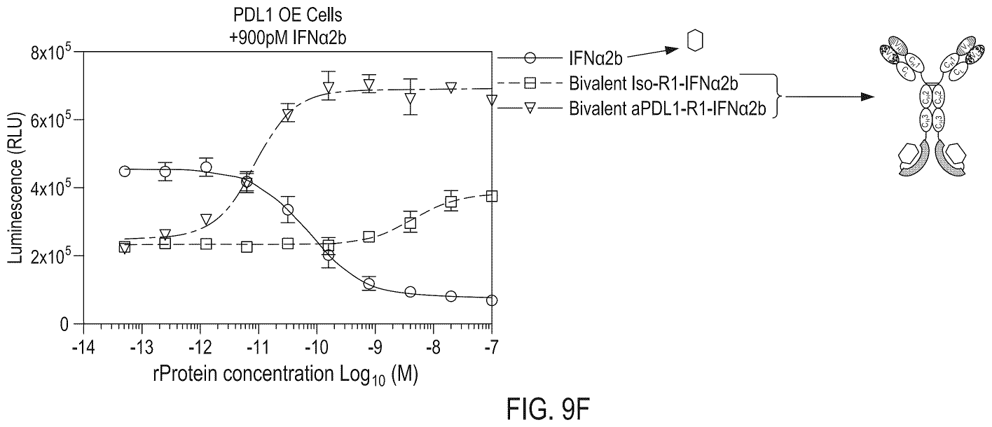

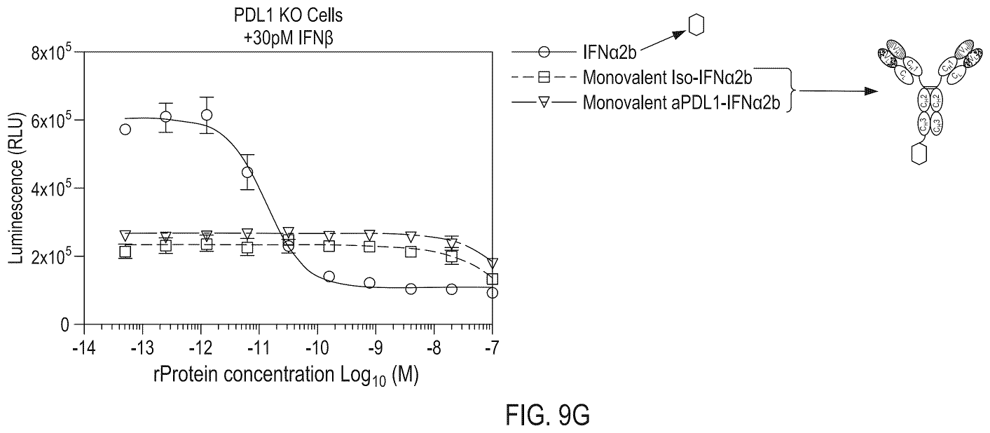

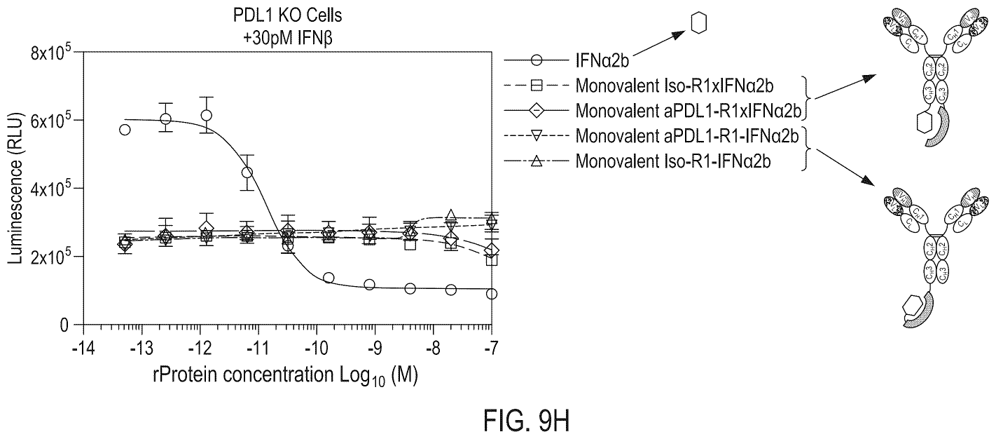

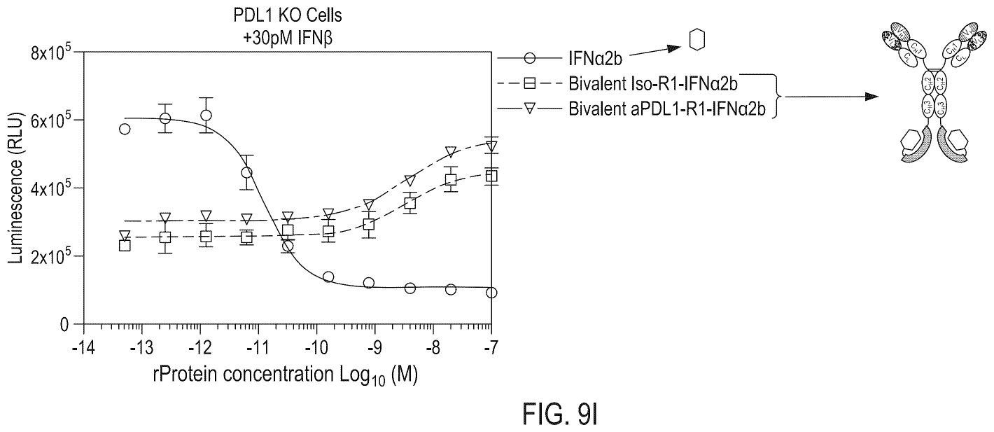

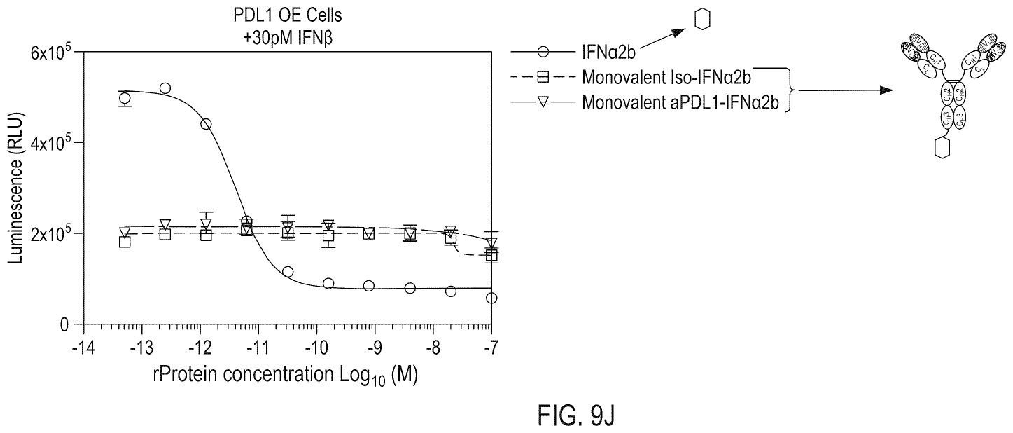

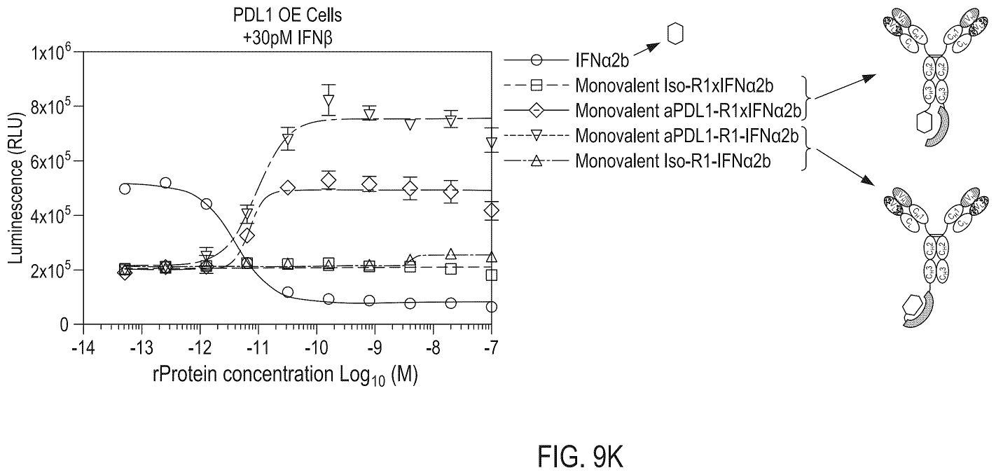

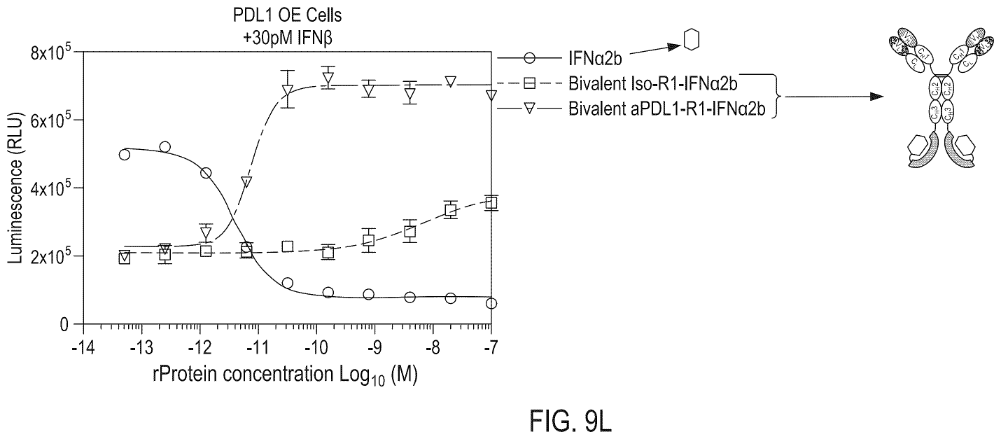

In PDL1-KO cells, IFNα2b had a cytostatic effect in a dose-dependent manner (FIG. 9A). In the presence of IFNα2b, all isotype or PDL1-targeted monovalent IFN receptor antagonists and control constructs showed relatively constant levels of luminescence (FIG. 9B). Targeted and isotype bivalent IFN receptor antagonists were associated with weak blocking of the cytostatic effect in the absence of PD-L1 expression (FIG. 9C).

Next, cell viability was evaluated in PD-L1 OE KG-1a/ISRE-Luc cells in the presence 900 pM IFNα2b with IFN receptor antagonists and control constructs. All control molecules were associated with relatively constant levels of luminescence (FIG. 9D). However, the PD-L1-targeted monovalent and bivalent IFN receptor antagonists were associated with concentration-dependent blocking of the cytostatic effect of IFNα2b (FIGS. 9E and 9F). Both the monovalent and bivalent cis-masked molecules (aPDL1-R1-IFNα2b) achieved significant blocking of IFNα2b cytostatic activity (FIGS. 9E and 9F). The trans-masked molecule (aPDL1-IFNα2b×R1) also achieved significant blocking, although at a lower level compared to the cis-masked monovalent aPDL1-R1-IFNα2b (FIG. 9E).

In the next assessment, cell viability was evaluated in PDL1 KO KG-1a/ISRE-Luc cells in the presence of IFNβ. Again, IFNβ itself also had a cytostatic effect in a dose-dependent manner (FIG. 9G). In the presence of IFNβ, all targeted monovalent IFN receptor antagonists and control constructs showed relatively constant levels of luminescence (FIG. 9H). Targeted and isotype bivalent IFN receptor antagonists were associated with weak blocking of the cytostatic effect (FIG. 91).

Next, cell viability was evaluated in PDL1 OE KG-1a/ISRE-Luc cells in the presence 30 pM IFNβ. All control molecules were associated with relatively constant levels of luminescence (FIGS. 9J-9L). However, the PD-L1-targeted monovalent and bivalent IFN receptor antagonists were associated with concentration-dependent blocking of IFNβ activity. Both the monovalent and bivalent cis-masked molecules (aPDL1-R1-IFNα2b) achieved significant blocking of IFNβ cytostatic activity (FIGS. 9K and 9L). The trans-masked molecule (aPDL1-IFNα2b×R1) also achieved significant blocking, although at a lower level compared to the cis-masked monovalent aPDL1-R1-IFNα2b (FIG. 9K).

### 9.6. Example 5: Effect of Targeted IFN Receptor Antagonists on IP10 Release

Monovalent and bivalent IFNAR1- or IRNAR2-masked constructs were designed and produced as described in Sections 9.1.1 and 9.1.2. The effect of IFNAR1-masked and IFNAR2-masked constructs was assessed in PDL1-expressing monocyte derived DCs (MoDcC), which were incubated with individual constructs for three days, upon which the supernatants were collected and assayed for IP10 with AlphaLISA.

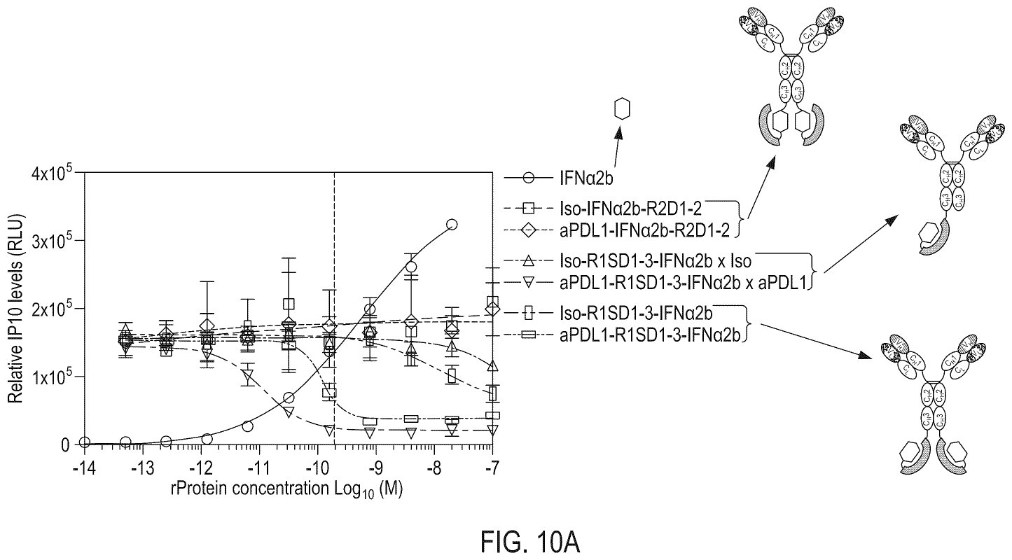

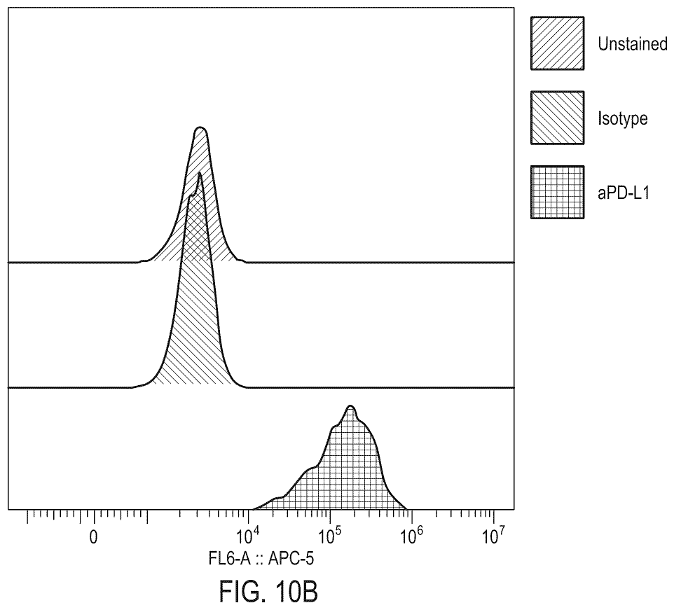

PDL1 expression in MoDCs was confirmed (FIG. 10B). IFNAR1-masked constructs displayed targeting-dependent blocking activity of IFNα2b-mediated IP10 release in MoDCs (FIG. 10A). In contrast, IFNAR2-masked constructs did not block IP10 release (FIG. 10A). Further, monovalent IFNAR1-masked PDL1-targeted construct was associated with higher IP10 release blocking potency compared to the IP10 release blocking potency displayed by the bivalent IFNAR1-masked PDL1-targeted construct (FIG. 10A).

### 9.7. Example 6: In Vitro Signaling Activity of Targeted IFN Receptor Antagonists

Isotype or PDL1-targeted IFN receptor antagonist and control constructs were designed and produced as described in Sections 9.1.1 and 9.1.2. The signaling activity of isotype or PDL1-targeted IFN receptor antagonist and control constructs was assessed using PDL1-KO and PDL1 overexpressing (OE) KG-1a/ISRE-Luc reporter cells as described in Sections 9.1.3 and 9.1.5.

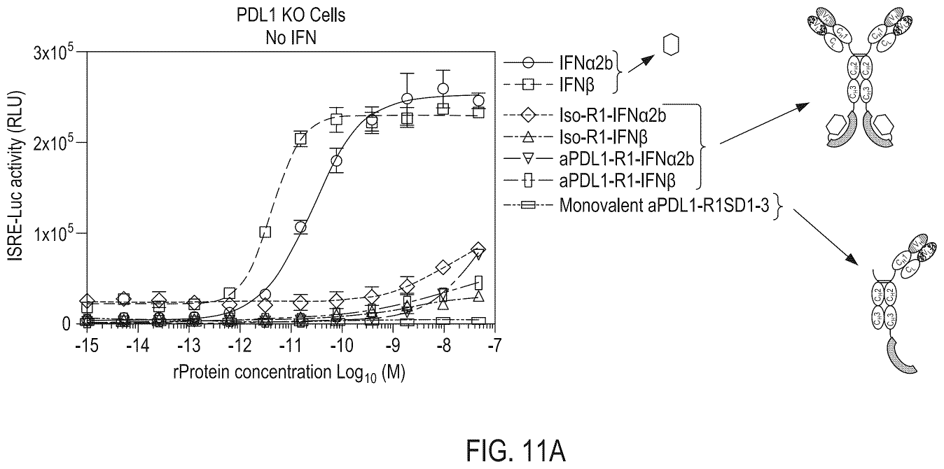

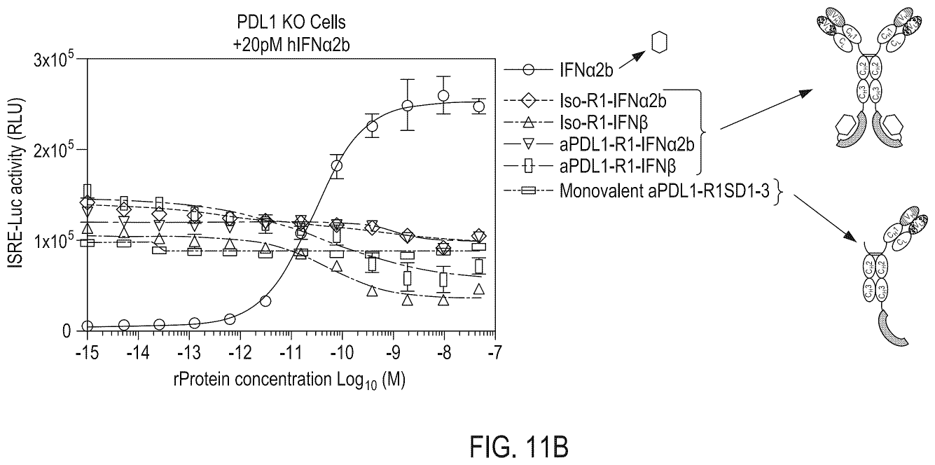

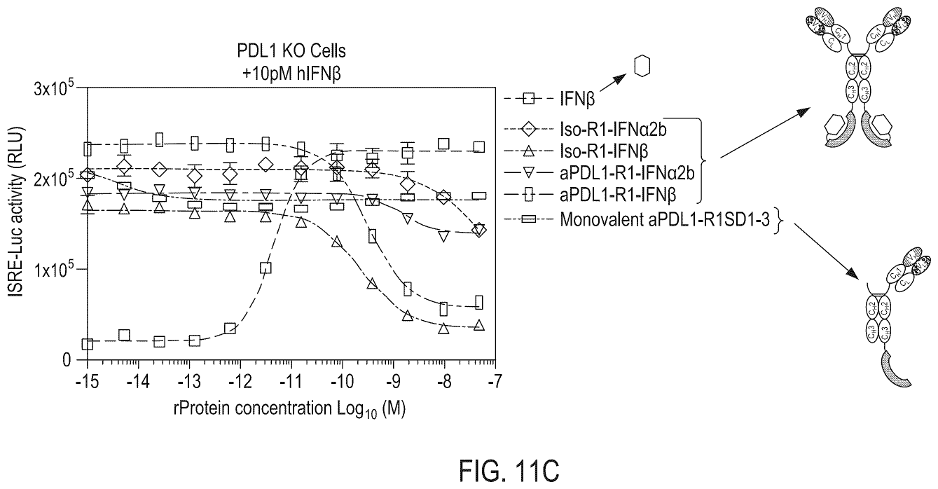

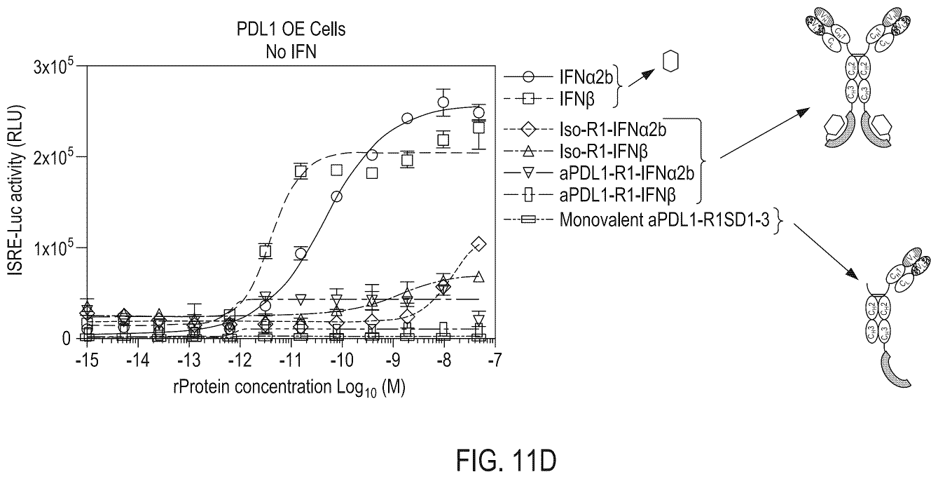

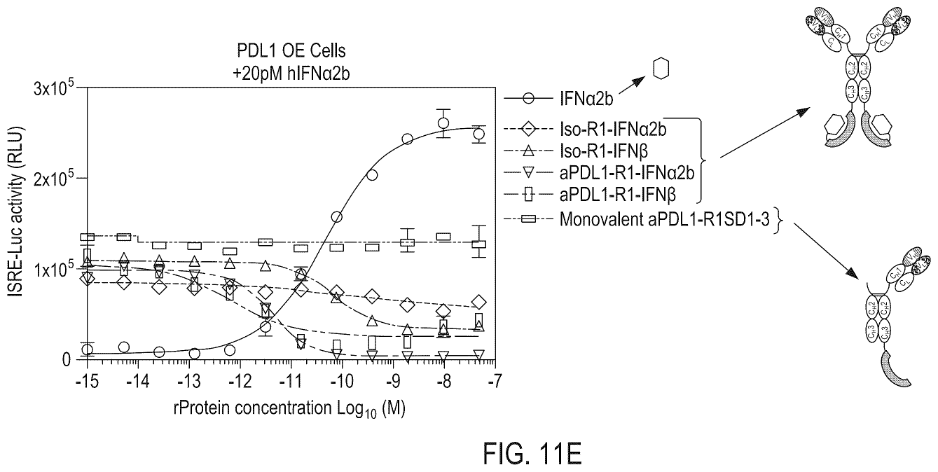

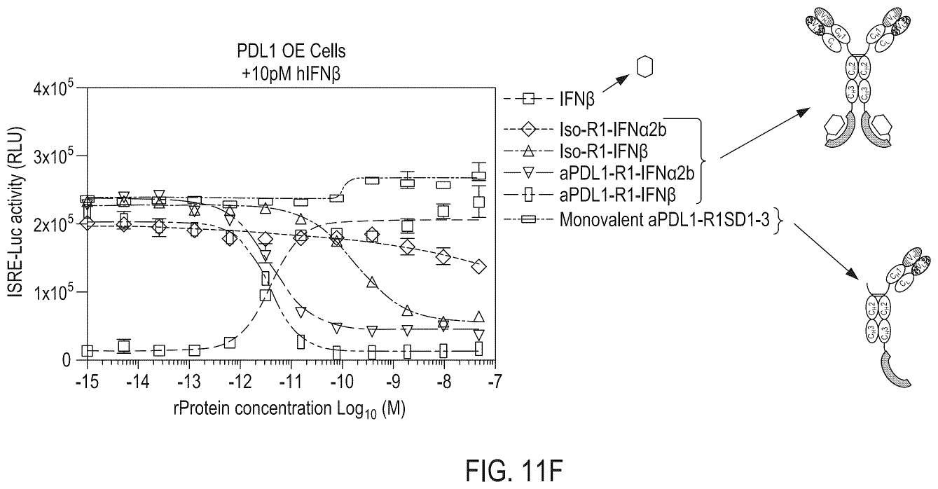

In the first assessment, the activity of isotype or PDL1-targeted bivalent IFN receptor antagonists and control constructs was evaluated in PDL1 KO KG-1a/ISRE-Luc reporter cells. In the absence of hIFNα2b or hIFNβ, all isotype and PDL1-targeted bivalent IFN receptor antagonists displayed relatively low levels of ISRE-Luc activity (FIG. 11A). In the presence of 20 pM hIFNα2b, all bivalent IFN receptor antagonists were associated with a low level of antagonism (FIG. 11B). In the presence of 10 pM hIFNβ, antagonism was observed for constructs containing IFNβ in a targeting-independent manner (FIG. 11C).

Next, the activity of isotype or PDL1-targeted bivalent IFN receptor antagonists and control constructs was evaluated in PDL1 OE KG-1a/ISRE-Luc reporter cells. Again, in the absence of hIFNα2b or hIFNβ, all isotype and PDL1-targeted bivalent IFN receptor antagonists displayed relatively low levels of ISRE-Luc activity (FIG. 11D). In the presence of 20 pM hIFNα2b, both IFNα2b and IFNβ-containing bivalent PDL1-targeted IFN receptor antagonists were associated with significant antagonism (FIG. 11E). In the presence of 10 pM hIFNβ, both IFNα2b and IFNβ-containing bivalent PDL1-targeted IFN receptor antagonists were associated with significant antagonism (FIG. 11F).

In all experiments, aPDL1-R1 constructs lacking any IFN molecule did not show any antagonism (FIGS. 11A-11F).

### 9.1. Example 7: In Vitro Signaling Activity of PDL1- and EGFR-Targeted uIFN-Containing IFN Receptor Antagonists

Isotype, PDL1- or EGFR targeted IFN receptor antagonist and control constructs comprising universal type I interferon (uIFN) moieties were designed and produced as described in Sections 9.1.1 and 9.1.2. The signaling activity of isotype or PDL1-targeted IFN receptor antagonist and control constructs was assessed using PDL1-KO and PDL1-OE KG-1a/ISRE-Luc reporter cells or EGFR OE and no EGFR cells as described in Sections 9.1.3 and 9.1.5.

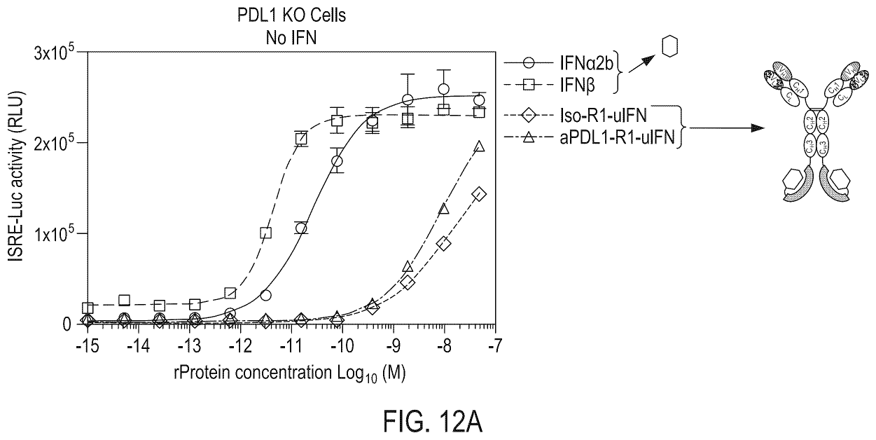

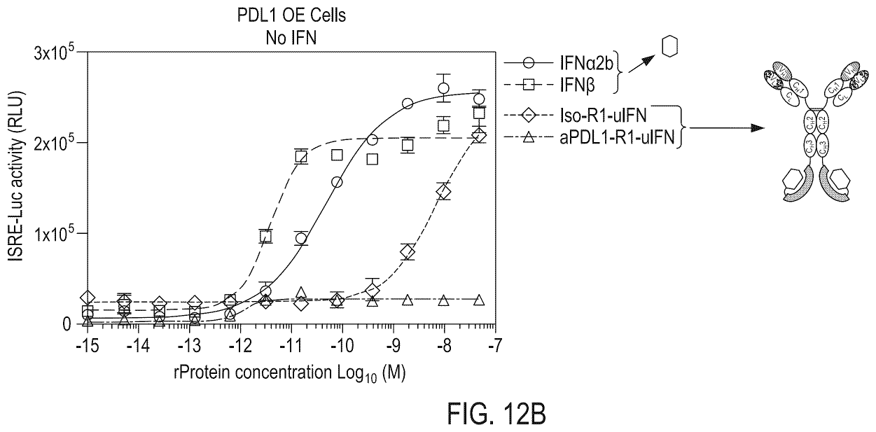

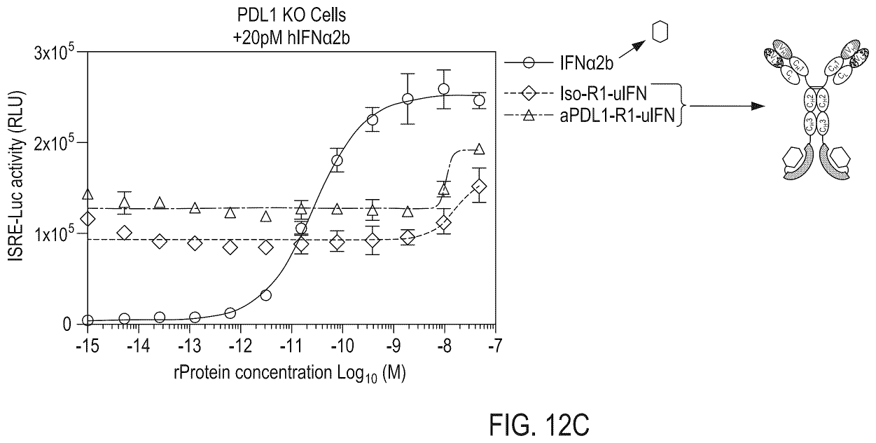

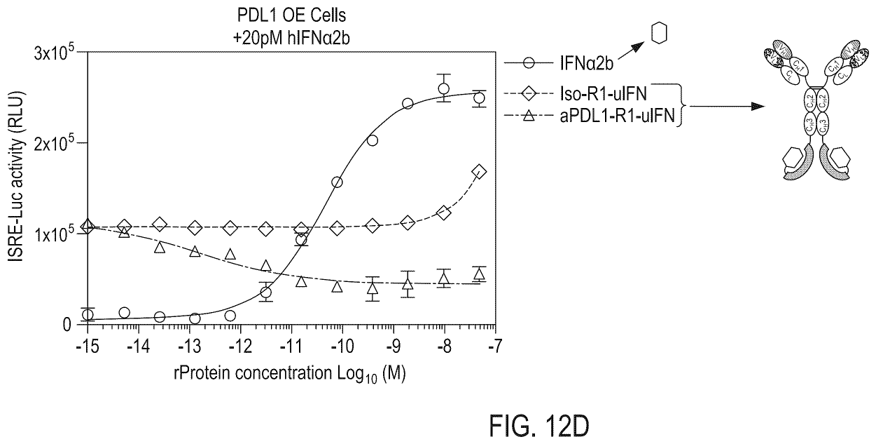

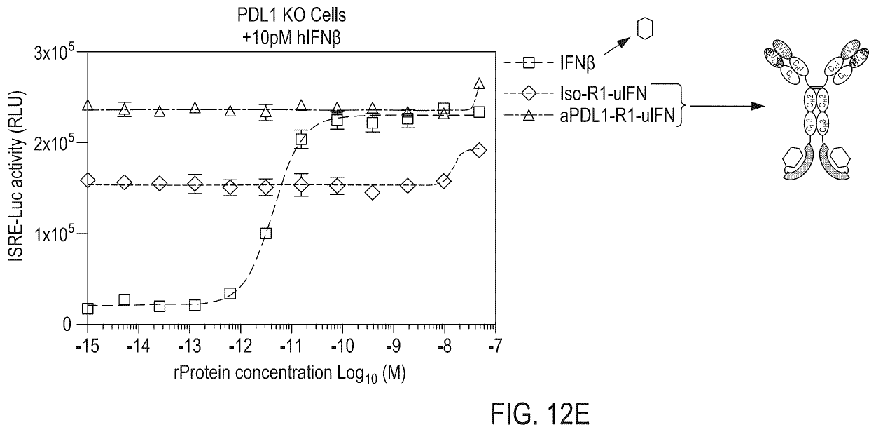

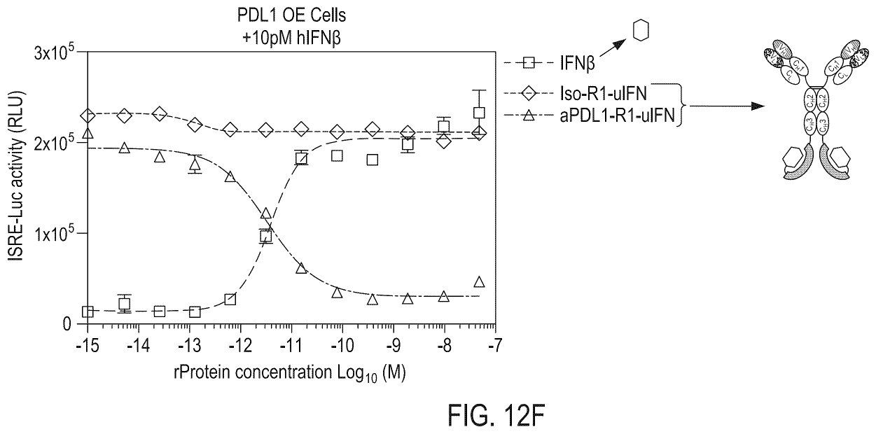

In the absence of hIFNα2b or hIFNβ, both isotype and PDL1-targeted IFN antagonist constructs displayed similar ISRE-Luc activity in PDL1 KO cells (FIG. 12A). Isotype control constructs displayed similar activity in PDL1 OE cells, while PDL1-targeted IFN antagonists showed no activity in these cells (FIG. 12B). In the presence of 20 pM hIFNα2b, both constructs displayed a steady level of ISRE-Luc activity in PDL1 KO cells (FIG. 12C). PDL1-targeted IFN antagonists inhibited this activity in PDL1 OE cells (FIG. 12D), but not isotype controls. Similarly, in the presence of 10 pM hIFNβ, both constructs displayed a steady level of ISRE-Luc activity in PDL1 KO cells (FIG. 12E). Yet, in PDL1 OE cells, the PDL1-targeted IFN antagonist displayed strong antagonism.

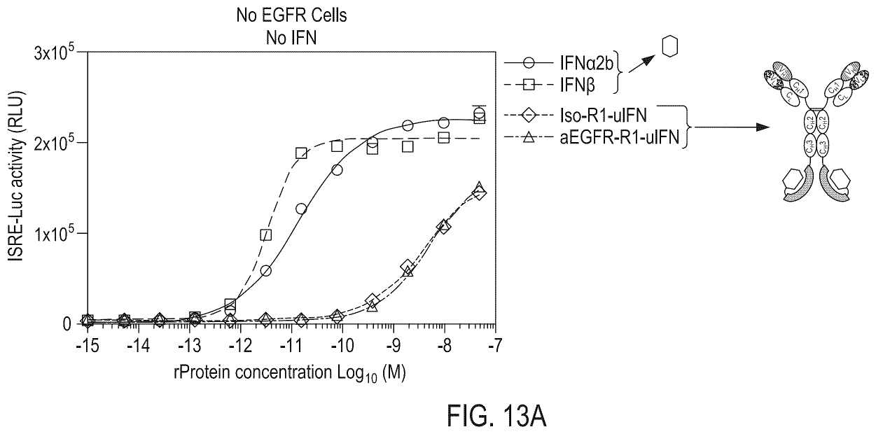

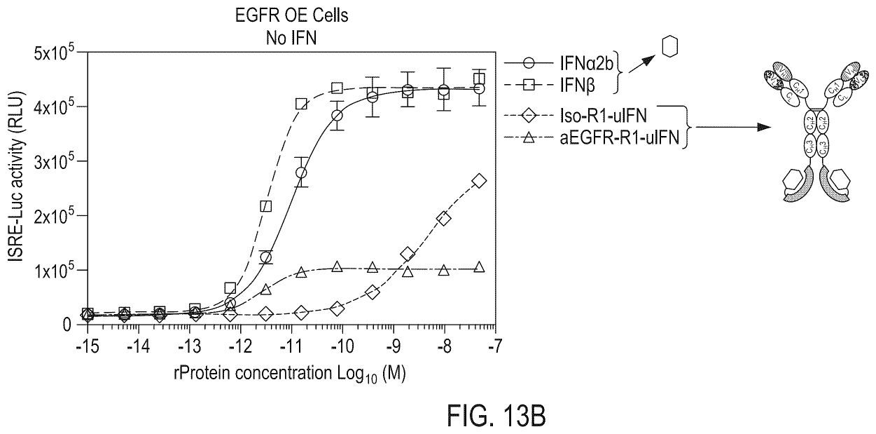

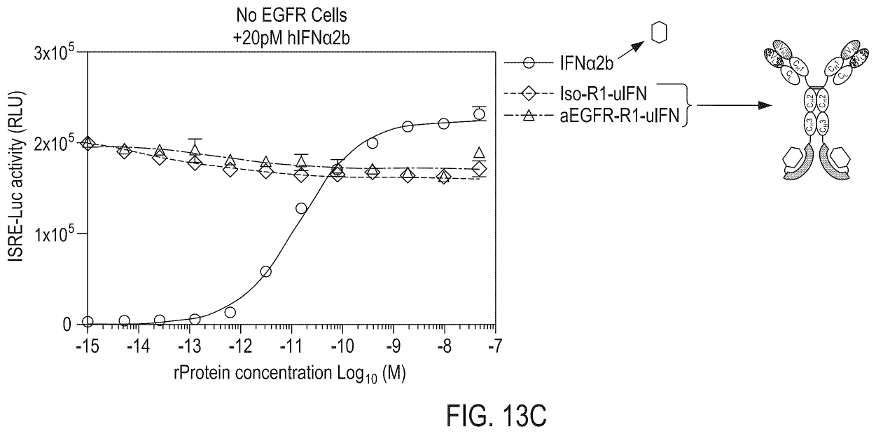

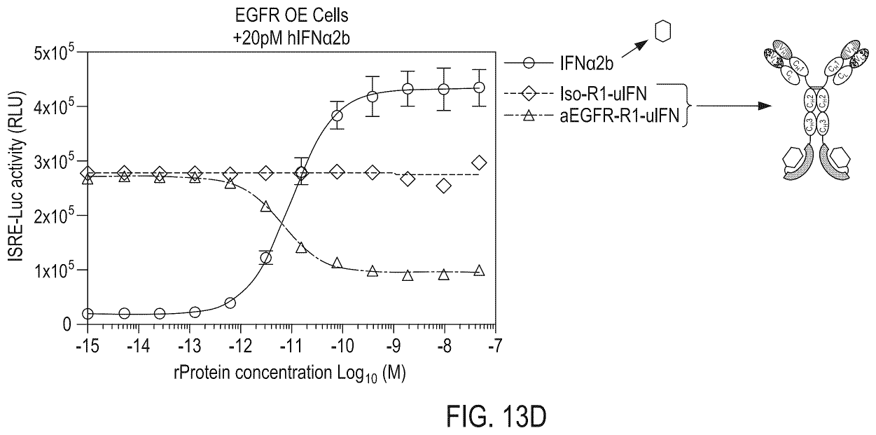

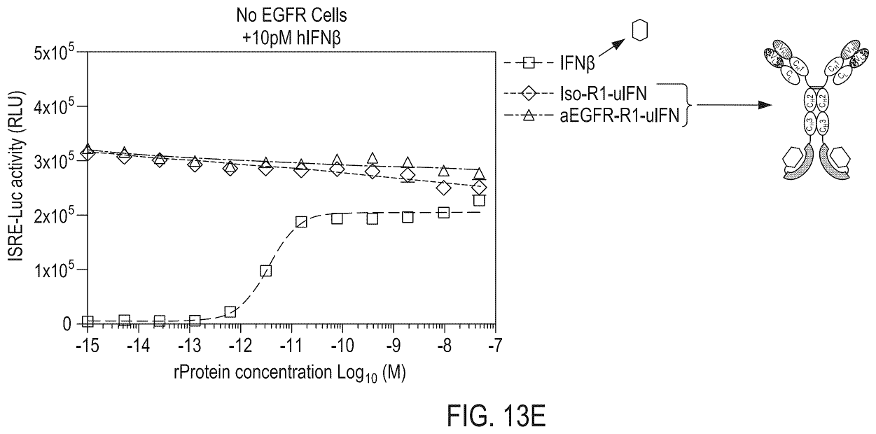

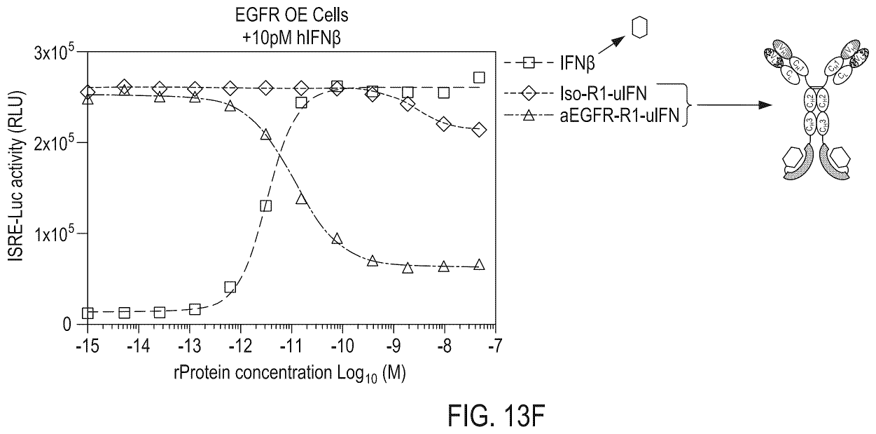

Next, EGFR-targeted and isotype constructs were evaluated. In the absence of hIFNα2b or hIFNβ, both isotype and EGFR-targeted IFN antagonist constructs displayed similar ISRE-Luc activity in no EGFR cells (FIG. 13A). Isotype control constructs displayed similar activity in PDL1 OE cells, while PDL1-targeted IFN antagonists showed reduced activity in these cells (FIG. 13B). In the presence of 20 pM hIFNα2b, both constructs displayed a steady level of ISRE-Luc activity in “no EGFR” cells (FIG. 13C). In EGFR OE cells, EGFR-targeted IFN antagonist displayed significant antagonism (FIG. 13D), but not isotype controls. Similarly, in the presence of 10 pM hIFNβ, both constructs displayed a steady level of ISRE-Luc activity in “no EGFR” cells (FIG. 13E), and again, the EGFR-targeted IFN antagonist displayed significant antagonism in EGFR OE cells.

### 10. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A Type I interferon (IFN) receptor antagonist, comprising:
(a) an anchoring moiety for a cell expressing a Type I interferon receptor;
(b) a Type I interferon alpha receptor 1 (IFNAR1) moiety;
(c) a Type I interferon (IFN) moiety;
(d) a separator moiety connecting the anchoring moiety to the IFNAR1 moiety or the IFN moiety; and
(e) optionally, a linker connecting the IFNAR1 moiety and the IFN moiety.

2. The IFN receptor antagonist of claim 1, wherein the anchoring moiety is capable of binding to an extracellular matrix (ECM) antigen, a tumor reactive lymphocyte antigen, a cell surface molecule of tumor or viral lymphocytes, a T-cell antigen (TCA), a checkpoint inhibitor, a tumor-associated antigen (TAA), a dendritic cell (DC) or other antigen-presenting cell (APC) antigen, or a natural killer (NK) cell antigen.

3. The IFN receptor antagonist of claim 1, wherein the anchoring moiety is a targeting moiety.

4. The IFN receptor antagonist of claim 3, wherein the targeting moiety is an scFv or a Fab.

5. The IFN receptor antagonist of claim 3, wherein the targeting moiety is capable of binding to a checkpoint inhibitor, optionally wherein the checkpoint inhibitor is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.

6. The IFN receptor antagonist of claim 5, wherein the checkpoint inhibitor is PDL1.

7. The IFN receptor antagonist of claim 6, wherein the targeting moiety is an antigen binding fragment of an anti-PDL1 antibody.

8. The IFN receptor antagonist of claim 1, wherein the anchoring moiety comprises means for binding PDL1.

9. The IFN receptor antagonist of any one of claims 1 to 8, wherein the separator moiety is an Fc domain.

10. The IFN receptor antagonist of any one of claims 1 to 9, wherein the linker is a non-cleavable linker (NCL).

11. The IFN receptor antagonist of claim 10, wherein the NCL is a NCL described in Table E.

12. The IFN receptor antagonist of any one of claims 1 to 11, further comprising an additional linker connecting the separator moiety to the IFNAR1 moiety.

13. The IFN receptor antagonist of any one of claims 1 to 11, further comprising an additional linker connecting the separator moiety to the IFN moiety.

14. The IFN receptor antagonist of any one of claims 1 to 13, further comprising an additional anchoring moiety.

15. The IFN receptor antagonist of claim 14, wherein the additional anchoring moiety is an additional targeting moiety.

16. The IFN receptor antagonist of claim 15, wherein the additional targeting moiety is an scFv or a Fab.

17. The IFN receptor antagonist of claim 16, wherein the additional targeting moiety specifically binds to the same target as the anchoring moiety.

18. The IFN receptor antagonist of any one of claims 15 to 17, wherein the additional targeting moiety is capable of binding to a checkpoint inhibitor, optionally CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.

19. The IFN receptor antagonist of claim 18, wherein the checkpoint inhibitor is PDL1.

20. The IFN receptor antagonist of claim 19, wherein the additional targeting moiety is an antigen binding fragment of an anti-PD1 antibody.

21. The IFN receptor antagonist of any one of claims 14 to 20, further comprising an additional separator moiety operably linked to the additional anchoring moiety.

22. The IFN receptor antagonist of any one of claims 1 to 21, wherein the IFN moiety is masked by the IFNAR1 moiety.

23. The IFN receptor antagonist of any one of claims 1 to 22, wherein the IFN receptor antagonist comprises a first polypeptide chain and a second polypeptide chain.

24. The IFN receptor antagonist of claim 23, wherein the first polypeptide chain comprises:
(a) the anchoring moiety or a component thereof;
(b) the separator moiety; and
(c) the IFNAR1 moiety.

25. The IFN receptor antagonist of claim 24, wherein the second polypeptide chain comprises:
(a) an additional anchoring moiety or a component thereof; and
(b) an additional separator moiety.

26. The IFN receptor antagonist of claim 24 or 25, wherein the first polypeptide chain further comprises the IFN moiety.

27. The IFN receptor antagonist of claim 25, wherein the second polypeptide chain further comprises the IFN moiety.

28. The IFN receptor antagonist of claim 27, wherein the second polypeptide chain comprises, in an N- to C-terminal orientation:
(a) an additional anchoring moiety or a component thereof;
(b) an additional separator moiety; and
(c) the IFN moiety.

29. The IFN receptor antagonist of any one of claims 23 to 28, wherein the second polypeptide chain further comprises an additional IFNAR1 moiety and an additional IFN moiety.

30. The IFN receptor antagonist of any one of claims 1 to 29, wherein the IFN moiety comprises an amino acid sequence having at least about 90%, at least about 95%, at least about 98%, or 100% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.

31. The IFN receptor antagonist of claim 30, wherein the IFN moiety comprises an amino acid sequence having at least about 90%, at least about 95%, at least about 98%, or 100% sequence identity to (a) full length mature human IFNα2b or (b) a mature human IFNα2b, having up to a 15-amino acid truncation at its N-terminus and/or its C-terminus.

32. The IFN receptor antagonist of claim 31, wherein the IFN moiety is (a) full length mature human IFNα2b or (b) a mature human IFNα2b, having up to a 15-amino acid truncation at its N-terminus and/or its C-terminus.

33. The IFN receptor antagonist of any one of claims 1 to 32, wherein the IFNAR1 moiety comprises or consists of an amino acid sequence having at least 90%, at least 95%, at least 98%, or 100% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.

34. The IFN receptor antagonist of claim 29, wherein the additional IFN moiety comprises an amino acid sequence having at least about 90%, at least about 95%, at least about 98%, or 100% sequence identity to (a) full length mature human IFNα1, IFNα2b, IFNβ, IFNω, IFNε or IFNκ or (b) a mature human IFNα1, IFNα2b, IFNβ, IFNω, IFNε or IFNκ having up to a 15-amino acid truncation at its N-terminus and/or its C-terminus.

35. The IFN receptor antagonist of claim 34, wherein the additional IFN moiety comprises an amino acid sequence having at least about 90%, at least about 95%, at least about 98%, or 100% sequence identity to (a) full length mature human IFNα2b or (b) a mature human IFNα2b, having up to a 15-amino acid truncation at its N-terminus and/or its C-terminus.

36. The IFN receptor antagonist of claim 29, wherein the additional IFNAR1 moiety comprises or consists of an amino acid sequence having at least 90%, at least 95%, or at least 98% sequence identity to (i) the SD2 and SD3 domains of human IFNAR1, (ii) the SD1, SD2 and SD3 domains of human IFNAR1, or (iii) the SD1, SD2, SD3 and SD4 domains of human IFNAR1.

37. The IFN receptor antagonist of claim 29, wherein the additional IFNAR1 moiety is (i) the SD2 and SD3 domains of human IFNAR1, (ii) the SD1, SD2 and SD3 domains of human IFNAR1, or (iii) the SD1, SD2, SD3 and SD4 domains of human IFNAR1.

38. The IFN receptor antagonist of any one of claims 1 to 37, wherein the IFN receptor antagonist does not comprise an IFNAR2 moiety.

39. The IFN receptor antagonist of claim 38, wherein the IFNAR2 moiety comprises or consists of an amino acid sequence having at least 90%, at least 95%, or at least 98% sequence identity to (i) the D1 domain of human IFNAR2 or (ii) the D1 and D2 domains of human IFNAR2.

40. An IFN receptor antagonist, which is optionally an IFN receptor antagonist according to any one of claims 1 to 39, which comprises polypeptide chains having (a) the configuration of the two half-antibodies illustrated in FIG. 1B, (b) the configuration of the two half-antibodies illustrated in FIG. 1C, (c) the configuration of the two half-antibodies illustrated in FIG. 1D, (d) the configuration of the two half-antibodies illustrated in FIG. 1E, (e) the configuration of the two half-antibodies illustrated in FIG. 1F.

41. A nucleic acid or plurality of nucleic acids encoding the IFN receptor antagonist of any one of claims 1 to 40.

42. A host cell engineered to express the IFN receptor antagonist of any one of claims 1 to 40 or the nucleic acid(s) of claim 41.

43. A method of producing the IFN receptor antagonist of any one of claims 1 to 40, comprising culturing the host cell of claim 42 and recovering the IFN receptor antagonist expressed thereby.

44. A pharmaceutical composition comprising the IFN receptor antagonist of any one of claims 1 to 40 and an excipient, optionally further comprising an oncolytic virus.

45. A method of inhibiting IFN signaling in a cell expressing (a) an IFN receptor and (b) a molecule to which the anchoring moiety specifically binds, the method comprising contacting the cell with the IFN receptor antagonist of any one of claims 1 to 40 or the pharmaceutical composition of claim 44.

46. The method of claim 45, wherein the cell is (a) an immune cell, optionally a B cell, or (b) a cancer cell.

47. The method of claim 45 or 46, wherein the method is an in vitro method.

48. A method of treating a subject suffering from cancer, the method comprising administering to a subject in need thereof the IFN receptor antagonist of any one of claims 1 to 40 or the pharmaceutical composition of claim 44.

49. The method of claim 48, further comprising administering to the subject an oncolytic virus.

50. A method of treating a subject suffering from an immune disorder or condition comprising administering to the subject the IFN receptor antagonist of any one of claims 1 to 40 or the pharmaceutical composition of claim 44, optionally wherein the immune disorder or condition is systemic lupus erythematosus (SLE), rheumatoid arthritis (RA), systemic sclerosis (SSc), or Sjogren's syndrome (SS).


SEQUENCE LISTING (151 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 166 aa, synthetic construct
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 4 - protein, 166 aa, synthetic construct
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNVDSILAVKKYFRRITLYLTEKKYSPCAWEVVRAEIMRSLSLSTNLQERLRRKE

SEQ ID NO: 5 - protein, 165 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 6 - protein, 165 aa, synthetic construct [sequence omitted]

SEQ ID NO: 7 - protein, 165 aa, synthetic construct
CDLPQTHSLGSRRTLMLLAQMRRISLFSCLKDAHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 8 - protein, 165 aa, synthetic construct
CDLPQTHSLGSRRTLMLLAQMRRISLFSCLKDKHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 9 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYAQLNDLEACVIQGVGVTETPLMKEDSILAVRKYFQRITLYLKEKKYSPCAWEVVRAEIMRSFSLSTNLQESLRSKE

SEQ ID NO: 10 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLAACVIQGVGVTETPLMKEDSILAVRKYFQRITLYLKEKKYSPCAWEVVRAEIMRSFSLSTNLQESLRSKE

SEQ ID NO: 11 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVAKYFQRITLYLKEKKYSPCAWEVVRAEIMRSFSLSTNLQESLRSKE

SEQ ID NO: 12 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRMEIMRSFSLSTNLQESLRSKE

SEQ ID NO: 13 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 14 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMKSFSLSTNLQESLRSKE

SEQ ID NO: 15 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFALSTNLQESLRSKE

SEQ ID NO: 16 - protein, 153 aa, synthetic construct
THSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQ

SEQ ID NO: 17 - protein, 158 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQ

SEQ ID NO: 18 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 19 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 20 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 21 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDKNENEDMKEMKENEMKPSEARVPQLSSLELRRYFHRIDNFLKEKKYSDCAWEIVRVEIRRCLYYFYKFTALFRRK

SEQ ID NO: 22 - protein, 557 aa, Homo sapiens
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 23 - protein, 35 aa, synthetic construct
GGGGSLALGPGGGGGSLALGPGGGGGSLALGPGGS

SEQ ID NO: 24 - protein, 25 aa, synthetic construct
GGGGSGGGGSGGGGSGGGGSGGGGS

SEQ ID NO: 25 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 26 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 27 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 28 - protein, 20 aa, synthetic construct
  VARIANT 1..20: note = This sequence may encompass 1-10 GS repeating units
GSGSGSGSGSGSGSGSGSGS

SEQ ID NO: 29 - protein, 50 aa, synthetic construct
  VARIANT 1..50: note = This sequence may encompass 1-10 GSGGS repeating units
GSGGSGSGGSGSGGSGSGGSGSGGSGSGGSGSGGSGSGGSGSGGSGSGGS

SEQ ID NO: 30 - protein, 5 aa, synthetic construct
ADAAP

SEQ ID NO: 31 - protein, 11 aa, synthetic construct
ADAAPTVSIFP

SEQ ID NO: 32 - protein, 12 aa, synthetic construct
ADAAPTVSIFPP

SEQ ID NO: 33 - protein, 6 aa, synthetic construct
AKTTAP

SEQ ID NO: 34 - protein, 13 aa, synthetic construct
AKTTAPSVYPLAP

SEQ ID NO: 35 - protein, 17 aa, synthetic construct
AKTTPKLEEGEFSEARV

SEQ ID NO: 36 - protein, 9 aa, synthetic construct
AKTTPKLGG

SEQ ID NO: 37 - protein, 6 aa, synthetic construct
AKTTPP

SEQ ID NO: 38 - protein, 13 aa, synthetic construct
AKTTPPSVTPLAP

SEQ ID NO: 39 - protein, 6 aa, synthetic construct
ASTKGP

SEQ ID NO: 40 - protein, 26 aa, synthetic construct
ASTKGPSVFPLAPASTKGPSVFPLAP

SEQ ID NO: 41 - protein, 14 aa, synthetic construct
EGKSSGSGSESKST

SEQ ID NO: 42 - protein, 15 aa, synthetic construct
GEGESGEGESGEGES

SEQ ID NO: 43 - protein, 20 aa, synthetic construct
GEGESGEGESGEGESGEGES

SEQ ID NO: 44 - protein, 15 aa, synthetic construct
GEGGSGEGGSGEGGS

SEQ ID NO: 45 - protein, 15 aa, synthetic construct
GENKVEYAPALMALS

SEQ ID NO: 46 - protein, 15 aa, synthetic construct
GGEGSGGEGSGGEGS

SEQ ID NO: 47 - protein, 15 aa, synthetic construct
GGGESGGEGSGEGGS

SEQ ID NO: 48 - protein, 15 aa, synthetic construct
GGGESGGGESGGGES

SEQ ID NO: 49 - protein, 10 aa, synthetic construct
GGGGSGGGGS

SEQ ID NO: 50 - protein, 15 aa, synthetic construct
GGGGSGGGGSGGGGS

SEQ ID NO: 51 - protein, 20 aa, synthetic construct
GGGGSGGGGSGGGGSGGGGS

SEQ ID NO: 52 - protein, 15 aa, synthetic construct
GGGKSGGGKSGGGKS

SEQ ID NO: 53 - protein, 15 aa, synthetic construct
GGGKSGGKGSGKGGS

SEQ ID NO: 54 - protein, 4 aa, synthetic construct
GGGS

SEQ ID NO: 55 - protein, 5 aa, synthetic construct
GGGSG

SEQ ID NO: 56 - protein, 15 aa, synthetic construct
GGKGSGGKGSGGKGS

SEQ ID NO: 57 - protein, 4 aa, synthetic construct
GGSG

SEQ ID NO: 58 - protein, 5 aa, synthetic construct
GGSGG

SEQ ID NO: 59 - protein, 9 aa, synthetic construct
GGSGGGGSG

SEQ ID NO: 60 - protein, 13 aa, synthetic construct
GGSGGGGSGGGGS

SEQ ID NO: 61 - protein, 15 aa, synthetic construct
GHEAAAVMQVQYPAS

SEQ ID NO: 62 - protein, 15 aa, synthetic construct
GKGGSGKGGSGKGGS

SEQ ID NO: 63 - protein, 15 aa, synthetic construct
GKGKSGKGKSGKGKS

SEQ ID NO: 64 - protein, 20 aa, synthetic construct
GKGKSGKGKSGKGKSGKGKS

SEQ ID NO: 65 - protein, 15 aa, synthetic construct
GKPGSGKPGSGKPGS

SEQ ID NO: 66 - protein, 21 aa, synthetic construct
GKPGSGKPGSGKPGSGKPSGS

SEQ ID NO: 67 - protein, 15 aa, synthetic construct
GPAKELTPLKEAKVS

SEQ ID NO: 68 - protein, 12 aa, synthetic construct
GSAGSAAGSGEF

SEQ ID NO: 69 - protein, 5 aa, synthetic construct
GSGGG

SEQ ID NO: 70 - protein, 5 aa, synthetic construct
GSGSG

SEQ ID NO: 71 - skipped

SEQ ID NO: 72 - protein, 4 aa, synthetic construct
GSSG

SEQ ID NO: 73 - protein, 10 aa, synthetic construct
GSSGGSGGSG

SEQ ID NO: 74 - protein, 11 aa, synthetic construct
GSSGGSGGSGG

SEQ ID NO: 75 - protein, 12 aa, synthetic construct
GSSGGSGGSGGS

SEQ ID NO: 76 - protein, 13 aa, synthetic construct
GSSGGSGGSGGSG

SEQ ID NO: 77 - protein, 16 aa, synthetic construct
GSSGGSGGSGGSGGGS

SEQ ID NO: 78 - protein, 11 aa, synthetic construct
GSSGGSGGSGS

SEQ ID NO: 79 - protein, 5 aa, synthetic construct
GSSGT

SEQ ID NO: 80 - protein, 5 aa, synthetic construct
GSSSG

SEQ ID NO: 81 - protein, 18 aa, synthetic construct
GSTSGSGKPGSGEGSTKG

SEQ ID NO: 82 - protein, 19 aa, synthetic construct
GTAAAGAGAAGGAAAGAAG

SEQ ID NO: 83 - protein, 19 aa, synthetic construct
GTSGSSGSGSGGSGSGGGG

SEQ ID NO: 84 - protein, 14 aa, synthetic construct
IRPRAIGGSKPRVA

SEQ ID NO: 85 - protein, 18 aa, synthetic construct
KESGSVSSEQLAQFRSLD

SEQ ID NO: 86 - protein, 15 aa, synthetic construct
KTTPKLEEGEFSEAR

SEQ ID NO: 87 - protein, 19 aa, synthetic construct
PRGASKSGSASQTGSAPGS

SEQ ID NO: 88 - protein, 6 aa, synthetic construct
QPKAAP

SEQ ID NO: 89 - protein, 13 aa, synthetic construct
QPKAAPSVTLFPP

SEQ ID NO: 90 - protein, 27 aa, synthetic construct
RADAAAAGGGGSGGGGSGGGGSGGGGS

SEQ ID NO: 91 - protein, 12 aa, synthetic construct
RADAAAAGGPGS

SEQ ID NO: 92 - protein, 6 aa, synthetic construct
RADAAP

SEQ ID NO: 93 - protein, 9 aa, synthetic construct
RADAAPTVS

SEQ ID NO: 94 - protein, 6 aa, synthetic construct
SAKTTP

SEQ ID NO: 95 - protein, 18 aa, synthetic construct
SAKTTPKLEEGEFSEARV

SEQ ID NO: 96 - protein, 10 aa, synthetic construct
SAKTTPKLGG

SEQ ID NO: 97 - protein, 14 aa, synthetic construct
STAGDTHLGGEDFD

SEQ ID NO: 98 - protein, 5 aa, synthetic construct
TVAAP

SEQ ID NO: 99 - protein, 12 aa, synthetic construct
TVAAPSVFIFPP

SEQ ID NO: 100 - protein, 24 aa, synthetic construct
TVAAPSVFIFPPTVAAPSVFIFPP

SEQ ID NO: 101 - protein, 19 aa, synthetic construct
YNLLGFLQRSSNFQCQKLL

SEQ ID NO: 102 - protein, 19 aa, synthetic construct
KEDAALTIYEMLQNIFAIF

SEQ ID NO: 103 - protein, 26 aa, synthetic construct
ETIVENLLANVYHQINHLKTVLEEKL

SEQ ID NO: 104 - protein, 18 aa, synthetic construct
SSLHLKRYYGRILHYLKA

SEQ ID NO: 105 - protein, 22 aa, synthetic construct
HCAWTIVRVEILRNFYFINRLT

SEQ ID NO: 106 - protein, 11 aa, synthetic construct
  VARIANT 1..10: note = This region may encompass 1-10 residues
GGGGGGGGGGS

SEQ ID NO: 107 - protein, 11 aa, synthetic construct
  VARIANT 2..11: note = This region may encompass 1-10 residues
SGGGGGGGGGG

SEQ ID NO: 108 - protein, 5 aa, synthetic construct
GGGGS

SEQ ID NO: 109 - protein, 4 aa, synthetic construct
GGGG

SEQ ID NO: 110 - protein, 5 aa, synthetic construct
GGGGG

SEQ ID NO: 111 - protein, 6 aa, synthetic construct
GGGGGG

SEQ ID NO: 112 - protein, 7 aa, synthetic construct
GGGGGGG

SEQ ID NO: 113 - protein, 8 aa, synthetic construct
GGGGGGGG

SEQ ID NO: 114 - protein, 9 aa, synthetic construct
GGGGGGGGG

SEQ ID NO: 115 - protein, 231 aa, synthetic construct
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 120 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 121 - protein, 4 aa, synthetic construct
CPPC

SEQ ID NO: 122 - protein, 4 aa, Homo sapiens
CPSC

SEQ ID NO: 123 - protein, 20 aa, synthetic construct
EPKSCDKTHTCPPCPAPPVA

SEQ ID NO: 124 - protein, 17 aa, synthetic construct
ESKYGPPCPPCPAPPVA

SEQ ID NO: 125 - protein, 15 aa, synthetic construct
CPPCPAPGGGGPSVF

SEQ ID NO: 126 - protein, 14 aa, synthetic construct
CPPCPAPGGGPSVF

SEQ ID NO: 127 - protein, 13 aa, synthetic construct
CPPCPAPGGPSVF

SEQ ID NO: 128 - protein, 12 aa, synthetic construct
CPPCPAPGPSVF

SEQ ID NO: 129 - protein, 408 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 130 - protein, 715 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 131 - protein, 408 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 132 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 133 - protein, 545 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGKGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQ

SEQ ID NO: 134 - protein, 715 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 135 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 545 aa, synthetic construct; identical to SEQ ID NO: 133

SEQ ID NO: 137 - protein, 165 aa, synthetic construct
CDLPQTHSLGSRRTLMLLAQMRKISLFSCLKDRHDFGFPQEEFGNQFQKAETIPVLHEMIQQIFNLFTTKDSSAAWDEDLLDKFCTELYQQLNDLEACVMQEERVGETPLMNVDSILAVKKYFRRITLYLTEKKYSPCAWEVVRAEIMRSLSLSTNLQERLRRKE

SEQ ID NO: 138 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVEKYFQRITLYLKEKKYSPCAWEVVRAEIMRSFSLSTNLQESLRSKE

SEQ ID NO: 139 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVEKYFQRITLYLKEKKYSPCAWEVVRAEIMRSFSLSTNLQKRLKSKE

SEQ ID NO: 140 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 142 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 143 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 145 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 148 - protein, 235 aa, synthetic construct; identical to SEQ ID NO: 116

SEQ ID NO: 149 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 151 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK